SEQUENCE LISTING (19 sequences)

SEQ ID NO: 1 - DNA, 3822 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 194 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 4 - DNA, 3768 bp, Severe acute respiratory syndrome coronavirus
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 5 - protein, 1255 aa, Severe acute respiratory syndrome coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 6 - protein, 193 aa, Severe acute respiratory syndrome coronavirus
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATV

SEQ ID NO: 7 - DNA, 4059 bp, Middle East respiratory syndrome coronavirus
atgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcac

SEQ ID NO: 8 - protein, 1353 aa, Middle East respiratory syndrome coronavirus
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 9 - protein, 212 aa, Middle East respiratory syndrome coronavirus
QAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 10 - protein, 605 aa, Aspergillus niger
MQTLLVSSLVVSLAAALPHYIRSNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 11 - DNA, 68 bp, Artificial Sequence
  synthetic oligonucleotide
  misc_feature (1)..(1): 5′ biotin
aattaaccctcactaaagggctgagtctcaaaaccgcaatacactggttgtatggtcgaataagttaa

SEQ ID NO: 12 - DNA, 51 bp, Artificial Sequence
  synthetic oligonucleotide
  misc_feature (1)..(1): 5′ biotin
cagcaccgaccttgtgctttgggagtgctggtccaagggcgttaatggaca

SEQ ID NO: 13 - DNA, 76 bp, Artificial Sequence
  synthetic oligonucleotide
  misc_feature (1)..(1): 5′ biotin
atccagagtgacgcagcaccgaccttgtgctttgggagtgctggtccaagggcgttaatggacacggtggcttagt

SEQ ID NO: 14 - DNA, 76 bp, Artificial Sequence
  synthetic oligonucleotide
  misc_feature (1)..(1): 5′ biotin
atccagagtgacgcagcatcgagtggtgggctggtcgggtttggattcccttagatgctggacacggtggcttagt

SEQ ID NO: 15 - DNA, 77 bp, Artificial Sequence
  synthetic oligonucleotide
  misc_feature (1)..(1): 5′ biotin
atccagagtgacgcagcactgcgtaggcgcggccaatgtgtaggattgctcaggtctgctggacacggtggcttagt

SEQ ID NO: 16 - DNA, 76 bp, Artificial Sequence
  synthetic oligonucleotide
  misc_feature (1)..(1): 5′ biotin
atccagagtgacgcagcatttcatcgggtccaaaaggggctgctcgggattgcggatatggacacggtggcttagt

SEQ ID NO: 17 - DNA, 76 bp, Artificial Sequence
  synthetic oligonucleotide
  misc_feature (1)..(1): 5′ biotin
atccagagtgacgcagcaggactgcttaggattgcgaagctgaggagctcccccgccttggacacggtggcttagt

SEQ ID NO: 18 - DNA, 76 bp, Artificial Sequence
  synthetic oligonucleotide
  misc_feature (1)..(1): 5′ biotin
atccagagtgacgcagcagtagggggattggctccagggcctggctgacggttgcacgtggacacggtggcttagt

SEQ ID NO: 19 - DNA, 67 bp, Artificial Sequence
  synthetic oligonucleotide
  misc_feature (1)..(1): 5′ biotin
atccagagtgacgcagcatttcatcgggtccaaaaggggctgctcgggattgcggatatggacacgt

(19) United States
(12) Patent Application Publication
(10) Pub. No.: US 2024/0011990 A1
(43) Pub. Date: Jan. 11, 2024

(54) VIRAL DETECTION SYSTEMS AND USES THEREOF

(71) Applicant: TRUSTEES OF BOSTON UNIVERSITY, Boston, MA (US)

(72) Inventors: Karthika SANKAR, Boston, MA (US); Scott SCHAUS, Boston, MA (US); James GALAGAN, Needham, MA (US); Catherine KLAPPERICH, Brookline, MA (US); John CONNOR, Newton, MA (US); Mark GRINSTAFF, Brookline, MA (US); Keith HEARON, Boston, MA (US)

(73) Assignee: TRUSTEES OF BOSTON UNIVERSITY, Boston, MA (US)

(21) Appl. No.: 18/016,497

(22) PCT Filed: Jul. 16, 2021
(86) PCT No.: PCT/US2021/042008

§ 371 Date: Jan. 17, 2023

Related U.S. Application Data
(60) Provisional application No. 63/084,814, filed on Sep. 29, 2020.
(60) Provisional application No. 63/053,048, filed on Jul. 17, 2020.

(51) Int. Cl. G01N 33/569; G01N 33/543; C12Q 1/00; G01N 33/53; G01N 27/327

(52) U.S. Cl. CPC G01N 33/56983; G01N 33/54386; C12Q 1/006; G01N 33/5308; G01N 27/3272; G01N 27/3276; G01N 27/3277; G01N 27/3278; G01N 2333/165; G01N 2333/11

(57) ABSTRACT

The systems, methods, devices and kits disclosed herein be used to determine the presence and/or level a target analyte(s) in a biological sample, wherein the analyte(s) is associated with a pathogenic (e.g., a viral antigen or whole virus) or otherwise altered physiological condition (e.g., pregnancy). In certain embodiments, the systems, methods, devices and kits provide one or more improved properties relative to the RT-PCT and lateral flow antibody assays known in the art for the detection of SARS-CoV-2, including but not limited to, assay time, ease of use, risk of infection, accuracy, specificity, selectivity, limit of detection of the assay, quantitative detection and the effect of common interferents to the sensor output, cost, simplicity or a combination thereof.

### CROSS-REFERENCE TO RELATED APPLICATIONS

This application claims benefit under 35 U.S.C. § 119(e) of U.S. Provisional Application No. 63/053,048 filed Jul. 17, 2020, and U.S. Provisional Application No. 63/084,814 filed Sep. 29, 2020, the contents of which are incorporated herein by reference in their entireties.

### GOVERNMENT SUPPORT

This invention was made with Government support under Grant No. AI151559, awarded by the National Institutes of Health, and Contract No. TTW-27-9661 awarded by the Department of Defense. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jul. 15, 2021, is named 701586-098060WOPT_SL.txt and is 63,151 bytes in size.

### TECHNICAL FIELD

Disclosed herein are systems, methods, devices and kits for detecting and monitoring a target analyte(s). In certain embodiments, the target analyte is associated with the result of pathological condition (e.g., a viral infection), while in others the target analyte is the pathologic agent (e.g.,) virus itself. In certain embodiments, disclosed are rapid quantitative point-of-care (POC) systems, single-user systems, or home use systems and methods for the detection of SARS-CoV-2, influenza, or other viruses, wherein the systems and methods incorporate a glucose oxidase-based amperometric sensor.

### BACKGROUND

Infectious disease continues to pose a significant public health and economic threat, including longstanding, emerging and reemerging pathogens. The coronavirus pandemic (SARS-CoV-2) is a devastating example, with interventions urgently sought. Irrespective of the coronavirus pandemic, the rate of infectious disease spread was already on the rise and demographic trends and climate change suggest that the problem may only grow worse.

Comprehensive testing is an important approach for identifying infected individuals for quarantine and/or early interventions. The current gold standard for SARS-CoV-2 testing is based on reverse transcriptase-polymerase chain reaction (RT-PCR) and relies on using respiratory samples to obtain viral nucleic acid, experienced personnel, extensive stocks of reagents, expensive equipment, and is riddled with high false negatives. In addition to RT-PCR having a high false negative rate, the experience of providing a respiratory sample (e.g., a nasal swab) can be uncomfortable and therefore, discourage testing. The result is that the quantity and value of RT-PCR testing for SARS-CoV-2 has been sub-optimal.

As an alternative, several lateral-flow immunoassays have been developed for the detection of SARS-CoV-2 antibodies. These tests look for the presence of three types of antibodies, namely IgG, IgM and IgA produced in the body as an immune response. However, these antibody-based assays are not useful as RT-PCR for early diagnosis of SARS-CoV-2 infection, as the body typically takes 5-10 days post-infection to produce these antibodies. Finally, antibody tests tend to suffer from sensitivity and specificity issues that result in a high false positive rate.

There remains a need to improve the quantity and value of testing for SARS-CoV-2 as well as testing for other viral pathological and altered physiological conditions.

### SUMMARY

The systems, methods, devices and kits disclosed herein can be used to determine the presence and/or level of target analyte(s) in a biological sample, e.g., wherein the analyte(s) is associated with a pathogenic or otherwise altered physiological condition due to a virus infection. In certain embodiments, the systems, methods, devices and kits provide one or more improved properties relative to the RT-PCR and lateral flow antibody assays known in the art, including but not limited to, assay time, ease of use, risk of infection, accuracy, specificity, selectivity, limit of detection of the assay, quantitative detection, and the effect of common interferents to the sensor output, cost, simplicity or a combination thereof.

In one aspect, a system is disclosed for detecting at least one target analyte in a biological sample, wherein the system comprises (i) at least a two binding agent assay, wherein the assay contains a first and second binding agent capable of creating a detectable complex with the at least one target analyte; and (ii) a detection device for detecting the detectable complex, wherein the detection device is a glucometer or glucose oxidase-based amperometric sensor and the biological sample is present in sweat, saliva, serum, mucus, or blood.

In one embodiment, the glucometer is a handheld portable glucose meter and includes a glucose sensor having a sensor output related to glucose in a biological sample on a test strip. In particular, the glucose sensor produces an output that is correlated with the presence or concentration of the target analyte(s), which is not glucose, within a biological sample.

In one embodiment, glucose oxidase catalyzes the oxidation of glucose to form hydrogen peroxide, which is then quantified by amperometric measurements (e.g. change in electrical current) through one or more electrodes. As the amount of glucose in the biological sample is in excess and, added for the detection, the amperometric quantification is for the target analyte e.g., SARS-CoV-2, H1N1, etc.

In one embodiment, the biological sample is saliva.

In other embodiments, the biological sample is urine, sweat, ocular fluid including aqueous humor, blood, fecal matter, sebum, respiratory droplets, semen, vaginal mucus, cerumen, epidermal cells, sputum, pleural effusion, cerebral fluid, or nasopharyngeal specimens.

In one embodiment, the biological sample is mixed with glucose.

In other embodiments, the biological sample is mixed with glucose at a concentration between 0.01 mM and 1 M.

In other embodiments, the biological sample is mixed with sucrose, fructose, maltose, galactose, cellulose, or any combination that includes an amylase or invertase at a concentration between 0.01 mM and 1 M (what is the solubility limit).

In one embodiment of the detectable complex for the target analyte, the first and second binding agents are selected from aptamers, antibodies, or proteins.

In one embodiment of the detectable complex for the target analyte, the first and second binding agent is selected from aptamers, antibodies, or proteins and the second binding agent are selected from aptamers, antibodies, or proteins which has been linked to an oxidase enzyme.

In a particular embodiment of the detectable complex for the target analyte, the first and second binding agent is each selected from aptamers, antibodies, or proteins and the second binding agent is selected from aptamers, antibodies, or proteins which are linked to glucose oxidase and produced from a fermentation process.

In a particular embodiment of the detectable complex for the target analyte, the first and second binding agent is each selected from aptamers, antibodies, or proteins and the second binding agent is selected from aptamers, antibodies, or proteins which has been linked to glucose oxidase, galactose oxidase, D-glucose:D-fructose oxidoreductase, and cellobiose oxidase.

In one embodiment of the detectable complex for the target analyte, the first and second binding agent is each selected from aptamers, antibodies, or proteins and the second binding agent is selected from aptamers, antibodies, or proteins which has been linked to a hydrogenase enzyme.

In a particular embodiment of the detectable complex for the target analyte, the first and second binding agent is each selected from aptamers, antibodies, or proteins and the second binding agent is selected from aptamers, antibodies, or proteins which has been linked to glucose dehydrogenase, glucose 6-phosphate dehydrogenase, fructose dehydrogenase, sucrose dehydrogenase, glucoside dehydrogenase, alcohol dehydrogenase, sorbitol dehydrogenase, lactate dehydrogenase, and malate dehydrogenase.

In a particular embodiment of the detectable complex for the target analyte, the first and second binding agents bind at a first and different second site on the target analyte, respectively.

In another embodiment of the detectable complex for the target analyte, the first and second binding agents bind the same site on the target analyte, respectively. As the site on the target analyte is present in multiple copies (>100), there are sufficient sites for the first and second binding agents.

In certain embodiments, the binding affinity of the first binding agent for the first site on the target analyte is greater than the binding affinity of the second binding agent for the second site on the target analyte.

In certain embodiments, the binding affinity of the first binding agent for the first site on the target analyte is similar to the binding affinity of the second binding agent for the second site on the target analyte.

In certain embodiments, the binding affinity of the first binding agent for the first site on the target analyte is weaker than the binding affinity of the second binding agent for the second site on the target analyte.

In one embodiment, the at least one target analyte is selected from virus or antibodies produced from a virus infection.

In a particular embodiment, at least one target analyte is a virus and more particularly, a coronavirus such as a betacoronavirus and even more particularly, SARS-CoV-2.

In one embodiment, the first and second binding agents bind different epitopes on the SARS-CoV-2 spike (S) protein. In a particular embodiment, at least one of the epitopes is within the receptor binding domain (RBD) of the S protein.

In one embodiment, one of the binding agents bind the SARS-CoV-2 spike (S) protein using the human angiotensin converting enzyme (ACE). In a particular embodiment, the ACE protein binds the receptor binding domain (RBD) of the S protein.

In a particular embodiment, at least one target analyte is a virus and more particularly, a coronavirus such as a betacoronavirus and even more particularly, SARS-CoV.

In one embodiment, the first and second binding agents bind different epitopes on the SARS-CoV spike (S) protein. In a particular embodiment, at least one of the epitopes is within the receptor binding domain (RBD) of the S1 protein.

In one embodiment, one of the binding agents binds the SARS-CoV-1 spike (S) protein using the human angiotensin converting enzyme (ACE). In a particular embodiment, the ACE protein binds the receptor binding domain (RBD) of the S protein.

In a particular embodiment, at least one target analyte is a virus and more particularly, a rhinovirus.

In one embodiment, the first and second binding agents bind one of the 4 possible capsid proteins of the rhinovirus.

In a particular embodiment, at least one target analyte is a virus and more particularly, common human coronaviruses, including types 229E, NL63, OC43, and HKU1.

In one embodiment, the first and second binding agents bind the spike protein, the membrane protein, the hemagglutinin protein, the envelope or envelope protein of common human coronaviruses.

In a particular embodiment, at least one target analyte is a virus and more particularly, respiratory syncytial virus (RSV), parainfluenza (PIV), H1N1, or herpesvirus.

In one embodiment, the first and second binding agents bind the fusion protein, the membrane protein, the hemagglutinin protein, the neuraminidase protein, the envelope, or envelope protein of respiratory syncytial virus (RSV) parainfluenza (PTV), or H1N1.

In a particular embodiment, at least one target analyte is a virus and more particularly, human metapneumovirus.

In one embodiment, the first and second binding agents bind the fusion protein, the SH protein, the matrix protein, the glycoprotein, the envelope, or envelope protein of human metapneumovirus.

In a particular embodiment, at least one target analyte is a virus and more particularly, human immunodeficiency virus (HIV).

In one embodiment, the first and second binding agents bind the MIHC protein, the p17 matrix protein, the gp120 docking glycoprotein, the gp41 transmembrane glycoprotein, the envelope, or envelope protein of human immunodeficiency virus (HIV).

In a particular embodiment, at least one target analyte is a virus and more particularly, Ebola virus.

In one embodiment, the first and second binding agents bind the glycoprotein, the matrix protein, the nucleoprotein, the envelope, or envelope protein of Ebola virus.

In a particular embodiment, at least one target analyte is a virus and more particularly, Marburg virus.

In one embodiment, the first and second binding agents bind the glycoprotein, the VP40 matrix protein, the nucleoprotein, the envelope, or envelope protein of Marburg virus.

In a particular embodiment, at least one target analyte is a virus and more particularly, Lassa virus.

In one embodiment, the first and second binding agents bind the glycoprotein 1, the glycoprotein 2, the large protein, the zinc protein, the stable signal peptide (SSP), the nucleoprotein, the envelope, or envelope protein of Lassa virus.

In one embodiment, the binding assay permits detection of more than one target analyte, e.g., more than one virus.

In another particular embodiment, the first binding agent is an aptamer, antibody, or protein bound to a test strip.

In another particular embodiment, the first binding agent is an aptamer, antibody, or protein bound to a test strip via a polymeric membrane located on the strip.

In another particular embodiment, the first binding agent is an aptamer, antibody, or protein bound to a test strip via a hydrophilic membrane such as a nitrocellulose membrane located on the strip.

In another particular embodiment, the first binding agent is an aptamer, antibody, or protein bound to a test strip via a hydrophilic membrane such as a nitrocellulose membrane which is placed directly above the electrode(s) or between two electrodes on the strip.

In another particular embodiment, the first binding agent is an aptamer, antibody, or protein bound to a test strip via a hydrophilic membrane such as a nitrocellulose membrane which is secured to the strip above the electrode(s) or between two electrodes.

In another particular embodiment, the first binding agent is an aptamer, antibody, or protein bound to a test strip via a hydrophilic membrane wherein the membrane also collects the biological sample(s) and provides a sink area to flow the sample from one location on the membrane to another.

In another particular embodiment, the first binding agent is an aptamer, antibody, or protein bound to a test strip via a hydrophilic membrane such as a nitrocellulose membrane wherein the membrane also collects the biological sample(s) and provides a sink area to flow the sample from one location on the membrane to another.

In another aspect, a system is disclosed for detecting at least one virus in a biological sample, wherein the system comprises: (i) a two binding agent assay, wherein the assay contains a first and second binding agent capable of creating a detectable complex with the at least one virus; and (ii) a detection device for detecting the detectable complex, wherein the detection device is a glucometer (also referred to herein as a glucose meter).

In one embodiment, the biological sample is saliva.

In other embodiments, the biological sample is urine, sweat, ocular fluid including aqueous humor, blood, fecal matter, sebum, respiratory droplets, semen, vaginal mucus, cerumen, epidermal cells, nasal sample, pleural effusion, cerebral spinal fluid, or nasopharyngeal specimens. In one embodiment, the biological sample is mixed with glucose.

In other embodiments, the biological sample is mixed with glucose at a concentration between 0.01 mM and 1M.

In other embodiments, the biological sample is mixed with sucrose, fructose, maltose, galactose, cellulose, or any combination that includes an amylase or invertase at a concentration between 0.01 mM and 1 M.

In one embodiment of the detectable complex for the target analyte, the first and second binding agents are selected from aptamers, antibodies, or proteins.

In one embodiment of the detectable complex for the target analyte, the first and second binding agent is selected from aptamers, antibodies, or proteins, and the second binding agent are selected from aptamers, antibodies, or proteins which has been linked to an oxidase enzyme.

In a particular embodiment of the detectable complex for the target analyte, the first and second binding agent is selected from aptamers, antibodies, or proteins, and the second binding agent are selected from aptamers, antibodies, or proteins linked to glucose oxidase and produced via a fermentation process.

In a particular embodiment of the detectable complex for the target analyte, the first and second binding agent is selected from aptamers, antibodies, or proteins, and the second binding agent are selected from aptamers, antibodies, or proteins which has been linked to glucose oxidase, galactose oxidase, D-glucose:D-fructose oxidoreductase, and cellobiose oxidase.

In one embodiment of the detectable complex for the target analyte, the first and second binding agent is selected from aptamers, antibodies, or proteins, and the second binding agent are selected from aptamers, antibodies, or proteins which has been linked to a hydrogenase enzyme.

In a particular embodiment of the detectable complex for the target analyte, the first and second binding agent is selected from aptamers, antibodies, or proteins, and the second binding agent are selected from aptamers, antibodies, or proteins which has been linked to glucose dehydrogenase, glucose 6-phosphate dehydrogenase, fructose dehydrogenase, sucrose dehydrogenase, glucoside dehydrogenase, alcohol dehydrogenase, sorbitol dehydrogenase, lactate dehydrogenase, and malate dehydrogenase.

In a particular embodiment of the detectable complex for the target analyte, the first and second binding agents bind at a first and different second site on the target analyte, respectively.

In another embodiment of the detectable complex for the target analyte, the first and second binding agents bind the same site on the target analyte, respectively. As the site on the target analyte is present in multiple copies (>100), there are sufficient sites for the first and second binding agents.

In certain embodiments, the binding affinity of the first binding agent for the first site on the target analyte is greater than the binding affinity of the second binding agent for the second site on the target analyte.

In certain embodiments, the binding affinity of the first binding agent for the first site on the target analyte is similar to the binding affinity of the second binding agent for the second site on the target analyte.

In certain embodiments, the binding affinity of the first binding agent for the first site on the target analyte is weaker than the binding affinity of the second binding agent for the second site on the target analyte.

In one embodiment, the binding assay permits the detection of more than one virus and in particular, (i) a coronavirus such as a betacoronavirus and even more particularly, SARS-CoV-2 and (ii) a respiratory virus and even more particularly, influenza.

In another aspect, a method for a diagnostic assessment is disclosed comprising: (i) collecting a biological sample from a subject, wherein the biological sample is not blood; (ii) adding the biological sample to a test strip in the presence of glucose, wherein the test strip contains a first and second binding agent capable of creating a detectable complex with at least one target analyte, if present in the biological sample, if present in the biological sample; (iii) introducing the test strip into a detection device (such as a glucometer); (iv) incubating the biological sample with the test strip; (v) detecting the level of detectable complex, if any, through a chemical reaction between glucose and glucose oxidase; and (vi) correlating the level of the detectable complex, if produced, with the quantity of the target analyte in the at least one biological sample, if any, thereby providing a diagnostic assessment.

In another aspect, a method for a diagnostic assessment is disclosed comprising: (i) collecting a biological sample from a subject, wherein the biological sample is not blood; (ii) adding the biological sample to a test strip in the presence of glucose, wherein the test strip contains a first and second binding agent capable of creating a detectable complex with at least one target analyte, if present in the biological sample, if present in the biological sample; (iii) incubating or not incubating the biological sample with the test strip; (iv) introducing the test strip into a detection device (such as a glucometer); (v) detecting the level of detectable complex, if any, through a chemical reaction between glucose and glucose oxidase; and (vi) correlating the level of the detectable complex, if produced, with the quantity of the target analyte in the at least one biological sample, if any, thereby providing a diagnostic assessment.

In another aspect, a method for a diagnostic assessment is disclosed comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) adding the biological sample to a test strip, wherein the test strip contains a first and second binding agent capable of creating a detectable complex with at least one target analyte in the biological sample, if present; (iv) introducing the test strip into a detection device such as a glucometer; (v) incubating the biological sample with the test strip; (vi) detecting the level of detectable complex, if any, through a chemical reaction between glucose and glucose oxidase; and (vii) correlating the level of the detectable complex, if produced, with the quantity of the target analyte in the at least one biological sample, if any, thereby providing a diagnostic assessment.

In another aspect, a method for diagnostic assessment is disclosed, comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) diluting the collected sample by 1×-100× in an aqueous solution/mixture in the presence of second binding agent; (iii) adding the biological sample and second binding agent to a test strip, wherein the test strip contains a first binding agent capable of creating a detectable complex with at least one target analyte in the biological sample, if present; (iv) incubating or not incubating the biological sample with the test strip; (v) introducing the test strip into a detection device; (vi) detecting the level of detectable complex, if any, through a chemical reaction between glucose and glucose oxidase; and (vii) correlating the level of the detectable complex, if produced, with the quantity of the target analyte in the at least one biological sample, if any, thereby providing a diagnostic assessment.

In another aspect, described herein is a method for a diagnostic assessment, comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) adding the biological sample to a test strip, wherein the test strip contains a first and second binding agent capable of creating a detectable complex with at least one target analyte in the biological sample, if present; (iii) incubating or not incubating the biological sample with the test strip; (iv) introducing the test strip into a detection device such as a glucometer; (v) detecting the level of detectable complex, if any, through a chemical reaction between glucose and glucose oxidase; and (vi) correlating the level of the detectable complex, if produced, with the quantity of the target analyte in the at least one biological sample, thereby providing a diagnostic assessment.

In another aspect, described herein is a method for a diagnostic assessment, comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) diluting the collected sample by 1×-100× in an aqueous solution/mixture in the presence of second binding agent; (iii) adding the biological sample and second binding agent to a test strip, wherein the test strip contains a first binding agent capable of creating a detectable complex with at least one target analyte in the biological sample, if present; (iv) incubating or not incubating the biological sample with the test strip; (v) introducing the test strip into a detection device such as a glucometer; (vi) detecting the level of detectable complex, if any, through a chemical reaction between glucose and glucose oxidase; and (vii) correlating the level of the detectable complex, if produced, with the quantity of the target analyte in the at least one biological sample, thereby providing a diagnostic assessment.

In another aspect, described herein is a method for a diagnostic assessment, comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) adding the biological sample to a test strip, wherein the test strip contains a first and second binding agent capable of creating a detectable complex with at least one target analyte in the biological sample, if present; (iii) incubating the biological sample with the test strip; (iv) introducing the test strip into a detection device such as a glucometer; (v) detecting the level of detectable complex, if any, through a chemical reaction between glucose and glucose oxidase; (vi) correlating the level of the detectable complex, if produced, with the quantity of the target analyte in the at least one biological sample, if any, thereby providing a diagnostic assessment; (vii) transmitting the diagnostic assessment or result to an electronic device, data base, or cloud server for subsequent review by a clinician or trained healthcare provider; and (viii) transmission of the diagnostic assessment or outcome to the individual who performed the method of diagnostic assessment.

In another aspect, described herein is a method for a diagnostic assessment, comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) diluting the collected sample by 1×-100× in an aqueous solution/mixture in the presence of second binding agent; (iii) adding the biological sample to a test strip, wherein the test strip contains a first binding agent capable of creating a detectable complex with at least one target analyte in the biological sample, if present; (iv) incubating the biological sample with the test strip; (v) introducing the test strip into a detection device such as a glucometer; (vi) detecting the level of detectable complex, if any, through a chemical reaction between glucose and glucose oxidase; (vii) correlating the level of the detectable complex, if produced, with the quantity of the target analyte in the at least one biological sample, if any, thereby providing a diagnostic assessment; (viii) transmitting the diagnostic assessment or result to an electronic device, data base, or cloud server for subsequent review by a clinician or trained healthcare provider; and (ix) transmission of the diagnostic assessment or outcome to the individual who performed the method of diagnostic assessment.

In another aspect, a test trip used for a biological assessment is disclosed comprising at least one of the following: (i) one or more planar or co-planar electrode made of carbon, iron, palladium, platinum or gold; (ii) an electrode coated with iron salts, such as ferrous ferrocyanide salts, as a mediator; (iii) an electrode coated with Prussian blue as a mediator; (iv) an n-electrode set-up where an electrode is a (semi-) conductive solid that interfaces with an electrolyte solution; (v) the electrode set-up includes a working electrode, a reference electrode, and a counter or auxiliary electrode; (vi) a two-electrode set-up that has the current and sense leads connected together, a working and working sense are connected to a working electrode and reference and counter are connected to a second auxiliary, counter, or quasi-/pseudo-reference electrode; (vii) a three electrode set-up, the reference lead is separated from the counter and connected to a third electrode most often positioned so that it is measuring a point very close to the working electrode that has both working and working sense leads attached; (viii) a four-electrode set-up with the working sense lead decoupled from the working electrode, in addition to the reference lead; and/or (ix) a zero resistance ammeter where the working and counter electrode leads are shorted together inside the strip so that there is zero net voltage dropped across the whole electrochemical cell.

The results obtained using the systems and methods described herein can be reported in a format selected from binary (e.g., yes/no), semi-quantitative (e.g., low, medium and high), or quantitative.

In another aspect, a test strip is disclosed comprising: (i) a substrate, at least one first and second binding agent and two or more electrodes; (ii) the substrate both first and second binding agents and two or more electrodes; (iii) at least one first and second binding agent and two or more electrodes; or (iv) both first and second binding agents and two or more electrodes.

In one embodiment, the test strip further comprises a test site, (i) wherein the test site contains the first binding agent; (ii) wherein the test site contains both the first and second binding agents; or (iii) wherein the test site contains the substrate and the first binding agent; or (iv) wherein the test site contains the substrate and both the first and second binding agents.

In another aspect, described herein is a test strip for use in the systems described herein, wherein the test strip comprises: (i) a substrate, at least one first and second binding agent and two or more electrodes; (ii) the substrate both first and second binding agents and two or more electrodes; (iii) at least one first and second binding agent and two or more electrodes; or (iv) both first and second binding agents and two or more electrodes.

In another aspect, a kit is disclosed comprising the test strip and optionally, directions for using the test strip. In certain embodiments, the kit further comprises a glucometer.

In another aspect, a localized or cloud-based software algorithm is disclosed that triggers electrochemical reactions in a detection system such that one or more detectable chemical species are the reaction product of a biological sample, test strip, and detection device.

In another aspect, described herein is a method comprising: (i) providing a biological sample from a subject, (ii) detecting the presence of a target analyte in the biological sample utilizing a system as described herein, wherein the target analyte is the SARS-CoV-2 virus or a component thereof, and (iii) optionally, treating the subject with a therapeutic agent (e.g., that is effective against SARS-CoV-2 virus).

In another aspect, described herein is a method comprising: (i) providing a biological sample from a subject, (ii) detecting the presence of a target analyte in the biological sample utilizing a system as described herein, wherein the target analyte is a CoV virus or a component thereof and (iii) optionally, treating the subject with a therapeutic agent.

In another aspect, a method for treatment is provided, comprising (e.g., the sequential steps): (i) determining, by (a) providing a biological sample from a subject, wherein the biological sample is saliva, and (b) detecting the presence of a target analyte in the biological sample using the system described herein, wherein the target analyte is the SARS-CoV-2 virus or a component thereof (e.g., S-1 protein); and (ii) treating the subject with a therapeutic agent selected from a small molecule or biologic agent (e.g., that is effective against SARS-CoV-2 virus).

In another aspect, described herein is a method comprising: (i) providing a biological sample from a subject, (ii) detecting the presence of a target analyte in the biological sample utilizing a system as described herein, wherein the target analyte is influenza virus or a component thereof and (iii) optionally, treating the subject with a therapeutic agent (e.g., that is effective against influenza virus).

In another aspect, described herein is a method comprising: (i) providing a biological sample from a subject, (ii) detecting the presence of a target analyte in the biological sample utilizing a system as described herein, wherein the target analyte is a hepatitis virus or a component thereof and (iii) optionally, treating the subject with a therapeutic agent (e.g., that is effective against hepatitis virus).

In another aspect, described herein is a system for detecting at least one target nucleic acid in a biological sample, wherein the system comprises (i) a sequence-specific endonuclease and guide nucleic acid that cleave a collateral nucleic acid upon specific binding of the target nucleic acid to the endonuclease and guide nucleic acid; (ii) a detection nucleic acid that is capable of creating a detectable complex with the cleaved collateral nucleic acid; and (iii) a detection device for detecting the detectable complex, wherein the detection device is an oxidase-based amperometric sensor and the biological sample is present in sweat, salvia, serum, mucus, or blood.

In some embodiments of any of the aspects, the sequence-specific endonuclease is a Cas enzyme.

In some embodiments of any of the aspects, the sequence-specific endonuclease is Cas12a or Cas13.

In some embodiments of any of the aspects, the guide nucleic acid is complementary or substantially complementary to at least a portion of the target nucleic acid.

In some embodiments of any of the aspects, the detection nucleic acid is complementary or substantially complementary to at least a portion of the cleaved collateral nucleic acid.

In some embodiments of any of the aspects, the detection nucleic acid hybridizes to the cleaved collateral nucleic acid.

In some embodiments of any of the aspects, the detection nucleic acid does not hybridize to the un-cleaved collateral nucleic acid.

In some embodiments of any of the aspects, the detection nucleic acid is linked to a test strip.

In some embodiments of any of the aspects, the collateral nucleic acid is linked to glucose oxidase.

In some embodiments of any of the aspects, the system further comprises an aptamer linked to glucose oxidase.

In some embodiments of any of the aspects, the aptamer specifically binds to at least a portion of the cleaved collateral nucleic acid.

In some embodiments of any of the aspects, the aptamer binds to a single-stranded portion of the cleaved collateral nucleic acid.

In some embodiments of any of the aspects, the aptamer binds to a double-stranded portion of the cleaved collateral nucleic acid hybridized to the detection nucleic acid.

In some embodiments of any of the aspects, the system further comprises an antibody linked to glucose oxidase.

In some embodiments of any of the aspects, the antibody specifically binds to at least a portion of the cleaved collateral nucleic acid.

In some embodiments of any of the aspects, the collateral nucleic acid is linked to an antibody that specifically binds glucose oxidase.

In some embodiments of any of the aspects, the collateral nucleic acid is linked to a first member of an affinity pair.

In some embodiments of any of the aspects, the system further comprises glucose oxidase linked to a second member of an affinity pair.

In some embodiments of any of the aspects, the first and second members of the affinity pair is selected from the group consisting of: a haptenic or antigenic compound in combination with a corresponding antibody or binding portion or fragment thereof, digoxigenin and anti-digoxigenin; mouse immunoglobulin and goat anti-mouse immunoglobulin; a non-immunological binding pair; biotin and avidin; biotin and streptavidin; a hormone and a hormone-binding protein; thyroxine and cortisol-hormone binding protein; a receptor and a receptor agonist; a receptor and a receptor antagonist; acetylcholine receptor and acetylcholine or an analog thereof, IgG and protein A; lectin and carbohydrate; an enzyme and an enzyme cofactor; an enzyme and an enzyme inhibitor; complementary oligonucleotide pairs capable of forming nucleic acid duplexes; and a first molecule that is negatively charged and a second molecule that is positively charged.

In some embodiments of any of the aspects, the first and second members of the affinity pair is streptavidin and biotin.

In some embodiments of any of the aspects, streptavidin is linked to the collateral nucleic acid, and biotin is linked to the glucose oxidase.

In some embodiments of any of the aspects, biotin is linked to the collateral nucleic acid, and streptavidin is linked to the glucose oxidase.

In some embodiments of any of the aspects, the target nucleic acid is a viral nucleic acid.

In another aspect, described herein is a method for detecting a target nucleic acid using a nucleic acid detection system as described herein, comprising: (i) collecting a biological sample from a subject, and optionally, extracting nucleic acid from the biological sample; (ii) contacting the biological sample with a sequence-specific endonuclease, guide nucleic acid, and a collateral nucleic acid, wherein such contacting results in cleavage of the collateral nucleic acid, if the target nucleic acid is present; (iii) adding the biological sample to a test strip in the presence of glucose, wherein the test strip comprises a detection nucleic acid that is capable of creating a detectable complex with the cleaved collateral nucleic acid, if present; (iv) incubating or not incubating the biological sample with the test strip; (v) introducing the test strip into a detection device; (vi) detecting the level of detectable complex, if any, through a chemical reaction between glucose and glucose oxidase; and (vii) correlating the level of the detectable complex, if produced, with the quantity of the target analyte in the at least one biological sample, thereby providing a diagnostic assessment.

In some embodiments of any of the aspects, the biological sample is saliva.

In some embodiments of any of the aspects, the detection device is a glucose meter.

In some embodiments of any of the aspects, the method further comprises (viii) transmitting the diagnostic assessment or result to an electronic device, data base, or cloud server for subsequent review by a clinician or trained healthcare provider; and (ix) providing the diagnostic assessment to the individual who performed the method of diagnostic assessment.

In some embodiments of any of the aspects, the individual is the subject.

In some embodiments of any of the aspects, the method further comprises (viii) recommending, instructing and/or administering one or more therapeutic regimes to the subject in response to the diagnostic assessment.

In another aspect, described herein is a test strip linked to a detection nucleic acid.

In another aspect, described herein is a kit comprising the test strip linked to a detection nucleic acid.

### BRIEF DESCRIPTION OF THE DRAWINGS

FIG. 1 depicts an exemplary repurposed glucometer-based SARS-CoV-2 sensor.

FIG. 2 depicts an exemplary test strip design and signal output when the test strip is put into the glucometer.

FIG. 3 is a schematic depicting the sequences of events that occur in glucose biosensor system. Glucose oxidation by GOx results in D-glucono-δ-lactone. H₂O₂ reduction at the Prussian Blue (PB) film is measured by electrons transferred from the working electrode.

FIG. 4 depicts a line graph (left panel) showing the current over time for different virion (e.g., H1N1) concentrations. A bar graph (right panel) shows the area under the curve of the current vs. time plot (see e.g., left panel) for different virion concentrations in buffer.

FIG. 5 depicts a schematic of the CoV test strip described herein for a glucometer. The labels (1)-(16) correspond to the different functional parts of the strip, and the labels (A)-(H) correspond to the layers of the strip. In one embodiment, the sensor strip comprises (bottom up): (A) a base substrate; (B) a conductive layer which includes three electrodes; (C) an insulating layer exposing only part of the electrode where the sample to be tested is dropped; (D) a reagent layer containing mediator for ease of exchange of electrons; (E) an adhesive layer; (F) a hydrophilic nitrocellulose membrane, with a proximal membrane containing aptamer to capture the antigen and freeze dried glucose, and the distal end is the paper sink (13); (G) freeze-dried Ab-GOx; and (H) a top layer

FIG. 6 depicts a line graph showing the current over time for human saliva spiked with different H1N1 virion concentrations and diluted tenfold.

FIG. 7 depicts a schematic of localized software algorithm used to trigger detectable chemical changes in a detection device.

FIG. 8A-8B depict schematics of a cloud-based software algorithm in an external server used to process data and trigger chemical changes in a detection device.

FIG. 9 shows the receptor binding domain (RBD) of the spike protein sequence of SARS-CoV-2 aligned with other related coronaviruses. Specifically shown is the sequence alignment for the interacting domain of SARS-CoV-2 SARS-CoV, and MERS-CoV (see e.g., SEQ ID NOs: 1-9). See e.g., W. Tai, et al., Cellular & Molecular Immunology, (2020) 17:613-620; the content of which is incorporated herein by reference in its entirety.

FIG. 10 depicts a line graph showing the current overtime graph in an assay with pre-incubation of H1N1 virus and Ab-GOx.

FIG. 11 depicts a line graph showing the current over time with VSV-CoV-2 pseudotyped virus.

FIG. 12 depicts a series of line graphs showing the current overtime with SARS-CoV-2 virus using: rabbit polyclonal antibody-GOx conjugate (left panel), or membrane antibody-GOx conjugate (right panel).

FIG. 13 depicts a line graph and bar graph measuring the cross-reactivity to H1N1 virus of the aptamers and antibody targeting SARS-CoV-2. Note that detected the signal from 10⁴ H1N1 virions was not significantly different from the signal detected from 0 virions.

FIG. 14 is a schematic showing the detection of one or two viruses. For detection of a single virus, the test strip has one aptamer (or antibody) for one virus on it. Urine, salvia, etc. is added to strip, and the strip is inserted into a glucometer. For detection of two viruses (e.g., Sars-CoV-2 and influenza A H1N1), one part of the test strip has an aptamer (or antibody) for one virus and the other part has an aptamer (or antibody) for second virus. Urine, salvia, etc., is added to strip, the strip is separated into two strips via the perforated dashed line, and one strip inserted into glucometer and read followed by removal of the first strip and insertion and reading of the second one.

FIG. 15A-15F is a series of schematics showing alternative designs for the detection device described herein (see e.g., Example 8). FIG. 15A depicts a nucleic acid detection using a collateral cleavage by endonuclease where in the collateral nucleic acid linked to glucose oxidase. FIG. 15B-15C depict a collateral cleavage nucleic acid detection wherein an aptamer, which binds to a single stranded (FIG. 15B) or double stranded (FIG. 15C) region of collateral nucleic acid, is linked to glucose oxidase. FIG. 15D depicts nucleic acid detection wherein an antibody that binds the collateral nucleic acid is linked to glucose oxidase. FIG. 15E depicts nucleic acid detection wherein the collateral nucleic acid is linked to an antibody that binds specifically to glucose oxidase. FIG. 15F depicts nucleic acid detection wherein the collateral nucleic acid is linked to an affinity pair, with one member of the pair linked to glucose oxidase.

FIG. 16 depicts an example process and an example of an overview of a system according to some embodiments of the present disclosure. The top half of FIG. 16 is a flowchart showing an example process for detecting a target analyte in sample using a test strip and detection device as described herein. In some embodiments of any of the aspects, a test sample 110 is received (for example from a subject 100). Additional samples can include negative control(s) 111 and positive control(s) 112. In some embodiments of any of the aspects, the samples are optionally processed 120 (e.g., protein or nucleic acid extraction; e.g., dilution). The biological sample is then added to a test strip 135 in the presence of glucose 130. The test can comprise at least one of the detection reagents as described herein (e.g., antibody, aptamer, detection nucleic acid). The test strip is then introduced into a detection device 140 (e.g., a glucometer). The bottom half of FIG. 16 shows an example of an overview of a system according to some embodiments of the present disclosure. The test strip is input into detection device 150, which is part of a system that includes a network 160, a computing device 170, a display 175, a server 180, and/or a database 185.

### DETAILED DESCRIPTION

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used herein, the singular forms “a,” “an,” and “the” include plural referents unless the context clearly dictates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

The term “about” as used herein in connection with any and all values (including lower and upper ends of numerical ranges) refers to any value having an acceptable range of deviation of ±0.5% to ±20% (and values there between, e.g., ±1%, ±1.5%, ±2%, ±2.5%, ±3%, ±3.5%, ±4%, ±4.5%, ±5%, ±5.5%, ±6%, ±6.5%, ±7%, ±7.5%, ±8%, ±8.5%, ±9%, ±9.5%, +10%, ±10.5%, ±11%, ±11.5%, ±12%, ±12.5%, +13, ±13.5%, ±14%, ±14.5%, ±15%, ±15.5%, ±16%, ±16.5%, ±17%, ±17.5%, ±18%, ±18.5%, ±19%, ±19.5%, and +20%). Other than in the operating examples, or where otherwise indicated, all numbers expressing quantities of ingredients or reaction conditions used herein should be understood as modified in all instances by the term “about.” The term “about” when used in connection with percentages can mean ±1%, or ±0.5%-20% as outlined above.

The term “affinity” as used herein refers to a measure of the strength of the binding of between a target molecule and a binding agent. Affinity is typically expressed by a dissociation constant (Kd). Any Kd greater than about 10⁻⁶ M is generally considered to indicate nonspecific binding.

The term “amperometric” as used herein refers to a chemical titration in which the measurement of the electric current flowing under an applied potential difference between two electrodes in a solution is used for detecting the end point.

The term “analyte” as used herein is a broad term used to refer to a substance or chemical constituent in a fluid such as a biological fluid. Analytes can include naturally occurring substances, artificial substances, metabolites, and/or reaction products. The analyte can be naturally present in the biological fluid or endogenous; for example, a metabolic product, a hormone, an antigen, an antibody, and the like. Alternatively, the analyte can be introduced into the body or exogenous.

The term “antibody” or “immunoglobulin,” as used interchangeably herein, includes whole antibodies and any antigen binding fragment (antigen-binding portion) or single chain cognates thereof. An “antibody” comprises at least one heavy (H) chain and one light (L) chain. In naturally occurring IgGs, for example, these heavy and light chains are inter-connected by disulfide bonds and there are two paired heavy and light chains, these two also inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR) or Joining (J) regions (JH or JL in heavy and light chains respectively). Each VH and VL is composed of three CDRs three FRs and a J domain, arranged from amino-terminus to carboxy-terminus in the following order: FR1, CDR1, FR2, CDR2, FR3, CDR3, J. The variable regions of the heavy and light chains bind with an antigen. The constant regions of the antibodies may mediate the binding of the immunoglobulin to host tissues or factors, including various cells of the immune system (e.g., effector cells) or humoral factors such as the first component (Clq) of the classical complement system. The term “antibody” is used herein in the broadest sense and encompasses various antibody structures, including but not limited to monoclonal antibodies, polyclonal antibodies, multi-specific antibodies (e.g., bispecific antibodies), and antibody fragments, so long as they exhibit the desired antigen-binding activity.

The term “antigen” as used herein refers to an entity (e.g., a proteinaceous entity or peptide) to which an antibody binds. In certain embodiments, the antigen is a coronavirus protein (e.g., a spike protein), or a derivative, fragment, analog, homolog or ortholog thereof, serves as the antigen in the systems and methods disclosed herein.

The term “antigen-binding region” refers to that portion of a binding agent that (e.g., antibody, aptamer) that interact with a target molecule (e.g., an antigen) and confer on the binding agents its specificity and affinity for the target molecule.

The term “anti-viral drug” as used herein refers broadly to any anti-infective drug or therapy used to treat or ameliorate a viral infection in a subject.

The term “aptamer” as used herein refers to an oligonucleotide (DNA or RNA) that can conform in three-dimensions to bind another molecule with high affinity in the nanomolar and subnanomolar range. Exemplary nucleic acid molecules or polynucleotides comprising such aptamers include, but are not limited to, either D- or L-nucleic acids, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a beta-D-ribo configuration, alpha-LNA having an alpha-L-ribo configuration (a diastereomer of LNA), 2′-amino-LNA having a 2′-amino functionalization, and 2′-amino-alpha-LNA having a 2′-amino functionalization) or hybrids thereof. Aptamers can be linked to other molecules, including small molecules, proteins, nucleic acids, and even cells, tissues and organisms (e.g., whole virus) and may be monovalent or multivalent. Aptamers for use in the disclosed embodiments may be obtained by selection from a large random sequence library, using methods well known in the art, such as Synthetic Evolution of Ligands by Exponential Enrichment (SELEX).

The term “binding agent” as used herein refers a molecule that binds to a cognate ligand with high affinity and high specificity. A binding agent is typically used to identify the presence of its cognate ligand and can be detectably labeled to allow identification. An “X binding agent” means a molecule that binds to “X” with high affinity and high specificity. Examples of “X” binding agents include, e.g., an aptamer, an antibody, a receptor ligand, or a molecular imprinted polymer.

The term “binding pair” as used herein refers to a pair of molecules that bind to each other with high affinity and specificity. A “binding pair member” refers to one molecule of a binding pair. For example, streptavidin and biotin are binding pair members that non-covalently bind with each other. Additional non-limiting examples of first and second members of a binding pair (also referred to as an affinity pair) include: a haptenic or antigenic compound in combination with a corresponding antibody or binding portion or fragment thereof; digoxigenin and anti-digoxigenin; mouse immunoglobulin and goat anti-mouse immunoglobulin; a non-immunological binding pair; biotin and avidin; biotin and streptavidin; a hormone and a hormone-binding protein; thyroxine and cortisol-hormone binding protein; a receptor and a receptor agonist; a receptor and a receptor antagonist; acetylcholine receptor and acetylcholine or an analog thereof: IgG and protein A; lectin and carbohydrate; an enzyme and an enzyme cofactor; an enzyme and an enzyme inhibitor; complementary oligonucleotide pairs capable of forming nucleic acid duplexes; and a first molecule that is negatively charged and a second molecule that is positively charged.

The term “sample” or “test sample” as used herein denotes a sample taken or isolated from a biological organism, e.g., a blood or plasma sample from a subject. In one embodiment, the term “biological sample” as used herein refers to saliva. In other embodiments, the biological sample is urine, sweat, ocular fluid including aqueous humor, blood, fecal matter, sebum, respiratory droplets, semen, vaginal mucus, cerumen, epidermal cells, sputum, pleural effusion, cerebral fluid, or nasopharyngeal specimens. Additional exemplary biological samples include, but are not limited to, a biopsy; a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample, etc. The term also includes a mixture of the above-mentioned samples. The term “test sample” also includes untreated or pretreated (or pre-processed) biological samples. In some embodiments of any of the aspects, a test sample can comprise cells from a subject. The test sample can be obtained by removing a sample from a subject, but can also be accomplished by using a previously isolated sample (e.g. isolated at a prior time point by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, homogenization, sonication, filtration, thawing, purification, and any combinations thereof. In some embodiments of any of the aspects, the test sample can be treated with a chemical and/or biological reagent. Chemical and/or biological reagents can be employed, for example, to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived there from and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes, without limitation, cells in culture, cell supernatants, cell lysates, tissue, peripheral blood, serum, plasma, urine, cerebral spinal fluid, biological fluid, and tissue samples. The sample may be pretreated as necessary by dilution in an appropriate buffer solution or concentrated, if desired. Any of a number of standard aqueous buffer solutions, employing one of a variety of buffers, such as phosphate, Tris, or the like, preferably at physiological pH can be used. Biological samples can be derived from patients using well known techniques such as venipuncture, lumbar puncture, fluid sample such as saliva or urine, or tissue biopsy and the like. In certain embodiments, the sample is a body sample from any animal, in one embodiment it is from a mammal, in one embodiment from a human subject and in another embodiment, a non-human animal (e.g., an insect or bat).

The term “binding affinity” as used herein refers to the tendency of a binding agent to bind or not bind a target and describes the measure of the strength of the binding or affinity of the binding agent to bind the target molecule.

The term “capture reagent” as used herein refers to a reagent capable of binding and capturing a target molecule in a sample. Typically, the capture reagent is immobilized or immobilizable. In a sandwich immunoassay, the capture reagent may be an aptamer or antibody, for example.

The term “chronoamperometry” as used herein refers to an electrochemical measuring technique used for electrochemical analysis or for the determination of the kinetics and mechanism of electrode reactions. A fast-rising potential pulse is enforced on the working (or reference) electrode of an electrochemical cell and the current flowing through this electrode is measured as a function of time.

The term “complex” as used herein refers to an entity comprising more than one molecule which is bound or is in association with at least one other molecule, for example by a chemical association. Hence the term “matrix-aptamer-target molecule complex” relates to an association between the matrix, aptamer, and the target molecule. The term “biotinylated second binding agent streptavidin (or b-binding agent-SA) complex” relates to an association between biotin, a second binding agent, and streptavidin.

The term “correlated with” or “associated with” refer to the levels of an analyte or a fragment thereof in a biological sample of a subject that has a statistically significant correlation with a physiologic state, e.g., disease status or extent of the disease, response to treatment, and survival. The strength of the correlation between levels of an analyte or a fragment thereof and the presence or absence of a particular physiologic state may be determined by a statistical test of significance.

The term “statistically significant” or “significantly” refers to statistical significance and generally means a two standard deviation (2SD) or greater difference.

The term “cross-reactivity”, as used herein, refers to the ability of a binding agent (e.g., aptamer, antibody) directed against one target molecule to successfully bind with another, different molecule, i.e., a non-target molecule. The degree of cross-reactivity may vary. In certain embodiments, the target molecule and non-target molecule share a common epitope, i.e., a feature highly conserved across species.

The term “cut point”, as used herein, refers to threshold value used to distinguish between a negative and a positive response in the assay. It is a constant value, determined statistically by analyzing assay responses of a set of drug-naïve diseased human samples.

The term “detectable label” refers to a moiety, molecule or a compound or a group of molecules or a group of compounds associated with a binding agent and is used to identify the binding agent. Signals from the detectable label may be detected by various means and will depend on the nature of the detectable label. Detectable labels may be isotopes, fluorescent moieties, colored substances, enzymes, enzyme substrates, and the like. Examples of means to detect detectable label include but are not limited to spectroscopic, photochemical, biochemical, immunochemical, electromagnetic, radiochemical, or chemical means, such as fluorescence, chemifluorescence, chemiluminescence, or any other appropriate means.

The term “dropcasting” refers to a method in which a thin solid film is formed by dropping a solution onto a flat surface followed by evaporation of the solution.

The term “electrochemical system”, as used herein, refers to a system that determines the presence and/or quantity of a redox analyte through measurements of electrical signal in a solution between a working electrode and a counter electrode, such as induced by a redox reaction or electrical potential from the release or absorption of ions. The redox reaction refers to the loss of electrons (oxidation) or gain of electrons (reduction) that a material undergoes during electrical stimulation such as applying a potential. Redox reactions take place at the working electrode, and which, for chemical detection, is typically constructed from an inert material such as platinum or carbon. The potential of the working electrode is measured against a reference electrode, which is typically a stable, well-behaved electrochemical half-cell such as silver/silver chloride. The electrochemical system can be used to support many different techniques for determining the presence and/or concentration of the target biomolecules including, but not limited to, various types of voltammetry, amperometry, potentiometry, coulometry, conductometry, and conductimetry such as AC voltammetry, differential pulse voltammetry, square wave voltammetry, electrochemical impedance spectroscopy, anodic stripping voltammetry, cyclic voltammetry, and fast scan cyclic voltammetry. The electrochemical system may further include one or more negative control electrode, and positive control electrode. In the context of the present invention, a single electrochemical system may be used to quantify more than one type of analyte.

The term “epitope” or “antigenic determinant” are used interchangeably herein and refer to that portion of a molecule such as an antigen capable of being recognized and specifically bound by a particular binding agent (e.g., antibody or aptamer). When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation. An antigenic determinant can compete with the intact antigen (i.e., the “immunogen” used to elicit the immune response) for binding to an antibody.

The term “false negative” as used herein refers to a sample incorrectly identified not containing one or more analytes, e.g., viruses.

The term “false positive” as used herein refers to a sample incorrectly identified as containing one or more analysts, e.g., viruses.

The term “fragment”, as used herein, refers to a polypeptide or a polynucleotide having a sequence length of 1 to n−1, relative to a full-length polypeptide or polynucleotide (length is n). The length of the fragment can be appropriately changed according to the purpose thereof. Examples of a lower limit of the length thereof, in the case of a polypeptide, include 3, 4, 5, 6, 7, 8, 9, 10, 15, 20, 25, 30, 40, 50 and more amino acids, and a length represented by an integer which is not specifically listed herein (e.g. 11) can also be proper as a lower limit. In addition, in the case of a polynucleotide, examples of a lower limit of the length thereof include 5, 6, 7, 8, 9, 10, 15, 20, 25, 30, 40, 50, 75, 100, 200, 300, 400, 500, 600, 700, 800, 900, 1000 and more nucleotides, and a length represented by an integer which is not specifically listed herein (e.g., 11) can also be proper as a lower limit.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity according to the assays described below herein. A functional fragment can comprise conservative substitutions of the sequences disclosed herein.

The term “glucometer” has used herein refers to a medical device commonly used by diabetic patients for self-monitoring of blood glucose levels. Many glucometers use an electrochemical method, based on test media such as test strips. Test strips are a consumable element containing chemicals that, in the context of diabetes monitoring, react with glucose in a drop of blood used for each measurement. Specifically, a chemical reaction is produced and the meter reads the level of glucose expressed in mg/dl or mmol/l. The glucometer is usually portable and is used at home although professional glucometers are known.

The term “glucose” as used herein refers to a monosaccharide, common hexose sugar.

The term “high affinity” as used herein refers to binding affinity of at least 10⁻⁸ M, between about 10⁻⁸ and about 10⁻¹², or more particularly, about 10⁻⁸ M, about 10⁻⁹ M; about 10⁻¹⁰ M, about 10⁻¹¹ M, or about 10⁻¹² M.

The terms “isolated”, “purified” or “biologically pure”, as used herein, refer to material that is substantially or essentially free from components which normally accompany it as found in its native state. Purity and homogeneity are typically determined using analytical chemistry techniques such as polyacrylamide gel electrophoresis or high-performance liquid chromatography. A protein that is the predominant species present in a preparation is substantially purified.

The term “K_{D}” as used herein refers to the equilibrium dissociation constant of a particular binding agent-target molecule interaction.

The terms “measuring” and “determining” are used interchangeably throughout and refer to methods which include obtaining a patient sample and/or detecting the level of a biomarker(s) in a biological sample. In one embodiment, the terms refer to obtaining a patient sample and detecting the level of one or more biomarkers in the sample. In another embodiment, the terms “measuring” and “determining” mean detecting the level of one or more biomarkers in a biologic sample. The term “measuring” is also used interchangeably throughout with the term “detecting.”

The term “molecule” as used herein is used broadly to refer to natural, synthetic or semi-synthetic molecules or compounds.

The term “monoclonal antibody” as used herein refers to an antibody obtained from a population of substantially homogeneous antibodies, i.e., the individual antibodies comprising the population are identical except for possible naturally occurring mutations that can be present in minor amounts.

The term “mutation” refers to a change in the amino acid sequence of a native protein. Mutations can be described by using the native sequence and then identifying the specific acid that have been changed. A “mutant” or “variant” refers to the protein that contains the mutation. A full-length mutant sequence refers to the full amino acid sequence of the mutant protein, instead of describing the mutant as the amino acids that are different from the native protein.

The term “native protein” refers to a protein that is in its native or natural state and unaltered by any denaturing agent such as heat, chemical mutation or enzymatic reactions.

The term “non-target molecule” as used herein refers to a molecule that is not a biomarker of interest. In particular, a non-target molecule may be a molecule structurally similar to biomarker(s) of interest.

The term “pathogen” means any disease-producing agent including, but not limited to, a virus or bacterium or other microorganism. Replicating pathogens (e.g., viruses and bacteria) are organisms that cause disease by using the body's resources to replicate while largely avoiding the body's immune response.

The term “point of care testing” or “POCT” is used herein to refer to biological specimens assayed at or near the patient with the assumption that test results will be available instantly or in a very short timeframe to assist caregivers with immediate diagnosis and/or clinical intervention. See e.g., Ehrmeyer S S et al. (2007) Clin Chem Lab Med 45: 766-773. The term is not intended to be limited to patients and home use, but inclusive of a variety of setting (e.g., communities, clinics, peripheral laboratories and hospitals) and users (e.g. technicians and caregivers). Depending on the setting and the user, the purpose of POC testing may vary—from triage and referral, to diagnosis, treatment, and monitoring.

The term “potentiostat” as used herein is a broad term and is used in its ordinary sense, including, without limitation, an electrical system that controls the potential between the working and reference electrodes of a three-electrode cell at a preset value. It forces whatever current is necessary to flow between the working and counter electrodes to keep the desired potential, as long as the needed cell voltage and current do not exceed the compliance limits of the potentiostat.

The term “pre-determined threshold (value)” means the threshold numeric value at which a classifier gives the desirable balance between (the cost of) false negatives and false positives. In some embodiments, “predetermined threshold” is statistically (and clinically) determined, refined, adjusted and/or confirmed through, on, or based on, a clinical study and analyses of outcome thereof (collectively, “clinical data”), and/or a preclinical or non-clinical study (collectively, “non-clinical data”), in order to minimize undesirable effects of false positives and false negatives.

The terms “prevent”, “preventing” or “prevention” refer to inhibition of manifestation of a pathologic condition, e.g., symptoms or indications of pathology, such as symptoms or indications of a viral infection.

The term “processor” as used herein is used broadly to refer to a programmable or non-programmable processing device, such as a microprocessor, microcontroller, application-specific integrated circuits (ASICS), programmable logic devices (PLDs), field-programmable gate arrays (FPGAs), etc. The term “processor” may also include multiple processing devices working in conjunction with one another.

The term “point mutation”, as used herein, refers to the engineering of a polynucleotide that results in the expression of an amino acid sequence that differs from the non-engineered amino acid sequence in the substitution or exchange, deletion or insertion of one or more single (nonconsecutive) or doublets of amino acids for different amino acids.

The words “protein”, “peptide”, and “polypeptide” are used interchangeably to denote an amino acid polymer or a set of two or more interacting or bound amino acid polymers, e.g., connected to each other by peptide bonds between the alpha-amino and carboxy groups of adjacent residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The term “reference value” as used herein can be a “threshold value” or a “cut-off value”. Typically, a “threshold value” or “cut-off value” can be determined experimentally, empirically, or theoretically.

The term “risk” as used herein refers to the probability that an event will occur over a specific time period, e.g., as in the conversion to Covid-19 positive test results, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no conversion. Alternative continuous measures, which may be assessed in the context of the present invention, include time to conversion risk reduction ratios.

The term “selectivity” as used herein refers to the ability a system or method to discriminate a particular analyte in a complex mixture without interference from other components.

The term “sensor” as used herein refers to a means used to detect an analyte. A “sensor system” includes, for example, elements, structures and architectures intended to facilitate sensor use and function. Sensor systems can include, for example, compositions such as those having selected material properties, as well as electronic components such as elements and devices used in signal detection and analysis (e.g., current detectors, monitors, processors and the like).

The term “specific binding”, “specifically binds,” “selective binding,” and “selectively binds” mean that a binding agent (e.g., antibody, aptamer) exhibits appreciable affinity for a target molecule and, generally, does not exhibit significant cross-reactivity with non-target molecules, which in certain embodiment means having an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis or surface plasmon resonance.

Specifically, as used herein, the term “specific binding” refers to a chemical or physical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The term “sensitivity” as used herein refers to proportion of positives that are correctly identified (e.g., the percentage of SARS-CoV-2 positive people that are identified by a system or method). In a highly sensitive system or method, false negatives are limited.

The term “specificity” as used herein refers the proportion of negatives that are correctly identified (e.g., the percentage of people who are correctly identified as not being infected with SARS-CoV-2 by a system or method). In a highly specific system or method, false positives are limited.

The term “screen printing” as used herein refers to a technique in which electrochemical measurement devices are manufactured by printing different types of ink on plastic or ceramic substrates, allowing quick in-situ analysis with high reproducibility, sensitivity, and accuracy. The composition of the different inks (e.g., carbon, silver, gold, platinum) used in the manufacture of the electrode can determine its selectivity and sensitivity. Screen printing permits the reproducible production of high-quality disposable electrodes at low cost. Other printing methods or other methods to form the electrodes are known in the art.

The term “subject” refers to a mammal, such as a human. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of, for example, infectious disease. A subject can be male or female.

A subject can be one who has been previously diagnosed with or identified as suffering from or having a condition in need of treatment of a disease or disorder as described herein (e.g., an infectious disease) or one or more complications related to such a condition, and optionally, have already undergone treatment for a disease or disorder as described herein (e.g., an infectious disease) or the one or more complications related to a disease or disorder as described herein (e.g., an infectious disease). Alternatively, a subject can also be one who has not been previously diagnosed as having a disease or disorder as described herein (e.g., an infectious disease) or one or more complications related to a disease or disorder as described herein (e.g., an infectious disease). For example, a subject can be one who exhibits one or more risk factors for a disease or disorder as described herein (e.g., an infectious disease) or one or more complications related to a disease or disorder as described herein (e.g., an infectious disease) or a subject who does not exhibit risk factors.

A “subject in need” of treatment for a particular condition can be a subject having that condition, diagnosed as having that condition, or at risk of developing that condition.

The term “system noise” as used herein refers to without limitation, unwanted electronic or diffusion-related noise which can include Gaussian, motion-related, flicker, kinetic, or other white noise, for example.

The term “target molecule” as used herein refers to a molecule which may be found in a tested sample and which is capable of binding to a binding agent.

As used herein, the terms “treat,” “treatment,” or “treating” refer to therapeutic treatments, wherein the object includes preventing, inhibiting, alleviating, reversing, ameliorating, slowing down, or stopping the progression or severity of a condition(s) and symptom(s) associated a with disorder(s) or disease(s), e.g. an infectious disease such as COVID-19. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The term “therapeutically effective amount” as used herein refers to that amount of active compound or pharmaceutical agent (e.g., an anti-viral drug) that elicits the biological or medicinal response in a subject that is being sought by a researcher, veterinarian, medical doctor, or other clinician, which includes preventing, ameliorating or alleviating the symptoms of the disease or disorder being treated. Methods are known in the art for determining therapeutically effective doses for the instant pharmaceutical composition.

The term “two binding agent assay” refers to an assay wherein the target molecule attached to the first binding agent bound to the matrix is further incubated in the presence of a second binding agent associated with a chemical reactive group.

The term “variant” as used herein is a relative term that describes the relationship between a particular polypeptide of interest and a “parent” or “reference” polypeptide to which its sequence is being compared. A polypeptide of interest is considered to be a “variant” of a parent or reference polypeptide if the polypeptide of interest has an amino acid sequence that is identical to that of the parent but for a small number of sequence alterations at particular positions. Variants include, for example, substitutional, insertional or deletion variant. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide. In a particular embodiment, a variant is a viral protein (e.g., a spike protein) that is similar to a reference viral protein, particularly in its function, but have mutations in their amino acid sequence that make them different in sequence from the wild-type viral protein at one or more positions.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. targeting binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of several different computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

The term “wild-type”, as used herein, refers to a native full-length form of a protein or nucleic acid, as is found in nature. The term full length native protein sequence, as used herein, refers to the amino acid sequence found in the full-length native protein. The wild-type protein may be obtained, for example, from a biological sample.

For any method disclosed herein that includes discrete steps, the steps may be conducted in any feasible order. And, as appropriate, any combination of two or more steps may be conducted simultaneously.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acids can include a guide nucleic acid, a collateral nucleic acid, and/or a detection nucleic acid. Suitable DNA can include, e.g., viral DNA, genomic DNA or cDNA. Suitable RNA can include, e.g., mRNA or viral RNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (e.g., mRNA) or antisense RNA derived from a nucleic acid fragment or fragments and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” refers to the nucleic acid sequence which is transcribed (DNA) to RNA in vitro or in vivo when operably linked to appropriate regulatory sequences. The gene may or may not include regions preceding and following a coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having an infectious disease (e.g., COVID-19), as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell is typically still referred to as “engineered” even though the actual manipulation was performed on a prior entity.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can be a level of the target molecule in a population of subjects who do not have or are not diagnosed as having, and/or do not exhibit signs or symptoms of a disease or disorder as described herein (e.g., an infectious disease, such as COVID-19). In some embodiments of any of the aspects, the reference can also be a level of expression of the target molecule in a control sample, a pooled sample of control individuals or a numeric value or range of values based on the same. In some embodiments of any of the aspects, the reference can be the level of a target molecule in a sample obtained from the same subject at an earlier point in time, e.g., the methods described herein can be used to determine if a subject's sensitivity or response to a given therapy is changing overtime.

In some embodiments, the reference level can be the level in a sample of similar cell type, sample type, sample processing, and/or obtained from a subject of similar age, sex and other demographic parameters as the sample/subject for which the level of the target analyte is to be determined. In some embodiments, the test sample and control reference sample are of the same type, that is, obtained from the same biological source, and comprising the same composition, e.g. the same number and type of cells.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

In some embodiments, a system for detecting one or more target analytes in a biological sample is disclosed. The system includes a binding assay for analysis of a biological sample other than blood and a detection means, wherein the detection means is a glucometer or similar device to measure or detect glucose. In certain embodiments, the system is an electrochemical system.

In one aspect, a system is disclosed for detecting at least target analyte in a biological sample, wherein the system comprises (a) a two binding agent assay, wherein the assay contains a first and second binding agent capable of creating a detectable complex with the at least one target analyte and (ii) a detection device for detecting the detectable complex, wherein the detection device is a glucometer and the biological sample is not blood.

In another aspect, a system is disclosed for detecting at least one pathogen (e.g., virus) in a biological sample, wherein the system comprises (a) a two binding agent assay, wherein the assay contains a first and second binding agent capable of creating a detectable complex with the at least one pathogen and (ii) a detection device for detecting the detectable complex, wherein the detection device is a glucometer. In certain embodiments, the biological sample is saliva.

In one embodiment, the system provides a Yes/No result. In other embodiments, the system provides a semi-quantitative or quantitative result (e.g., levels of analyte per volume such as number of viral copies per volume).

In certain embodiments, the first binding agent is a capture reagent immobilized to a test strip and the second binding agent is a detectable binding agent, where binding of the first and second binding agent to the target analyte (e.g., viral antigen or virus) results in a detectable complex.

In certain embodiments, the second binding agent is a binding agent-glucose oxidase (Ab-GOx) conjugate, wherein the glucometer provides an electrochemical signal that indicates the presence of the target analyte and/or correlates with the quantity of the target analyte present in the biological sample, wherein the biological sample is not blood.

In certain embodiments, the second binding agent is a binding agent-glucose oxidase (Ab-GOx) conjugate, wherein the glucometer provides an electrochemical signal that indicates the presence of the target analyte and/or correlates with the quantity of the target analyte present in the biological sample, wherein the biological sample is urine, sweat, ocular fluid including aqueous humor, blood, fecal matter, sebum, respiratory droplets, semen, vaginal mucus, cerumen, epidermal cells, nasal sample, pleural effusion, cerebral spinal fluid, or nasopharyngeal specimens.

In certain embodiments, the second binding agent is a binding agent-glucose oxidase (Ab-GOx) conjugate, wherein the glucometer provides an electrochemical signal that indicates the presence of the target analyte and/or correlates with the quantity of the target virus present in the biological sample, wherein the biological sample is not blood.

In certain embodiments, the second binding agent is a binding agent-glucose oxidase (Ab-GOx) conjugate, wherein the glucometer provides an electrochemical signal that indicates the presence of the target analyte and/or correlates with the quantity of the target virus present in the biological sample, wherein the biological sample is urine, sweat, ocular fluid including aqueous humor, blood, fecal matter, sebum, respiratory droplets, semen, vaginal mucus, cerumen, epidermal cells, or nasopharyngeal specimens.

In one embodiment, the biological sample is mixed with glucose.

In other embodiments, the biological sample is mixed with glucose at a concentration between 0.01 mM and 1 M.

In other embodiments, the biological sample is mixed with sucrose, fructose, maltose, galactose, cellulose, or any combination that includes an amylase or invertase, at a concentration between 0.01 mM and 1 M. In some embodiments of any of the aspects, the concentration of the glucose, sucrose, fructose, maltose, galactose, or cellulose is at least 0.01 mM, at least 0.02 mM, at least 0.03 mM, at least 0.04 mM, at least 0.05 mM, at least 0.06 mM, at least 0.07 mM, at least 0.08 mM, at least 0.09 mM, at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, or at least 1.0 mM or more.

In one embodiment of the detectable complex for the target analyte, the first and second binding agents are selected from aptamers, antibodies, or proteins or combinations thereof.

In one embodiment of the detectable complex for the target analyte, the first and second binding agent is selected from aptamers, antibodies, or proteins, and the second binding agent is selected from aptamers, antibodies, or proteins, in each case linked to an oxidase enzyme.

In a particular embodiment of the detectable complex for the target analyte, the first and second binding agent are selected from aptamers, antibodies, or proteins, and the second binding agent is selected from aptamers, antibodies, or proteins, in each case linked to glucose oxidase.

In a particular embodiment of the detectable complex for the target analyte, the first and second binding agent is selected from aptamers, antibodies, or proteins, and the second binding agent is selected from aptamers, antibodies, or proteins, in each case linked to glucose oxidase, galactose oxidase, D-glucose:D-fructose oxidoreductase or cellobiose oxidase.

In one embodiment of the detectable complex for the target analyte, the first and second binding agent is selected from aptamers, antibodies, or proteins, and the second binding agent are selected from aptamers, antibodies, or proteins, in each case linked to a hydrogenase enzyme.

In a particular embodiment of the detectable complex for the target analyte, the first and second binding agent is selected from aptamers, antibodies, or proteins and the second binding agent is selected from aptamers, antibodies, or proteins, in each case linked to glucose dehydrogenase, glucose 6-phosphate dehydrogenase, fructose dehydrogenase, sucrose dehydrogenase, glucoside dehydrogenase, alcohol dehydrogenase, sorbitol dehydrogenase, lactate dehydrogenase, or malate dehydrogenase.

In other embodiments, the system includes a single binding agent.

In a particular embodiment, the single binding agent is a aptamer, wherein the aptamer is bound to cellobiose oxidase. The target analyte the binds to the aptamer, which releases the cellobiose, such that oxidase is free in solution.

In a particular embodiment, the single binding agent is an aptamer, wherein the aptamer is bound to a target analyte and the target analyte is bound to an antibody-GOX complex. A signal molecule then binds and displaces the target analyte, resulting in a reduction of signal.

At present, there are no FDA approved POC devices that allow rapid self-testing at home for a subject with a test answer provided within 15, 10, 5, 1, 0.5, or 0.1 minutes, or similarly in the office, or in operational field or in resource limited environments with the low limit of detection and high accuracy required to be truly useful with respect to SARS-CoV-2 or other virus detection. POC testing can be essential for rapid detection of the infection at early stages to facilitate better disease diagnosis, monitoring and management.

In certain embodiments, the system disclosed herein is portable and suitable for use in numerous environments, including home, at work, in a clinic, emergency room, or field use and has one or more properties that are equal to or preferably, improved relative to other systems or devices for detecting target analytes, including systems or devices for detecting pathogens such as respiratory viruses and coronaviruses and more particularly, betacoronaviruses such as SARS-CoV-2. These properties may include, without limitation, speed and duration of sensing (<1 minute), specificity (>90%), selectivity (>90%), limit of detection of the assay (1 target analyte per milliliter or >100,000), quantitative detection (>90% precision and >90% accuracy, the effect of common interferents to the sensor output, cross-reactivity (>90% selectivity for target analyte) (e.g., between related viruses, such as SARS-CoV-1 and SARS-CoV-2), dynamic range, coefficient of variation of repeated measurements (<10% variance), operational stability or combinations thereof. In one embodiment, the analysis of variance with five (5) variables, depending on the statistical method used, can achieve convergence greater than 0.95 with five (5) measurements. With standardization of manufacturing, reducing the variables to one or two, the confidence level can be obtained with two (2) measurements.

In one embodiment, the system permits about 90% or greater, about 910% or greater, about 92% or greater, about 93% or greater, about 94% or greater, about 95% or greater, about 96% or greater, about 97% or greater, about 98% or greater, or about 99% or greater sensitivity.

In a particular embodiment, out of 10 tests, the system permits 9 true positive tests with 1 false negative test, wherein the true positive is a subject infected with a pathologic agent (e.g. a virus) or previously infected with the pathological agent.

In one embodiment, the system permits about 90% or greater, about 91% or greater, about 92% or greater, about 93% or greater, about 94% or greater, about 95% or greater, about 96% or greater, about 97% or greater, about 98% or greater, or about 99% or greater sensitivity.

In a particular embodiment, out of ten tests, the system permits 9 true negative tests with 1 false negative.

Other such properties may include scale of testing, assay time, ease of use and collateral (e.g., healthcare worker) infections. In particular, accuracy is of the upmost importance as a false negative result could lead an infectious individual to believe they do not have an infection (e.g., SARS-CoV-2) and, subsequently, to unknowingly infect individuals around them.

In a particular embodiment, the system disclosed herein has one or more properties that are improved relative to an RT-PCR assay performed on commercially obtained human saliva and nasal samples or samples immediately obtained from an individual. In one embodiment, the system disclosed herein as a false positive rate that is lower than an RT-PCR performed on commercially available nasal samples, and in particular, lower by about 20%, about 18%, about 16%, about 14%, about 12% about 10%, about 8%, about 6%, about 4% or about 2% or less.

In one embodiment, the system disclosed herein provides a result to the user within about 10 minutes or less from the after the addition of the biological (e.g., saliva) sample, and more particularly about 5 minutes or less, about 2 minutes, or less or about 1 minute or less. In a particular embodiment, the system permits the result to be provided to the user within about 1 to about 2 minutes.

In one embodiment, the system disclosed herein has a false positive rate of less than about 33%. In a particular embodiment, the false positive rate is about 32%, about 30%, about 28%, about 26%, about 24%, about 22%, about 20%, about 18%, about 16%, about 14%, about 12% about 10%, about 8%, about 6%, about 4%, or about 2% or less.

In another embodiment, the system disclosed herein has a false negative rate of less than about 20%, about 18%, about 16%, about 14%, about 12% about 10%, about 8%, about 6%, about 4%, or about 2% or less.

In one embodiment, the system disclosed herein permits detection within the range of about 10¹ to about 10¹¹ viral copies per mL. In one embodiment, the system disclosed herein permits detection of at least 10¹, at least 10², at least 10³, at least 10⁴, at least 10⁵, at least 10⁶, at least 10⁷, at least 10⁸, at least 10⁹, at least 10¹⁰, or at least 10¹¹ or more viral copies per mL.

In one embodiment, the system disclosed herein permits detection of about 10 to 1,000 viruses in solution, well below the current clinical range of interest. In one embodiment, the system permits detection of about 10 to about 100 viruses in solution, or about 10 to about 50, and more particularly about 10 to about 20 viruses in solution.

In one embodiment, the system disclosed herein with a limit of detection of about 10 viral copies/mL or 10 analyte per mL or similar concentration.

In one embodiment, the systems disclosed herein permits (e.g., with a 95% confidence interval) a 95% sensitivity and 95% specificity.

In another embodiment, the system disclosed herein permits a minimal target clinical sensitivity of about 90%, and optimal target sensitivity of about 98%. In another embodiment, the system disclosed herein permits a minimal target specificity of about 90%, and an optimal target is >98%.

In one embodiment, the system disclosed herein permits improved disease diagnosis, monitoring, management or combinations thereof.

In certain embodiments, the system stores multiple test results for the same user taken at different times and comparing these to monitor or predict the likely development of a disease or condition (e.g., COVID-19). In one embodiment, the system permits obtaining two or more results, three or more results, or five or more results with respect to the quantity of a target analyte for the same user at different times, to permit monitoring of a trend in analyte level over time.

In certain embodiments, the system disclosed herein permits selection of a treatment modality for prevention or treatment of a disease (e.g., COVID-19). The treatment modality may differ and include, for example, a small molecule therapeutic agent, a biologic agent (e.g., a protein, antibody, therapeutic vaccine).

In certain embodiments, the system disclosed herein permits monitoring the effectiveness of one more therapeutic agents (e.g., anti-viral agents) and permits the user to seek an alternative therapeutic approach if the therapeutic agent is not sufficiently effective over a period of time. In certain embodiments, the system stores multiple test results for the same user taken at different times and permits comparing these test results to monitor a treatment regime associated with variation in the levels of a given analyte. In one embodiment, if the treatment regime does not produce a reduction in the level of an analyte (e.g., viral count) within a defined period (e.g., days), the user may discontinue the treatment regime in favor of an alternative treatment regime or in certain embodiments, supplement the treatment regime with a second treatment regime. In one embodiment, the system permits obtaining two or more results, three or more results, or five or more results with respect to the quantity of a target analyte for the same user at different times, to permit monitoring of a trend in analyte level over time.

In certain embodiments, the system disclosed herein advantageously permits one or more of the following: (i) detection of viral antigen in saliva (i.e., eliminating the use of uncomfortable sample collection techniques); (ii) straightforward saliva sample collection; (iii) use of existing glucometer technology that is widely available and relatively inexpensive; or (iv) a test strip adaptable to the detection of other pathological agents (e.g., viruses).

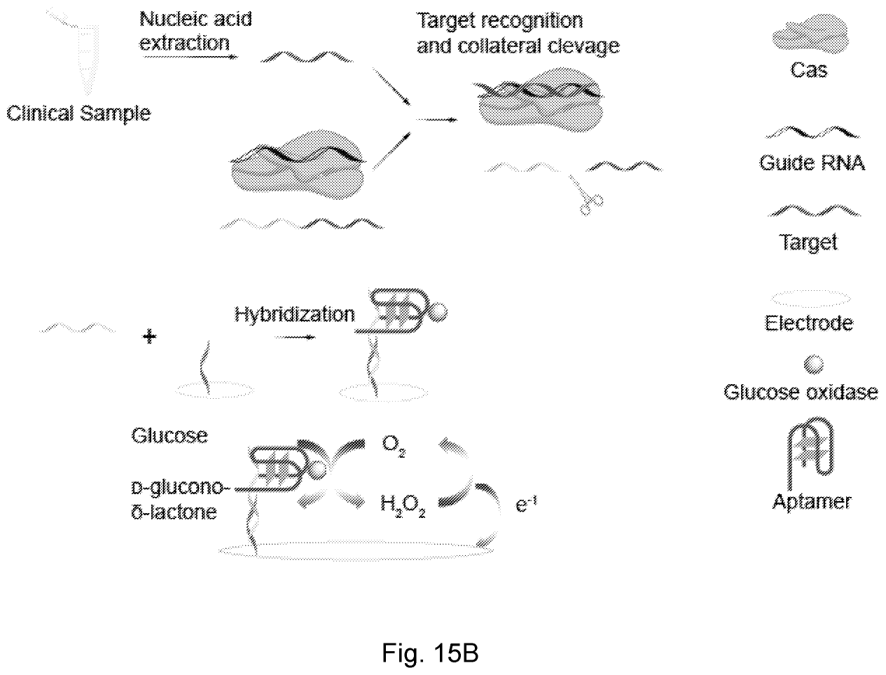

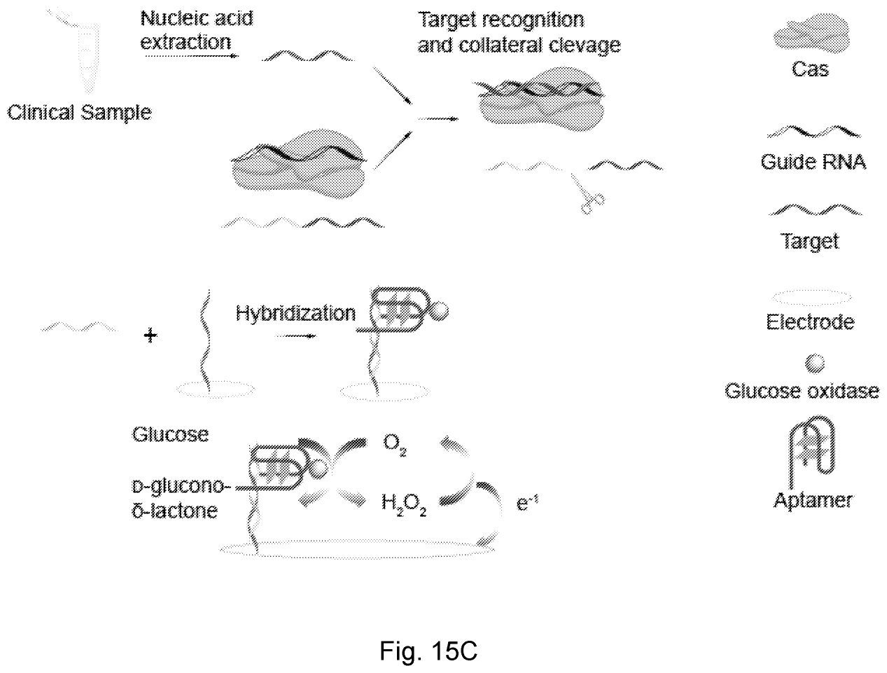

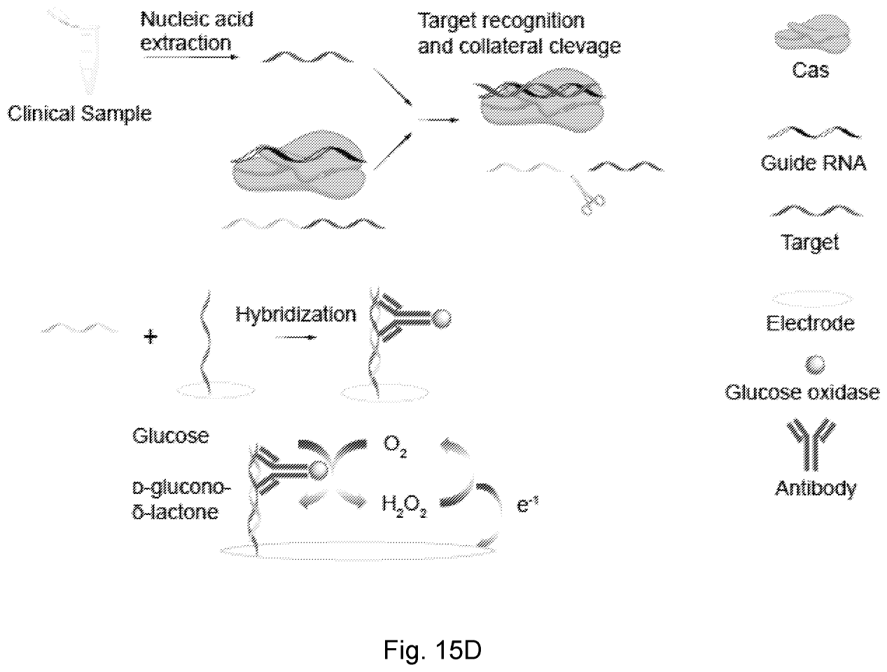

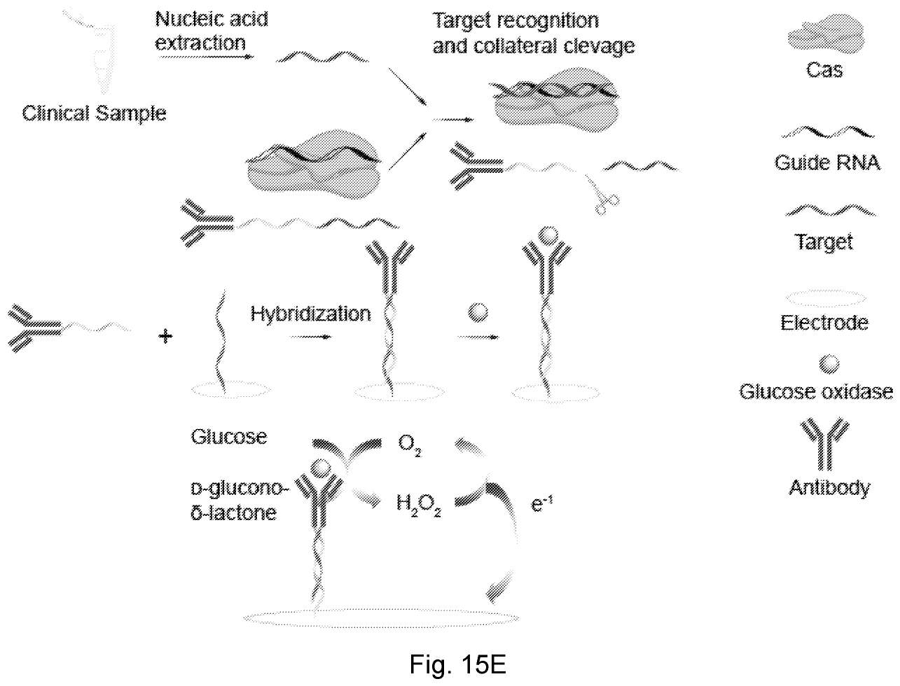

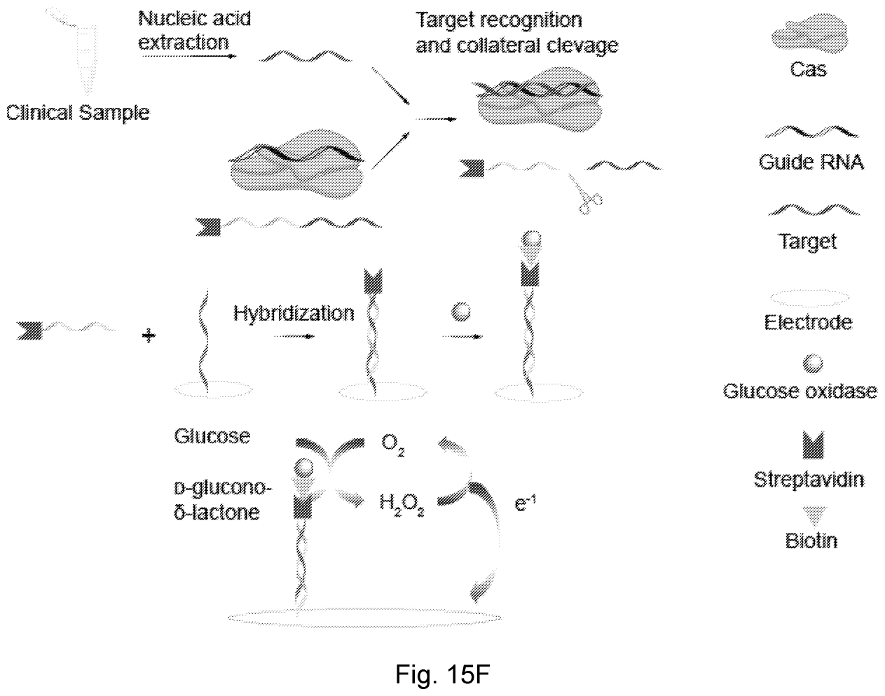

In another aspect, described herein is a system (referred to herein as a nucleic acid detection system; see e.g., FIG. 15A-15F) for detecting at least one target nucleic acid in a biological sample, wherein the system comprises (i) a sequence-specific endonuclease and guide nucleic acid that cleave a collateral nucleic acid upon specific binding of the target nucleic acid to the endonuclease and guide nucleic acid; (ii) a detection nucleic acid that is capable of creating a detectable complex with the cleaved collateral nucleic acid; and (iii) a detection device for detecting the detectable complex, wherein the detection device is an oxidase-based amperometric sensor and the biological sample is present in sweat, salvia, serum, mucus, or blood.

The one or more targets detected or monitor utilizing the systems and methods herein may be present in a biological sample (e.g., a liquid biological sample) collected from a subject, e.g., a human subject.

The biological sample may vary and include, for example, of blood, serum, milk, sweat, semen, ejaculate, mucus, tears, saliva, plasma, secretions of the genito-urinary tract, lymph fluid, urine, white blood cells, pleural fluid, ascites, sputum, peritoneal fluid, cerebrospinal fluid, pleural fluid, pericardial fluid, amniotic fluid, synovial fluid, interstitial fluid, fecal matter, sebum, respiratory droplets, semen, vaginal mucus, cerumen, epidermal cells, and any combinations or mixtures of the aforementioned items.

In one embodiment, the biological sample is not blood. In a particular embodiment, the biological sample is saliva. Saliva is a viscous, dense, sticky fluid innately containing microorganisms like bacteria and fungi, intact human cells, cellular debris, and many soluble materials enzymes, hormones, antibodies, and other molecules.

Saliva specimens can be readily collected from a subject in any suitable manner and in certain embodiments, without the use of specialized equipment, e.g., by having the subject split into a vessel, the contents of which are then diluted and applied to the test strip or alternatively, spit on the test strip directly. See, e.g., Navazesn M (1993). Methods for collecting saliva. Ann N Y Acad Sci 694:72-77.

The volume of the biological sample may vary. In one embodiment, the volume of the biological sample is between about 1 μL, 10 μL, 20 μL, 50 μL, or 100 μL and about 2000 μL, more particularly about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, about 1000 μL, about 1250 μL, about 1500 μL, about 1750 μL, or about 2000 μL.

In certain embodiments, the biological sample is pre-processed prior to use in the systems and methods disclosed herein. For example, the saliva can be processed (e.g., by centrifugation) to provide a cell-free fluid phase.

The systems and methods disclosed herein are designed to detect one or more targets.

Representative non-limiting targets include target including virus pathogens.

In some embodiments, the one or more target molecules is a viral antigen. In the context of the invention the term “viral antigen” is to be understood as a protein, subunit, or fragment thereof encoded by the viral genome, or a nucleic acid associated with the virus (e.g., viral genome or viral transcript).

The virus may vary and includes, without limitation, respiratory viruses and coronaviruses.

In one embodiment, the target molecule is a viral antigen associated with a coronavirus. Coronaviruses consist of a large and diverse family of enveloped, positive-sense, single-stranded RNA viruses. Every coronavirus contains four structural proteins, for example spike (S), envelope (E), membrane (M), and nucleocapsid (N) proteins. Among them, S protein plays the most important roles in viral attachment, fusion, and entry.

The S protein is a trimeric type-I transmembrane glycoprotein, which forms the characteristic corona of large protruding spikes on the virion surface and mediate binding to host cell receptors and fusion with the host cell membrane. In many coronaviruses, S is post-translationally cleaved into two subunits, designated S1 and S2, which trimerize and fold into a metastable pre-fusion conformation. The S1 subunit forms the “head” of the spike and contains two domains: an amino (N)-terminal domain (NTD) and a carboxy (C)-terminal domain (CTD), with the latter generally containing a receptor binding domain (RBD). The S2 subunit contains two heptad repeat (HR) regions. When S1 recognizes and binds to the corresponding host receptor, S2 undergoes a conformation change, extending itself from compressed form to a nail-like shape, termed as post-fusion state. This permits the viral envelope to fuse with the outer membrane and deposit the viral genetic material inside the cell. The life cycle of the virus then progresses to include biosynthesis, assembly and release.

In one embodiment, the target molecule is S1 or S2 and more particularly, the NTD, the RBD, CTD1, CTD2, S1/S2, S1/S2 cleavage site, S2′, S2′ cleavage site, fusion peptide, fusion peptide proximal region (FPPR), heptad repeat 1 (HR1), heptad repeat 1, central helix region (CHD), connector domain (CD, heptad repeat 2 (heptad repeat 2), transmembrane anchor (TM), or cytoplasmic tail (CT), or a combination thereof.

Coronavirus diversity is reflected in the variable S proteins, which have evolved into forms differing in their receptor interactions and their response to various environmental triggers of virus—cell membrane fusion. In particular, the RBD of the S protein is the most variable genomic part in the betacoronavirus group.

Four serologically distinct groups of coronaviruses have been described, i.e., alpha, beta (previously referred to as group 2), delta, and gamma. Within each group, viruses are characterized by their host range and genome sequence. The alphacoronaviruses and betacoronaviruses infect only mammals, while the gammacoronaviruses and deltacoronaviruses primarily infect birds, although some of them can also infect mammals. Novel mammalian coronaviruses are now regularly identified. (see e.g., Su et al., Trends Microbiol. 2016; 24: 490-502). Betacoronaviruses (Beta-CoV) of known clinical important to humans includes viruses of the A, B and C lineage and more particularly, the A lineage: OC43 (which can cause the common cold) and HKU1; the B lineage: SARS-CoV and SARS-CoV-2 (which causes the disease COVID-19); and the C lineage: MERS-CoV.

In one embodiment, the systems disclosed herein are directed to the detection of a betacoronavirus infection and more particularly, an A-lineage, B-lineage, or C lineage coronavirus infection. These are viruses with a positive-sense single-strand RNA of around 32 Kb, encoding for multiple structural and non-structural proteins. The viral particles contain four main structural proteins: the spike, membrane, envelope protein, and nucleocapsid. The spike protein protrudes from the envelope of the virion and plays a pivotal role in the receptor host selectivity and cellular attachment. Betacoronaviruses have many similarities within the ORFlab polyprotein and most structural proteins; however, the spike protein and accessory proteins portray significant diversity. Mutations in the spike protein could change the tropism of a virus, including new hosts or increasing pathogenesis

In another particular embodiment, the systems and methods disclosed herein are directed to the detection of a severe acute respiratory syndrome coronavirus 2 (SARS-CoV-2) infections. SARS-CoV-2 (also referred to as 2019-nCoV) was identified in January 2020 as the causative agent of Severe Acute Respiratory Syndrome 2, also referred to as Covid-19. Infections with the novel coronavirus quickly became widespread and in March 2020, the World Health Organization (WHO) declared Covid-19 a pandemic. The virus has, as of June 2021, infected more than 178 million people and killed more than 3.85 million individuals. Individual living or working in high density and close contact (e.g., military personnel) are particularly at risk.

Clinical signs associated with SARS-CoV-2 include pneumonia, fever, dry cough, headache, and dyspnea, which may progress to respiratory failure and death. The incubation period for SARS-CoV-2 of 2 to 14 days can be longer than for SARS-CoV and MERS-CoV, which have a mean incubation time of 5 to 7 days.

SARS-CoV-2 was sequenced and isolated by January 2020 (see e.g., e.g., Zhou N. N Engl J Med., 382 (2020), pp. 727-733). Several sequences of SARS-CoV-2 have since been released. In some embodiments of any of the aspects, the target analyte (e.g., protein, glycoprotein, or nucleic acid) comprises at least a portion of Severe acute respiratory syndrome coronavirus 2 isolate SARS-CoV-2, (see e.g., complete genome, SARS-CoV-2 Jan. 2020/NC_045512.2 Assembly (wuhCor1)).

In some embodiments of any of the aspects, the target analyte comprises SEQ ID NO: 1 or SEQ ID NO: 2 (Severe acute respiratory syndrome coronavirus 2 isolate SARS-CoV-2, S gene). In some embodiments of any of the aspects, the target nucleic acid comprises SEQ ID NO: 1 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1 that maintains the same function or a codon-optimized version of SEQ ID NO: 1. In some embodiments of any of the aspects, the target nucleic acid comprises SEQ ID NO: 1 or a nucleic acid sequence that is at least 95% identical to SEQ ID NO: 1 that maintains the same function or a codon-optimized version of SEQ ID NO: 1.

In some embodiments of any of the aspects, the target polypeptide comprises SEQ ID NO: 2 or SEQ ID NO: 3 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 2-3 that maintains the same function. In some embodiments of any of the aspects, the target polypeptide comprises one of SEQ ID NOs: 2-3 or an amino acid sequence that is at least 95% identical to SEQ ID NOs: 2-3 that maintains the same function.

SEQ ID NO: 1, Severe acute respiratory syndrome coronavirus 2 isolate Wuhan-Hu-1, S surface glycoprotein, Gene ID: 43740568, 3822 bp ss-RNA, NC_045512 REGION: 21563-25384

atgtttgtttttcttgttttattgccactagtctctagtc
agtgtgttaatcttacaaccagaactcaattaccccctgc
atacactaattctttcacacgtggtgtttattaccctgac
aaagttttcagatcctcagttttacattcaactcaggact
tgttcttacctttcttttccaatgttacttggttccatgc
tatacatgtctctgggaccaatggtactaagaggtttgat
aaccctgtcctaccatttaatgatggtgtttattttgctt
ccactgagaagtctaacataataagaggctggatttttgg
tactactttagattcgaagacccagtccctacttattgtt
aataacgctactaatgttgttattaaagtctgtgaatttc
aattttgtaatgatccatttttgggtgtttattaccacaa
aaacaacaaaagttggatggaaagtgagttcagagtttat
tctagtgcgaataattgcacttttgaatatgtctctcagc
cttttcttatggaccttgaaggaaaacagggtaatttcaa
aaatcttagggaatttgtgtttaagaatattgatggttat
tttaaaatatattctaagcacacgcctattaatttagtgc
gtgatctccctcagggtttttcggctttagaaccattggt
agatttgccaataggtattaacatcactaggtttcaaact
ttacttgctttacatagaagttatttgactcctggtgatt
cttcttcaggttggacagctggtgctgcagcttattatgt
gggttatcttcaacctaggacttttctattaaaatataat
gaaaatggaaccattacagatgctgtagactgtgcacttg
accctctctcagaaacaaagtgtacgttgaaatccttcac
tgtagaaaaaggaatctatcaaacttctaactttagagtc
caaccaacagaatctattgttagatttcctaatattacaa
acttgtgcccttttggtgaagtttttaacgccaccagatt
tgcatctgtttatgcttggaacaggaagagaatcagcaac
tgtgttgctgattattctgtcctatataattccgcatcat
tttccacttttaagtgttatggagtgtctcctactaaatt
aaatgatctctgctttactaatgtctatgcagattcattt
gtaattagaggtgatgaagtcagacaaatcgctccagggc
aaactggaaagattgctgattataattataaattaccaga
tgattttacaggctgcgttatagcttggaattctaacaat
cttgattctaaggttggtggtaattataattacctgtata
gattgtttaggaagtctaatctcaaaccttttgagagaga
tatttcaactgaaatctatcaggccggtagcacaccttgt
aatggtgttgaaggttttaattgttactttcctttacaat
catatggtttccaacccactaatggtgttggttaccaacc
atacagagtagtagtactttcttttgaacttctacatgca
ccagcaactgtttgtggacctaaaaagtctactaatttgg
ttaaaaacaaatgtgtcaatttcaacttcaatggtttaac
aggcacaggtgttcttactgagtctaacaaaaagtttctg
cctttccaacaatttggcagagacattgctgacactactg
atgctgtccgtgatccacagacacttgagattcttgacat
tacaccatgttcttttggtggtgtcagtgttataacacca
ggaacaaatacttctaaccaggttgctgttctttatcagg
atgttaactgcacagaagtccctgttgctattcatgcaga
tcaacttactcctacttggcgtgtttattctacaggttct
aatgtttttcaaacacgtgcaggctgtttaataggggctg
aacatgtcaacaactcatatgagtgtgacatacccattgg
tgcaggtatatgcgctagttatcagactcagactaattct
cctcggcgggcacgtagtgtagctagtcaatccatcattg
cctacactatgtcacttggtgcagaaaattcagttgctta
ctctaataactctattgccatacccacaaattttactatt
agtgttaccacagaaattctaccagtgtctatgaccaaga
catcagtagattgtacaatgtacatttgtggtgattcaac
tgaatgcagcaatcttttgttgcaatatggcagtttttgt
acacaattaaaccgtgctttaactggaatagctgttgaac
aagacaaaaacacccaagaagtttttgcacaagtcaaaca
aatttacaaaacaccaccaattaaagattttggtggtttt
aatttttcacaaatattaccagatccatcaaaaccaagca
agaggtcatttattgaagatctacttttcaacaaagtgac
acttgcagatgctggcttcatcaaacaatatggtgattgc
cttggtgatattgctgctagagacctcatttgtgcacaaa
agtttaacggccttactgttttgccacctttgctcacaga
tgaaatgattgctcaatacacttctgcactgttagcgggt
acaatcacttctggttggacctttggtgcaggtgctgcat
tacaaataccatttgctatgcaaatggcttataggtttaa
tggtattggagttacacagaatgttctctatgagaaccaa
aaattgattgccaaccaatttaatagtgctattggcaaaa
ttcaagactcactttcttccacagcaagtgcacttggaaa
acttcaagatgtggtcaaccaaaatgcacaagctttaaac
acgcttgttaaacaacttagctccaattttggtgcaattt
caagtgttttaaatgatatcctttcacgtcttgacaaagt
tgaggctgaagtgcaaattgataggttgatcacaggcaga
cttcaaagtttgcagacatatgtgactcaacaattaatta
gagctgcagaaatcagagcttctgctaatcttgctgctac
taaaatgtcagagtgtgtacttggacaatcaaaaagagtt
gatttttgtggaaagggctatcatcttatgtccttccctc
agtcagcacctcatggtgtagtcttcttgcatgtgactta
tgtccctgcacaagaaaagaacttcacaactgctcctgcc
atttgtcatgatggaaaagcacactttcctcgtgaaggtg
tctttgtttcaaatggcacacactggtttgtaacacaaag
gaatttttatgaaccacaaatcattactacagacaacaca
tttgtgtctggtaactgtgatgttgtaataggaattgtca
acaacacagtttatgatcctttgcaacctgaattagactc
attcaaggaggagttagataaatattttaagaatcataca
tcaccagatgttgatttaggtgacatctctggcattaatg
cttcagttgtaaacattcaaaaagaaattgaccgcctcaa
tgaggttgccaagaatttaaatgaatctctcatcgatctc
caagaacttggaaagtatgagcagtatataaaatggccat
ggtacatttggctaggttttatagctggcttgattgccat
agtaatggtgacaattatgctttgctgtatgaccagttgc
tgtagttgtctcaagggctgttgttcttgtggatcctgct
gcaaatttgatgaagacgactctgagccagtgctcaaagg
agtcaaattacattacacataa

SEQ ID NO: 2, Severe acute respiratory syndrome coronavirus 2 isolate Wuhan-Hu-1, S surface glycoprotein, Gene ID: 43740568, 1273 aa

MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTECSNLLLQYGSFC
TQLNRALTGIAVEQDKNTQEVFAQVKQIYKTPPIKDFGGF
NFSQILPDPSKPSKRSFIEDLLFNKVTLADAGFIKQYGDC
LGDIAARDLICAQKFNGLTVLPPLLTDEMIAQYTSALLAG
TITSGWTFGAGAALQIPFAMQMAYRFNGIGVTQNVLYENQ
KLIANQFNSAIGKIQDSLSSTASALGKLQDVVNQNAQALN
TLVKQLSSNFGAISSVLNDILSRLDKVEAEVQIDRLITGR
LQSLQTYVTQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHT
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYT

SEQ ID NO: 3, SARS-CoV-2, receptor binding domain (RBD) of the spike protein, 194 aa (see e.g., FIG. 9); corresponds to amino acids (aa) 331-524 of SEQ ID NO: 2

NITNLCPFGEVFNATRFASVYAWNRKRISNCVADYSVLYN
SASFSTFKCYGVSPTKLNDLCFTNVYADSFVIRGDEVRQI
APGQTGKIADYNYKLPDDFTGCVIAWNSNNLDSKVGGNYN
YLYRLFRKSNLKPFERDISTEIYQAGSTPCNGVEGFNCYF
PLQSYGFQPTNGVGYQPYRVVVLSFELLHAPATV

Similar to other coronaviruses, the spike (S) protein is the major glycoprotein on the SARS-CoV-2 virus surface. SARS-CoV-2 seems to have a receptor binding domain (RBD that binds with high affinity to ACE2 from humans, ferrets, cats and other species with high receptor homology. (Wan et al., (2020) J. Virol. doi.org/10.1128/JVI.00127-20).

In some embodiments, the SARS-CoV-2 S1 RBD is 194 amino acids in length (e.g., N331-V524 of SEQ ID NO: 2; see e.g., SEQ ID NO: 3). In some embodiments, the SARS-CoV-2 S1 RBD is the corresponding region to the RBD of SARS-CoV S, which is 193 amino acids in length (e.g., N318-V510 of SEQ ID NO: 5; see e.g., SEQ ID NO: 6).

It has been reported that the SARS-CoV-2 S protein shares 76% amino acid sequence identity with the SARS-CoV S Urbani and 80% identity with bat SARSr-CoV ZXC21 S and ZC45 S glycoprotein. FIG. 9 shows receptor binding domain (RBD) of the spike protein sequence alignment of SARS-CoV-2 and other related coronaviruses. Sequence alignments are available for the interacting domain of SARS-CoV-2 (see e.g., NCBI accession number MN938384), Bat-CoV (see e.g., NCBI accession numbers MN996532 or MG772933) and SARS-CoV (see e.g., NCBI accession number NC004718). The RBD of SARS-CoV-2 differs largely from the SARS-CoV at the C-terminus residues.

The S1 subunit of SARS-CoV-2 contains a receptor-binding domain (RBD), while the S2 subunit contains a hydrophobic fusion peptide and two heptad repeat regions. S1 contains two structurally independent domains, the N-terminal domain (NTD) and the C-terminal domain (C-domain). Depending on the virus, either the NTD or the C-domain can serve as the receptor-binding domain (RBD).

In one embodiment, the systems and methods disclosed herein permit detection of the S protein of SARS-CoV-2 or a subunit or fragment thereof, and more particularly, one or more epitopes of the S protein of SARS-CoV-2, including, but not limited to the RBD, the S1 amino-terminal domain (S1-NTD), ORF3 (3a and 3b) and the accessory gene ORF8.

In one embodiment, the systems and methods herein permit detection of whole virus, i.e., a SARS-CoV-2 particle.

In one embodiment, the systems and methods herein permit detection of one or more epitopes of the N-terminal domain (NTD) and the C-terminal domain (C-domain) of SARS-CoV-2.

In one embodiment, the systems and methods disclosed herein permit detection of the S protein of SARS-CoV-2 or a subunit or fragment thereof, and more particularly, one or more epitopes of the S protein of SARS-CoV-2, including, but not limited to the RBD.

In one embodiment, the systems and methods herein permit detection of one or more epitopes in the RBD of SARS-CoV-2 and more particularly, one or more epitopes residues within residues 331 and 524 of the RBD (see e.g., SEQ ID NO: 3).

In one embodiment, the systems and methods herein permit detection of one or more epitopes in the RBD of SARS-CoV-2 and more particularly, one or more epitopes residues within residues 318 and 510 of the RBD.

In one embodiment, the systems and methods herein permit detection of one or more epitopes in the RBD of SARS-CoV-2 and more particularly, one or more epitopes residues within residues 319 and 510 of the RBD.

In a particular embodiment, the systems and methods disclosed herein are directed to the detection of a SARS-CoV infection. SARS-CoV was identified in April 2003 as the pathogen responsible for Severe Acute Respiratory Syndrome (SARS) (see e.g., Drosten et al., New Engl. J. Med. 2003; 348: 1967-1976). Clinically, SARS-CoV exhibits biphasic course, i.e., first high fever, parainfluenza syndrome followed by increasing respiratory distress. Droplets play a key role in transmission. Diagnosis is based on clinical picture and epidemiological data supported by positive serology, PCR or presence virus in cell culture. The consensus genomic sequence for SARS-CoV was published shortly thereafter, resembling most closely the group B betacoronaviruses (see e.g., Marra et al., Science. 2003; 300: 1399-14040; Ruan et al., Lancet. 2003; 361: 1779-1785).

The SARS-CoV spike protein has been shown to consist of two functional domains, S1 (amino acids 12-680) and S2 (amino acids 681-1255) (see e.g., Li et al., Science. 2005; 309: 1864-1868). The RBD is located within the S1 subunit and has been mapped to a fragment consisting of amino acids (aa) 318-510 in the S1 domain. (see e.g., Wong et al., J Biol Chem. 2004; 279: 3197-3201).

In one embodiment, the systems and methods disclosed herein permit detection of the S protein of SARS-CoV or a subunit or fragment thereof, and more particularly, one or more epitopes of the S protein of SARS-CoV, including, but not limited to the RBD.

In one embodiment, the systems and methods herein permit detection of one or more epitopes in the RBD of SARS-CoV and more particularly, one or more epitopes residues within residues 318 and 510 of the RBD.

In some embodiments of any of the aspects, the target analyte (e.g., protein, glycoprotein, or nucleic acid) comprises at least a portion of SARS-CoV, (see e.g., complete genome, NCBI Reference Sequence: NC_004718).

In some embodiments of any of the aspects, the target analyte comprises one of SEQ ID NOs: 4-6 (SARS-CoV S gene). In some embodiments of any of the aspects, the target nucleic acid comprises SEQ ID NO: 4 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 4 that maintains the same function or a codon-optimized version of SEQ ID NO: 4. In some embodiments of any of the aspects, the target nucleic acid comprises SEQ ID NO: 4 or a nucleic acid sequence that is at least 95% identical to SEQ ID NO: 4 that maintains the same function or a codon-optimized version of SEQ ID NO: 4.

In some embodiments of any of the aspects, the target polypeptide comprises SEQ ID NO: 5 or SEQ ID NO: 6 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 5-6 that maintains the same function. In some embodiments of any of the aspects, the target polypeptide comprises one of SEQ ID NO: 5-6 or an amino acid sequence that is at least 95% identical to one of SEQ ID NO: 5-6 that maintains the same function.

SEQ ID NO: 4, spike glycoprotein SARS coronavirus Tor2, NCBI Reference Sequence: NC_004718.3 REGION: 21492-25259, 3768 bp

atgtttattttcttattatttcttactctcactagtggta
gtgaccttgaccggtgcaccacttttgatgatgttcaagc
tcctaattacactcaacatacttcatctatgaggggggtt
tactatcctgatgaaatttttagatcagacactctttatt
taactcaggatttatttcttccattttattctaatgttac
agggtttcatactattaatcatacgtttggcaaccctgtc
ataccttttaaggatggtatttattttgctgccacagaga
aatcaaatgttgtccgtggttgggtttttggttctaccat
gaacaacaagtcacagtcggtgattattattaacaattct
actaatgttgttatacgagcatgtaactttgaattgtgtg
acaaccctttctttgctgtttctaaacccatgggtacaca
gacacatactatgatattcgataatgcatttaattgcact
ttcgagtacatatctgatgccttttcgcttgatgtttcag
aaaagtcaggtaattttaaacacttacgagagtttgtgtt
taaaaataaagatgggtttctctatgtttataagggctat
caacctatagatgtagttcgtgatctaccttctggtttta
acactttgaaacctatttttaagttgcctcttggtattaa
cattacaaattttagagccattcttacagccttttcacct
gctcaagacatttggggcacgtcagctgcagcctattttg
ttggctatttaaagccaactacatttatgctcaagtatga
tgaaaatggtacaatcacagatgctgttgattgttctcaa
aatccacttgctgaactcaaatgctctgttaagagctttg
agattgacaaaggaatttaccagacctctaatttcagggt
tgttccctcaggagatgttgtgagattccctaatattaca
aacttgtgtccttttggagaggtttttaatgctactaaat
tcccttctgtctatgcatgggagagaaaaaaaatttctaa
ttgtgttgctgattactctgtgctctacaactcaacattt
ttttcaacctttaagtgctatggcgtttctgccactaagt
tgaatgatctttgcttctccaatgtctatgcagattcttt
tgtagtcaagggagatgatgtaagacaaatagcgccagga
caaactggtgttattgctgattataattataaattgccag
atgatttcatgggttgtgtccttgcttggaatactaggaa
cattgatgctacttcaactggtaattataattataaatat
aggtatcttagacatggcaagcttaggccctttgagagag
acatatctaatgtgcctttctcccctgatggcaaaccttg
caccccacctgctcttaattgttattggccattaaatgat
tatggtttttacaccactactggcattggctaccaacctt
acagagttgtagtactttcttttgaacttttaaatgcacc
ggccacggtttgtggaccaaaattatccactgaccttatt
aagaaccagtgtgtcaattttaattttaatggactcactg
gtactggtgtgttaactccttcttcaaagagatttcaacc
atttcaacaatttggccgtgatgtttctgatttcactgat
tccgttcgagatcctaaaacatctgaaatattagacattt
caccttgcgcttttgggggtgtaagtgtaattacacctgg
aacaaatgcttcatctgaagttgctgttctatatcaagat
gttaactgcactgatgtttctacagcaattcatgcagatc
aactcacaccagcttggcgcatatattctactggaaacaa
tgtattccagactcaagcaggctgtcttataggagctgag
catgtcgacacttcttatgagtgcgacattcctattggag
ctggcatttgtgctagttaccatacagtttctttattacg
tagtactagccaaaaatctattgtggcttatactatgtct
ttaggtgctgatagttcaattgcttactctaataacacca
ttgctatacctactaacttttcaattagcattactacaga
agtaatgcctgtttctatggctaaaacctccgtagattgt
aatatgtacatctgcggagattctactgaatgtgctaatt
tgcttctccaatatggtagcttttgcacacaactaaatcg
tgcactctcaggtattgctgctgaacaggatcgcaacaca
cgtgaagtgttcgctcaagtcaaacaaatgtacaaaaccc
caactttgaaatattttggtggttttaatttttcacaaat
attacctgaccctctaaagccaactaagaggtcttttatt
gaggacttgctctttaataaggtgacactcgctgatgctg
gcttcatgaagcaatatggcgaatgcctaggtgatattaa
tgctagagatctcatttgtgcgcagaagttcaatggactt
acagtgttgccacctctgctcactgatgatatgattgctg
cctacactgctgctctagttagtggtactgccactgctgg
atggacatttggtgctggcgctgctcttcaaatacctttt
gctatgcaaatggcatataggttcaatggcattggagtta
cccaaaatgttctctatgagaaccaaaaacaaatcgccaa
ccaatttaacaaggcgattagtcaaattcaagaatcactt
acaacaacatcaactgcattgggcaagctgcaagacgttg
ttaaccagaatgctcaagcattaaacacacttgttaaaca
acttagctctaattttggtgcaatttcaagtgtgctaaat
gatatcctttcgcgacttgataaagtcgaggcggaggtac
aaattgacaggttaattacaggcagacttcaaagccttca
aacctatgtaacacaacaactaatcagggctgctgaaatc
agggcttctgctaatcttgctgctactaaaatgtctgagt
gtgttcttggacaatcaaaaagagttgacttttgtggaaa
gggctaccaccttatgtccttcccacaagcagccccgcat
ggtgttgtcttcctacatgtcacgtatgtgccatcccagg
agaggaacttcaccacagcgccagcaatttgtcatgaagg
caaagcatacttccctcgtgaaggtgtttttgtgtttaat
ggcacttcttggtttattacacagaggaacttcttttctc
cacaaataattactacagacaatacatttgtctcaggaaa
ttgtgatgtcgttattggcatcattaacaacacagtttat
gatcctctgcaacctgagcttgactcattcaaagaagagc
tggacaagtacttcaaaaatcatacatcaccagatgttga
tcttggcgacatttcaggcattaacgcttctgtcgtcaac
attcaaaaagaaattgaccgcctcaatgaggtcgctaaaa
atttaaatgaatcactcattgaccttcaagaattgggaaa
atatgagcaatatattaaatggccttggtatgtttggctc
ggcttcattgctggactaattgccatcgtcatggttacaa
tcttgctttgttgcatgactagttgttgcagttgcctcaa
gggtgcatgctcttgtggttcttgctgcaagtttgatgag
gatgactctgagccagttctcaagggtgtcaaattacatt
acacataa

SEQ ID NO: 5, spike glycoprotein SARS coronavirus Tor2, NCBI Reference Sequence: YP_009825051.1, 1255 aa

MFIFLLFLTLTSGSDLDRCTTFDDVQAPNYTQHTSSMRGV
YYPDEIFRSDTLYLTQDLFLPFYSNVTGFHTINHTFGNPV
IPFKDGIYFAATEKSNVVRGWVFGSTMNNKSQSVIIINNS
TNVVIRACNFELCDNPFFAVSKPMGTQTHTMIFDNAFNCT
FEYISDAFSLDVSEKSGNFKHLREFVFKNKDGFLYVYKGY
QPIDVVRDLPSGFNTLKPIFKLPLGINITNFRAILTAFSP
AQDIWGTSAAAYFVGYLKPTTFMLKYDENGTITDAVDCSQ
NPLAELKCSVKSFEIDKGIYQTSNFRVVPSGDVVRFPNIT
NLCPFGEVFNATKFPSVYAWERKKISNCVADYSVLYNSTF
FSTFKCYGVSATKLNDLCFSNVYADSFVVKGDDVRQIAPG
QTGVIADYNYKLPDDFMGCVLAWNTRNIDATSTGNYNYKY
RYLRHGKLRPFERDISNVPFSPDGKPCTPPALNCYWPLND
YGFYTTTGIGYQPYRVVVLSFELLNAPATVCGPKLSTDLI
KNQCVNFNFNGLTGTGVLTPSSKRFQPFQQFGRDVSDFTD
SVRDPKTSEILDISPCAFGGVSVITPGTNASSEVAVLYQD
VNCTDVSTAIHADQLTPAWRIYSTGNNVFQTQAGCLIGAE
HVDTSYECDIPIGAGICASYHTVSLLRSTSQKSIVAYTMS
LGADSSIAYSNNTIAIPTNFSISITTEVMPVSMAKTSVDC
NMYICGDSTECANLLLQYGSFCTQLNRALSGIAAEQDRNT
REVFAQVKQMYKTPTLKYFGGFNFSQILPDPLKPTKRSFI
EDLLFNKVTLADAGFMKQYGECLGDINARDLICAQKFNGL
TVLPPLLTDDMIAAYTAALVSGTATAGWTFGAGAALQIPF
AMQMAYRFNGIGVTQNVLYENQKQIANQFNKAISQIQESL
TTTSTALGKLQDVVNQNAQALNTLVKQLSSNFGAISSVLN
DILSRLDKVEAEVQIDRLITGRLQSLQTYVTQQLIRAAEI
RASANLAATKMSECVLGQSKRVDFCGKGYHLMSFPQAAPH
GVVFLHVTYVPSQERNFTTAPAICHEGKAYFPREGVFVFN
GTSWFITQRNFFSPQIITTDNTFVSGNCDVVIGIINNTVY
DPLQPELDSFKEELDKYFKNHTSPDVDLGDISGINASVVN
IQKEIDRLNEVAKNLNESLIDLQELGKYEQYIKWPWYVWL
GFIAGLIAIVMVTILLCCMTSCCSCLKGACSCGSCCKFDE
DDSEPVLKGVKLHYT

SEQ ID NO: 6, SARS-CoV, receptor binding domain (RBD) of the spike protein, 193 aa (see e.g., FIG. 9); corresponds to amino acids (aa) 318-510 of SEQ ID NO: 5

NITNLCPFGEVFNATKFPSVYAWERKKISNCVADYSVLYN
STFFSTFKCYGVSATKLNDLCFSNVYADSFVVKGDDVRQI
APGQTGVIADYNYKLPDDFMGCVLAWNTRNIDATSTGNYN
YKYRYLRHGKLRPFERDISNVPFSPDGKPCTPPALNCYWP
LNDYGFYTTTGIGYQPYRVVVLSFELLNAPATV

In a particular embodiment, the systems and methods disclosed herein are directed to the detection of a Middle East Respiratory Syndrome-Coronavirus (MERS-CoV) infectious. MERS-CoV is a newly-emergent betacoronavirus which causes severe acute respiratory disease. It was first isolated in Saudi Arabia in 2012 (see e.g., Zaki et al 2012, NEJM 367: 1814-1820) and since then has spread to about 18 countries with most of the cases in Saudi Arabia and United Arab Emirates. Clinical features of MERS-CoV infection in humans range from an asymptomatic infection to very severe pneumonia, with potential development of acute respiratory distress syndrome, septic shock, and multi-organ failure resulting in death. The virus uses its spike protein for interaction with a cellular receptor for entry into a target cell. It has been shown virus binds via the receptor binding domain of its spike protein to dipeptidyl peptidase 4 (DPP4) on human epithelial and endothelial cells (see e.g., Raj et al 2013, Nature 495: 251-256). MERS-CoV receptor binding domain consists of a core and a receptor binding subdomain that interacts with DPP4 (see e.g., Lu et al 2013, Nature 500: 227-231).

The MERS-CoV spike protein is a 1353 amino acid type I membrane glycoprotein which assembles into trimers that constitute the spikes or peplomers on the surface of the enveloped MERS coronavirus particle. The protein has two essential functions, host receptor binding and membrane fusion, which are attributed to the N-terminal (S1, amino acid residues 1-751) and C-terminal (S2, amino acid residues 752-1353) halves of the S protein. MERS-CoV-S binds to its cognate receptor, dipeptidyl peptidase 4 (DPP4) via about 230-amino acid long receptor binding domain (RBD) present in the S1 subunit. MERS-CoV RBD is located within the residues 358-588 of the spike protein (see e.g., Mou et al (2013) J. Virology vol 87, pages 9379-9383). The amino acid sequence of full-length MERS-CoV spike protein is exemplified by the amino acid sequence of spike protein of MERS-CoV isolate EMC/2012 provided in GenBank as accession number AFS88936.1 (SEQ ID NO: 8).

In some embodiments of any of the aspects, the target analyte (e.g., protein, glycoprotein, or nucleic acid) comprises at least a portion of MERS-CoV, (see e.g., complete genome, NCBI Reference Sequence: NC_019843.3, isolate HCoV-EMC/2012).

In some embodiments of any of the aspects, the target analyte comprises one of SEQ ID NOs: 7-9 (MERS-CoV S gene). In some embodiments of any of the aspects, the target nucleic acid comprises SEQ ID NO: 7 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 7 that maintains the same function or a codon-optimized version of SEQ ID NO: 7. In some embodiments of any of the aspects, the target nucleic acid comprises SEQ ID NO: 7 or a nucleic acid sequence that is at least 95% identical to SEQ ID NO: 7 that maintains the same function or a codon-optimized version of SEQ ID NO: 7.

In some embodiments of any of the aspects, the target polypeptide comprises SEQ ID NO: 8 or SEQ ID NO: 9 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 8-9 that maintains the same function. In some embodiments of any of the aspects, the target polypeptide comprises one of SEQ ID NO: 8-9 or an amino acid sequence that is at least 95% identical to one of SEQ ID NO: 8-9 that maintains the same function.

SEQ ID NO: 7, S protein [MERS, Human betacoronavirus 2c EMC/2012], NCBI Reference Sequence: NC_019843.3, REGION: 21456-25514, 4059 bp

atgatacactcagtgtttctactgatgttcttgttaacac
ctacagaaagttacgttgatgtagggccagattctgttaa
gtctgcttgtattgaggttgatatacaacagactttcttt
gataaaacttggcctaggccaattgatgtttctaaggctg
acggtattatataccctcaaggccgtacatattctaacat
aactatcacttatcaaggtctttttccctatcagggagac
catggtgatatgtatgtttactctgcaggacatgctacag
gcacaactccacaaaagttgtttgtagctaactattctca
ggacgtcaaacagtttgctaatgggtttgtcgtccgtata
ggagcagctgccaattccactggcactgttattattagcc
catctaccagcgctactatacgaaaaatttaccctgcttt
tatgctgggttcttcagttggtaatttctcagatggtaaa
atgggccgcttcttcaatcatactctagttcttttgcccg
atggatgtggcactttacttagagctttttattgtattct
agagcctcgctctggaaatcattgtcctgctggcaattcc
tatacttcttttgccacttatcacactcctgcaacagatt
gttctgatggcaattacaatcgtaatgccagtctgaactc
ttttaaggagtattttaatttacgtaactgcacctttatg
tacacttataacattaccgaagatgagattttagagtggt
ttggcattacacaaactgctcaaggtgttcacctcttctc
atctcggtatgttgatttgtacggcggcaatatgtttcaa
tttgccaccttgcctgtttatgatactattaagtattatt
ctatcattcctcacagtattcgttctatccaaagtgatag
aaaagcttgggctgccttctacgtatataaacttcaaccg
ttaactttcctgttggatttttctgttgatggttatatac
gcagagctatagactgtggttttaatgatttgtcacaact
ccactgctcatatgaatccttcgatgttgaatctggagtt
tattcagtttcgtctttcgaagcaaaaccttctggctcag
ttgtggaacaggctgaaggtgttgaatgtgatttttcacc
tcttctgtctggcacacctcctcaggtttataatttcaag
cgtttggtttttaccaattgcaattataatcttaccaaat
tgctttcacttttttctgtgaatgattttacttgtagtca
aatatctccagcagcaattgctagcaactgttattcttca
ctgattttggattacttttcatacccacttagtatgaaat
ccgatctcagtgttagttctgctggtccaatatcccagtt
taattataaacagtccttttctaatcccacatgtttgatt
ttagcgactgttcctcataaccttactactattactaagc
ctcttaagtacagctatattaacaagtgctctcgtcttct
ttctgatgatcgtactgaagtacctcagttagtgaacgct
aatcaatactcaccctgtgtatccattgtcccatccactg
tgtgggaagacggtgattattataggaaacaactatctcc
acttgaaggtggtggctggcttgttgctagtggctcaact
gttgccatgactgagcaattacagatgggctttggtatta
cagttcaatatggtacagacaccaatagtgtttgccccaa
gcttgaatttgctaatgacacaaaaattgcctctcaatta
ggcaattgcgtggaatattccctctatggtgtttcgggcc
gtggtgtttttcagaattgcacagctgtaggtgttcgaca
gcagcgctttgtttatgatgcgtaccagaatttagttggc
tattattctgatgatggcaactactactgtttgcgtgctt
gtgttagtgttcctgtttctgtcatctatgataaagaaac
taaaacccacgctactctatttggtagtgttgcatgtgaa
cacatttcttctaccatgtctcaatactcccgttctacgc
gatcaatgcttaaacggcgagattctacatatggccccct
tcagacacctgttggttgtgtcctaggacttgttaattcc
tctttgttcgtagaggactgcaagttgcctcttggtcaat
ctctctgtgctcttcctgacacacctagtactctcacacc
tcgcagtgtgcgctctgttccaggtgaaatgcgcttggca
tccattgcttttaatcatcctattcaggttgatcaactta
atagtagttattttaaattaagtatacccactaatttttc
ctttggtgtgactcaggagtacattcagacaaccattcag
aaagttactgttgattgtaaacagtacgtttgcaatggtt
tccagaagtgtgagcaattactgcgcgagtatggccagtt
ttgttccaaaataaaccaggctctccatggtgccaattta
cgccaggatgattctgtacgtaatttgtttgcgagcgtga
aaagctctcaatcatctcctatcataccaggttttggagg
tgactttaatttgacacttctagaacctgtttctatatct
actggcagtcgtagtgcacgtagtgctattgaggatttgc
tatttgacaaagtcactatagctgatcctggttatatgca
aggttacgatgattgcatgcagcaaggtccagcatcagct
cgtgatcttatttgtgctcaatatgtggctggttacaaag
tattacctcctcttatggatgttaatatggaagccgcgta
tacttcatctttgcttggcagcatagcaggtgttggctgg
actgctggcttatcctcctttgctgctattccatttgcac
agagtatcttttataggttaaacggtgttggcattactca
acaggttctttcagagaaccaaaagcttattgccaataag
tttaatcaggctctgggagctatgcaaacaggcttcacta
caactaatgaagcttttcagaaggttcaggatgctgtgaa
caacaatgcacaggctctatccaaattagctagcgagcta
tctaatacttttggtgctatttccgcctctattggagaca
tcatacaacgtcttgatgttctcgaacaggacgcccaaat
agacagacttattaatggccgtttgacaacactaaatgct
tttgttgcacagcagcttgttcgttccgaatcagctgctc
tttccgctcaattggctaaagataaagtcaatgagtgtgt
caaggcacaatccaagcgttctggattttgcggtcaaggc
acacatatagtgtcctttgttgtaaatgcccctaatggcc
tttacttcatgcatgttggttattaccctagcaaccacat
tgaggttgtttctgcttatggtctttgcgatgcagctaac
cctactaattgtatagcccctgttaatggctactttatta
aaactaataacactaggattgttgatgagtggtcatatac
tggctcgtccttctatgcacctgagcccattacctccctt
aatactaagtatgttgcaccacaggtgacataccaaaaca
tttctactaacctccctcctcctcttctcggcaattccac
cgggattgacttccaagatgagttggatgagtttttcaaa
aatgttagcaccagtatacctaattttggttccctaacac
agattaatactacattactcgatcttacctacgagatgtt
gtctcttcaacaagttgttaaagcccttaatgagtcttac
atagaccttaaagagcttggcaattatacttattacaaca
aatggccgtggtacatttggcttggtttcattgctgggct
tgttgccttagctctatgcgtcttcttcatactgtgctgc
actggttgtggcacaaactgtatgggaaaacttaagtgta
atcgttgttgtgatagatacgaggaatacgacctcgagcc
gcataaggttcatgttcac

SEQ ID NO: 8, S protein [MERS, Human betacoronavirus 2c EMC/2012], GenBank: AFS88936.1 1353 aa

MIHSVFLLMFLLTPTESYVDVGPDSVKSACIEVDIQQTFF
DKTWPRPIDVSKADGIIYPQGRTYSNITITYQGLFPYQGD
HGDMYVYSAGHATGTTPQKLFVANYSQDVKQFANGFVVRI
GAAANSTGTVIISPSTSATIRKIYPAFMLGSSVGNFSDGK
MGRFFNHTLVLLPDGCGTLLRAFYCILEPRSGNHCPAGNS
YTSFATYHTPATDCSDGNYNRNASLNSFKEYFNLRNCTFM
YTYNITEDEILEWFGITQTAQGVHLFSSRYVDLYGGNMFQ
FATLPVYDTIKYYSIIPHSIRSIQSDRKAWAAFYVYKLQP
LTFLLDFSVDGYIRRAIDCGFNDLSQLHCSYESFDVESGV
YSVSSFEAKPSGSVVEQAEGVECDFSPLLSGTPPQVYNFK
RLVFTNCNYNLTKLLSLFSVNDFTCSQISPAAIASNCYSS
LILDYFSYPLSMKSDLSVSSAGPISQFNYKQSFSNPTCLI
LATVPHNLTTITKPLKYSYINKCSRLLSDDRTEVPQLVNA
NQYSPCVSIVPSTVWEDGDYYRKQLSPLEGGGWLVASGST
VAMTEQLQMGFGITVQYGTDTNSVCPKLEFANDTKIASQL
GNCVEYSLYGVSGRGVFQNCTAVGVRQQRFVYDAYQNLVG
YYSDDGNYYCLRACVSVPVSVIYDKETKTHATLFGSVACE
HISSTMSQYSRSTRSMLKRRDSTYGPLQTPVGCVLGLVNS
SLFVEDCKLPLGQSLCALPDTPSTLTPRSVRSVPGEMRLA
SIAFNHPIQVDQLNSSYFKLSIPTNFSFGVTQEYIQTTIQ
KVTVDCKQYVCNGFQKCEQLLREYGQFCSKINQALHGANL
RQDDSVRNLFASVKSSQSSPIIPGFGGDFNLTLLEPVSIS
TGSRSARSAIEDLLFDKVTIADPGYMQGYDDCMQQGPASA
RDLICAQYVAGYKVLPPLMDVNMEAAYTSSLLGSIAGVGW
TAGLSSFAAIPFAQSIFYRLNGVGITQQVLSENQKLIANK
FNQALGAMQTGFTTTNEAFQKVQDAVNNNAQALSKLASEL
SNTFGAISASIGDIIQRLDVLEQDAQIDRLINGRLTTLNA
FVAQQLVRSESAALSAQLAKDKVNECVKAQSKRSGFCGQG
THIVSFVVNAPNGLYFMHVGYYPSNHIEVVSAYGLCDAAN
PTNCIAPVNGYFIKTNNTRIVDEWSYTGSSFYAPEPITSL
NTKYVAPQVTYQNISTNLPPPLLGNSTGIDFQDELDEFFK
NVSTSIPNFGSLTQINTTLLDLTYEMLSLQQVVKALNESY
IDLKELGNYTYYNKWPWYIWLGFIAGLVALALCVFFILCC
TGCGTNCMGKLKCNRCCDRYEEYDLEPHKVHVH

SEQ ID NO: 9, MERS-CoV, receptor binding domain (RBD) of the spike protein, 212 aa (see e.g., FIG. 9); corresponds to amino acids (aa) 377-588 of SEQ ID NO: 8

QAEGVECDFSPLLSGTPPQVYNFKRLVFTNCNYNLTKLLS
LFSVNDFTCSQISPAAIASNCYSSLILDYFSYPLSMKSDL
SVSSAGPISQFNYKQSFSNPTCLILATVPHNLTTITKPLK
YSYINKCSRLLSDDRTEVPQLVNANQYSPCVSIVPSTVWE
DGDYYRKQLSPLEGGGWLVASGSTVAMTEQLQMGFGITVQ
YGTDTNSVCPKL

The term “MERS-CoV-S” also includes protein variants of MERS-CoV spike protein isolated from different MERS-CoV isolates, e.g., Jordan-N3/2012, England-Qatar/2012, Al-Hasa_1_2013, Al-Hasa_2_2013, Al-Hasa_3_2013, Al-Hasa_4_2013, Al-Hasa_12, Al-Hasa 15, Al-Hasa 16, Al-Hasa_17, Al-Hasa_18, Al-Hasa 19, Al-Hasa 21, Al-Hasa 25, Bisha_1, Buraidah_1, England 1, Hafr-Al-Batin_1, Hafr-Al-Batin_2, Hafr-Al-Batin_6, Jeddah_1, KFU-HKU 1, KFU-HKU 13, Munich, Qatar3, Qatar4, Riyadh_1, Riyadh_2, Riyadh_3, Riyadh_3, Riyadh 4, Riyadh_5, Riyadh_9, Riyadh_14, Taif 1, UAE, and Wadi-Ad-Dawasir. The term “MERS-CoV-S” includes recombinant MERS-CoV spike protein or a fragment thereof.

The systems and methods herein also include target molecules associated with viruses other than coronaviruses. Non limiting examples of target molecules include antigens associated with seasonal influenza, highly pathogenic influenza, HIV, Ebola virus, herpes simplex virus 1, herpes simplex virus 2, human papilloma viruses, Marburg virus, Lassa virus, respiratory syncytial virus (RSV).

In one embodiment, one of the binding agents bind the SARS-CoV-2 spike (S) protein using the human angiotensin converting enzyme (ACE). In a particular embodiment, the ACE protein binds the receptor binding domain (RBD) of the S protein.

In a particular embodiment, at least one target analyte is a virus and more particularly, a coronavirus such as a betacoronavirus and even more particularly, SARS-CoV-1.

In one embodiment, the first and second binding agents bind different epitopes on the SARS-CoV-1 spike (S) protein. In a particular embodiment, at least one of the epitopes is within the receptor binding domain (RBD) of the S1 protein.

In one embodiment, one of the binding agents bind the SARS-CoV-1 spike (S) protein using the human angiotensin converting enzyme (ACE). In a particular embodiment, the ACE protein binds the receptor binding domain (RBD) of the S protein.

In a particular embodiment, at least one target analyte is a virus and more particularly a rhinovirus. In one embodiment, the first and second binding agents bind one of the 4 possible capsid proteins of the rhinovirus.

In a particular embodiment, at least one target analyte is a virus and more particularly common human coronaviruses, including types 229E, NL63, OC43, and HKU1. In one embodiment, the first and second binding agents bind the spike protein, the membrane protein, the hemagglutinin protein, the envelope or envelope protein of common human coronaviruses (e.g., types 229E, NL63, OC43, and HKU1).

In a particular embodiment, at least one target analyte is a virus and more particularly respiratory syncytial virus (RSV), parainfluenza (PIV), or HIN1. In one embodiment, the first and second binding agents bind the fusion protein, the membrane protein, the hemagglutinin protein, the neuraminidase protein, the envelope or envelope protein of respiratory syncytial virus (RSV) parainfluenza (PIV), or HIN1.

In a particular embodiment, at least one target analyte is a virus and more particularly human metapneumovirus. In one embodiment, the first and second binding agents bind the fusion protein, the SH protein, the matrix protein, the glycoprotein, the envelope or envelope protein of human metapneumovirus.

In a particular embodiment, at least one target analyte is a virus and more particularly human immunodeficiency virus (HIV). In one embodiment, the first and second binding agents bind the MIHC protein, the p17 matrix protein, the gp120 docking glycoprotein, the gp41 transmembrane glycoprotein, the envelope, or envelope protein of human immunodeficiency virus (HIV).

In a particular embodiment, at least one target analyte is a virus and more particularly Ebola virus. In one embodiment, the first and second binding agents bind the glycoprotein, the matrix protein, the nucleoprotein, the envelope or envelope protein of Ebola virus.

In a particular embodiment, at least one target analyte is a virus and more particularly Marburg virus. In one embodiment, the first and second binding agents bind the glycoprotein, the VP40 matrix protein, the nucleoprotein, the envelope or envelope protein of Marburg virus.

In a particular embodiment, at least one target analyte is a virus and more particularly Lassa virus. In one embodiment, the first and second binding agents bind the glycoprotein 1, the glycoprotein 2, the large protein, the zinc protein, the stable signal peptide (SSP), the nucleoprotein, the envelope or envelope protein of Lassa virus.

In a particular embodiment, at least one target analyte is a parasite and more particularly a malaria Plasmodium species (e.g., P. falciparum, P. malariae, P. vivax, P. ovale, or P. knowlesi). In one embodiment, the first and second binding agents bind the TRAP protein, the SPECT protein, the MAEBL protein, a PPLP protein, a LSA protein, the STARP protein, the CS protein, the SALSA protein, the SPATR protein, the PxSR protein, or the PfEMP3 protein of a malaria Plasmodium species.

In certain embodiments, the one or more target analytes or pathogens are found in biologic samples from animals other than humans, e.g., West-Nile virus and zoonotic pathogens in bats.

In some embodiments of any of the aspects, the target analyte is an analyte (e.g., protein, glycoprotein, nucleic acid) from an RNA virus or a DNA virus. As used herein, the term “RNA virus” refers to a virus comprising an RNA genome. In some embodiments of any of the aspects, the RNA virus is a double-stranded RNA virus, a positive-sense RNA virus, a negative-sense RNA virus, or a reverse transcribing virus (e.g., retrovirus). As used herein, the term “DNA virus” refers to a virus comprising a DNA genome. In some embodiments of any of the aspects, the DNA virus is a Group I (dsDNA) virus, a Group II (ssDNA) virus, or a Group VII (dsDNA-RT) virus.

In some embodiments of any of the aspects, the RNA virus is a Group III (i.e., double stranded RNA (dsRNA)) virus. In some embodiments of any of the aspects, the Group III RNA virus belongs to a viral family selected from the group consisting of: Amalgaviridae, Birnaviridae, Chrysoviridae, Cystoviridae, Endomaviridae, Hypoviridae, Megabirnaviridae, Partitiviridae, Picobimaviridae, Reoviridae (e.g., Rotavirus), Totiviridae, Quadriviridae. In some embodiments of any of the aspects, the Group III RNA virus belongs to the Genus Botybimavirus. In some embodiments of any of the aspects, the Group III RNA virus is an unassigned species selected from the group consisting of: Botrytis porri RNA virus 1, Circulifer tenellus virus 1, Colletotrichum camelliae filamentous virus 1, Cucurbit yellows associated virus, Sclerotinia sclerotiorum debilitation-associated virus, and Spissistilus festinus virus 1.

In some embodiments of any of the aspects, the RNA virus is a Group IV (i.e., positive-sense single stranded (ssRNA)) virus. In some embodiments of any of the aspects, the Group IV RNA virus belongs to a viral order selected from the group consisting of: Nidovirales, Picomavirales, and Tymovirales. In some embodiments of any of the aspects, the Group IV RNA virus belongs to a viral family selected from the group consisting of: Arteriviridae, Coronaviridae (e.g., Coronavirus, SARS-CoV), Mesoniviridae, Roniviridae, Dicistroviridae, Iflaviridae, Marnaviridae, Picornaviridae (e.g., Poliovirus, Rhinovirus (a common cold virus), Hepatitis A virus), Secoviridae (e.g., sub Comovirinae), Alphaflexiviridae, Betaflexiviridae, Gammaflexiviridae, Tymoviridae, Alphatetraviridae, Alvernaviridae, Astroviridae, Barnaviridae, Benyviridae, Bromoviridae, Caliciviridae (e.g., Norwalk virus), Carmotetraviridae, Closteroviridae, Flaviviridae (e.g., Yellow fever virus, West Nile virus, Hepatitis C virus, Dengue fever virus, Zika virus), Fusariviridae, Hepeviridae, Hypoviridae, Leviviridae, Luteoviridae (e.g., Barley yellow dwarf virus), Polycipiviridae, Namaviridae, Nodaviridae, Permutotetraviridae, Potyviridae, Sarthroviridae, Statovirus, Togaviridae (e.g., Rubella virus, Ross River virus, Sindbis virus, Chikungunya virus), Tombusviridae, and Virgaviridae. In some embodiments of any of the aspects, the Group IV RNA virus belongs to a viral genus selected from the group consisting of: Bacillariomavirus, Dicipivirus, Labyrnavirus, Sequiviridae, Blunervirus, Cilevirus, Higrevirus, Idaeovirus, Negevirus, Ourmiavirus, Polemovirus, Sinaivirus, and Sobemovirus. In some embodiments of any of the aspects, the Group IV RNA virus is an unassigned species selected from the group consisting of: Acyrthosiphon pisum virus, Bastrovirus, Blackford virus, Blueberry necrotic ring blotch virus, Cadicistrovirus, Chara australis virus, Extra small virus, Goji berry chlorosis virus, Hepelivirus, Jingmen tick virus, Le Blanc virus, Nedicistrovirus, Nesidiocoris tenuis virus 1, Niflavirus, Nylanderia fulva virus 1, Orsay virus, Osedaxjaponicus RNA virus 1, Picalivirus, Plasmopara halstedii virus, Rosellinia necatrix fusarivirus 1, Santeuil virus, Secalivirus, Solenopsis invicta virus 3, Wuhan large pig roundworm virus. In some embodiments of any of the aspects, the Group IV RNA virus is a satellite virus selected from the group consisting of: Family Sarthroviridae, Genus Albetovirus, Genus Aumaivirus, Genus Papanivirus, Genus Virtovirus, and Chronic bee paralysis virus.

In some embodiments of any of the aspects, the RNA virus is a Group V (i.e., negative-sense ssRNA) virus. In some embodiments of any of the aspects, the Group V RNA virus belongs to a viral phylum or subphylum selected from the group consisting of: Negamaviricota, Haploviricotina, and Polyploviricotina. In some embodiments of any of the aspects, the Group V RNA virus belongs to a viral class selected from the group consisting of: Chunqiuviricetes, Ellioviricetes, Insthoviricetes, Milneviricetes, Monjiviricetes, and Yunchangviricetes. In some embodiments of any of the aspects, the Group V RNA virus belongs to a viral order selected from the group consisting of: Articulavirales, Bunyavirales, Goujianvirales, Jingchuvirales, Mononegavirales, Muvirales, and Serpentovirales. In some embodiments of any of the aspects, the Group V RNA virus belongs to a viral family selected from the group consisting of: Amnoonviridae (e.g., Taastrup virus), Arenaviridae (e.g., Lassa virus), Aspiviridae, Bornaviridae (e.g., Boma disease virus), Chuviridae, Cruliviridae, Feraviridae, Filoviridae (e.g., Ebola virus, Marburg virus), Fimoviridae, Hantaviridae, Jonviridae, Mymonaviridae, Nairoviridae, Nyamiviridae, Orthomyxoviridae (e.g., Influenza viruses), Paramyxoviridae (e.g., Measles virus, Mumps virus, Nipah virus, Hendra virus, and NDV), Peribunyaviridae, Phasmaviridae, Phenuiviridae, Pneumoviridae (e.g., RSV and Metapneumovirus), Qinviridae, Rhabdoviridae (e.g., Rabies virus), Sunviridae, Tospoviridae, and Yueviridae. In some embodiments of any of the aspects, the Group V RNA virus belongs to a viral genus selected from the group consisting of: Anphevirus, Arlivirus, Chengtivirus, Crustavirus, Tilapineviridae, Wastrivirus, and Deltavirus (e.g., Hepatitis D virus).

In some embodiments of any of the aspects, the RNA virus is a Group VI RNA virus, which comprise a virally encoded reverse transcriptase. In some embodiments of any of the aspects, the Group VI RNA virus belongs to the viral order Ortervirales. In some embodiments of any of the aspects, the Group VI RNA virus belongs to a viral family or subfamily selected from the group consisting of: Belpaoviridae, Caulimoviridae, Metaviridae, Pseudoviridae, Retroviridae (e.g., Retroviruses, e.g. HIV), Orthoretrovirinae, and Spumaretrovirinae. In some embodiments of any of the aspects, the Group VI RNA virus belongs to a viral genus selected from the group consisting of: Alpharetrovirus (e.g., Avian leukosis virus; Rous sarcoma virus), Betaretrovirus (e.g., Mouse mammary tumour virus), Bovispumavirus (e.g., Bovine foamy virus), Deltaretrovirus (e.g., Bovine leukemia virus; Human T-lymphotropic virus), Epsilonretrovirus (e.g., Walleye dermal sarcoma virus), Equispumavirus (e.g., Equine foamy virus), Felispumavirus (e.g., Feline foamy virus), Gammaretrovirus (e.g., Murine leukemia virus; Feline leukemia virus), Lentivirus (e.g., Human immunodeficiency virus 1; Simian immunodeficiency virus; Feline immunodeficiency virus), Prosimiispumavirus (e.g., Brown greater galago prosimian foamy virus), and Simiispumavirus (e.g., Eastern chimpanzee simian foamy virus). In some embodiments of any of the aspects, the virus is an endogenous retrovirus (ERV; e.g., endogenous retrovirus group W envelope member 1 (ERVWE1); HCP5 (HLA Complex P5); Human teratocarcinoma-derived virus), which are endogenous viral elements in the genome that closely resemble and can be derived from retroviruses.

In some embodiments of any of the aspects, the DNA virus is a Group I (i.e., dsDNA) virus. In some embodiments of any of the aspects, the Group I dsDNA virus belongs to a viral order selected from the group consisting of: Caudovirales; Herpesvirales; and Ligamenvirales. In some embodiments of any of the aspects, the Group I dsDNA virus belongs to a viral family selected from the group consisting of: Adenoviridae (e.g., adenoviruses), Alloherpesviridae, Ampullaviridae, Ascoviridae, Asfarviridae (e.g., African swine fever virus), Baculoviridae, Bicaudaviridae, Clavaviridae, Corticoviridae, Fuselloviridae, Globuloviridae, Guttaviridae, Herpesviridae (e.g., human herpesviruses, Varicella Zoster virus), Hytrosaviridae, Iridoviridae, Lavidaviridae, Lipothrixviridae, Malacoherpesviridae, Marseilleviridae, Mimiviridae, Myoviridae (e.g., Enterobacteria phage T4), Nimaviridae, Nudiviridae, Pandoraviridae, Papillomaviridae, Phycodnaviridae, Plasmaviridae, Podoviridae (e.g., Enterobacteria phage T7), Polydnaviruses, Polyomaviridae (e.g., Simian virus 40, JC virus, BK virus), Poxviridae (e.g., Cowpox virus, smallpox), Rudiviridae, Siphoviridae (e.g., Enterobacteria phage λ), Sphaerolipoviridae, Tectiviridae, Tristromaviridae, and Turriviridae. In some embodiments of any of the aspects, the Group I dsDNA virus belongs to a viral genus selected from the group consisting of: Dinodnavirus, Rhizidiovirus, and Salterprovirus. In some embodiments of any of the aspects, the Group I dsDNA virus belongs to an unassigned viral species selected from the group consisting of: Abalone shriveling syndrome-associated virus, Apis mellifera filamentous virus, Bandicoot papillomatosis carcinomatosis virus, Cedratvirus, Kaumoebavirus, KIs-V, Lentille virus, Leptopilina boulardi filamentous virus, Megavirus, Metallosphaera turreted icosahedral virus, Methanosarcina spherical virus, Mollivirus sibericum virus, Orpheovirus IHUMI-LCC2, Phaeocystis globosa virus, and Pithovirus. In some embodiments of any of the aspects, the Group I dsDNA virus is a virophage selected from the group consisting of: Organic Lake virophage, Ace Lake Mavirus virophage, Dishui Lake virophage 1, Guarani virophage, Phaeocystis globosa virus virophage, Rio Negro virophage, Sputnik virophage 2, Yellowstone Lake virophage 1, Yellowstone Lake virophage 2, Yellowstone Lake virophage 3, Yellowstone Lake virophage 4, Yellowstone Lake virophage 5, Yellowstone Lake virophage 6, Yellowstone Lake virophage 7, and Zamilon virophage 2.

In some embodiments of any of the aspects, the DNA virus is a Group II (i.e., ssDNA) virus. In some embodiments of any of the aspects, the Group II ssDNA virus belongs to a viral family selected from the group consisting of: Anelloviridae, Bacilladnaviridae, Bidnaviridae, Circoviridae, Geminiviridae, Genomoviridae, Inoviridae, Microviridae, Nanoviridae, Parvoviridae, Smacoviridae, and Spiraviridae.

In some embodiments of any of the aspects, the DNA virus is a Group VII (i.e., dsDNA-RT) virus. In some embodiments of any of the aspects, the Group VII dsDNA-RT virus belongs to the Ortervirales order. In some embodiments of any of the aspects, the Group VII dsDNA-RT virus belongs to the Caulimoviridae family or to the Hepadnaviridae family (e.g., Hepatitis B virus). In some embodiments of any of the aspects, the Group VII dsDNA-RT virus belongs to a viral genus selected from the group consisting of: Badnavirus, Caulimovirus, Cavemovirus, Petuvirus, Rosadnavirus, Solendovirus, Soymovirus, Tungrovirus, Avihepadnavirus, and Orthohepadnavirus.

In some embodiments of any of the aspects, the target analyte is from a coronavirus. The scientific name for coronavirus is Orthocoronavirinae or Coronavirinae. Coronaviruses belong to the family of Coronaviridae, order Nidovirales, and realm Riboviria. They are divided into alphacoronaviruses and betacoronaviruses which infect mammals—and gammacoronaviruses and deltacoronaviruses which primarily infect birds. Non limiting examples of alphacoronaviruses include: Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, and Feline Infectious Peritonitis Virus (FIPV, also referred to as Feline Infectious Hepatitis Virus). Non limiting examples of betacoronaviruses include: Betacoronavirus 1 (e.g., Bovine Coronavirus, Human coronavirus OC43), Human coronavirus HKU1, Murine coronavirus (also known as Mouse hepatitis virus (MHV)), Pipistrellus bat coronavirus HKU5, Rousettus bat coronavirus HKU9, Severe acute respiratory syndrome-related coronavirus (e.g., SARS-CoV, SARS-CoV-2), Tylonycteris bat coronavirus HKU4, Middle East respiratory syndrome (MERS)-related coronavirus, and Hedgehog coronavirus 1 (EriCoV). Non limiting examples of gammacoronaviruses include: Beluga whale coronavirus SW1, and Infectious bronchitis virus. Non limiting examples of deltacoronaviruses include: Bulbul coronavirus HKU11, and Porcine coronavirus HKU15.

In some embodiments of any of the aspects, the target nucleic acid is a nucleic acid (see e.g., FIG. 15A-15F). In some embodiments of any of the aspects, the target nucleic acid is a viral nucleic acid, e.g., a viral DNA or RNA genome or a viral RNA transcript.

Disclosed herein is a binding assay that serves as a component of the systems and methods disclosed herein. The purpose of the binding assay is to bind the at least one target analyte or target molecule and produce a detectable signal.

In one embodiment, the binding assay utilizes standard electrophoretic and immunodiagnostic techniques, including immunoassays such as competition, direct reaction, or sandwich-type assays. Such assays include, but are not limited to, Western blots, agglutination tests, enzyme-labeled and mediated immunoassays (e.g. ELISAs), biotin/avidin type assays, radioimmunoassays or the like. In one embodiment, the binding assay utilizes a sequence-specific endonuclease and a guide nucleic acid, a collateral nucleic acid, and/or a detection nucleic acid.

The specific recognition of the target molecule is achieved by using at least one binding agent. Binding agents suitable for use any molecule or molecules which are capable of association or binding to the one or more target analytes or molecules. The binding agent has at least one binding sites specific for the target analyte.

In one embodiment, the at least one binding agent is selected from an aptamer, antibody, receptor ligand, protein, or molecularly imprinted polymer.

In one embodiment, the specific binding agent is an aptamer of approximately 10-15 kDa in size (20-45 nucleotides), binds its target molecule with at least micromolar affinity, and discriminates against closely related target molecules.

In one embodiment, the specific binding agent is an aptamer of approximately 10-15 kDa in size (20-45 nucleotides), binds its target molecule with at least nanomolar affinity, and/or discriminates against closely related target molecules.

In a particular embodiment, the binding agent is an aptamer wherein the Kd of aptamer to the target molecule is 10 nM or less, or 5 nM or less and can be as low as 100 μM.

In a particular embodiment, the system includes a two binding agent assay, wherein the first and second binding agents are selected from an antibody (e.g., a monoclonal antibody) and an aptamer or a combination thereof. In one embodiment, the first binding agent is an aptamer, and the second binding agent is an antibody (e.g., a monoclonal antibody). In one embodiment, the first binding agent is an antibody (e.g., a monoclonal antibody), and the second binding agent is an aptamer.

In a particular embodiment, the system includes a two binding agent assay, wherein the first and second binding agents are selected from antibodies (e.g., a monoclonal antibodies). In one embodiment, the first binding agent is an antibody, and the second binding agent is an antibody (e.g., a monoclonal antibody) where the antibodies are the same or different or wherein the target for the antibodies in the same or different.

In certain embodiment, the binding assay including a first and second binding agent, wherein the first binding agent binds to a first site on the target analyte, and the second binding agent binds to a second (different) site of the target analyte or molecule.

In certain embodiment, the binding assay including a first and second binding agent, wherein the first binding agent binds to a first site on the target analyte, and the second binding agent binds to a same site of the target analyte. As there are excess copies of the site on the target, both first and second binding agents can bind the target.

In a particular embodiment, the affinity of the first binding agent for the first epitope is greater than the affinity of the second binding agent for the second epitope. The ratio of the Kd of the first epitope to the Kd of the second epitope can range from 1:10,000 to 10,000:1.

In a particular embodiment, the first binding agent is an aptamer, and the second binding agent is an antibody and more particularly, a detectably labeled antibody.

In one embodiment, the antibody is combined or linked with an enzyme (e.g., glucose oxidase) in a fixed ratio of whole numbers (e.g., at least 1, 2, 3, 4, 5 or more enzymes to 1 antibody).

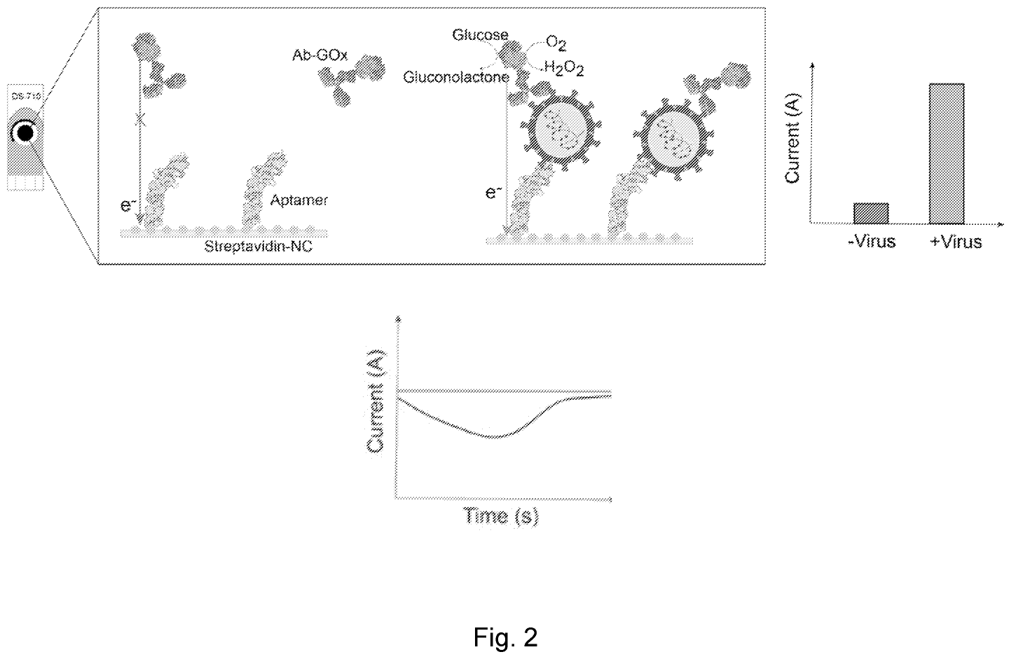

In a particular embodiment, the antibody is combined with glucose oxidase to provide an antibody-GOx conjugate. In another embodiment alternate conjugate strategies using chemical linkers for site specific conjugation to GOx are utilized, e.g., a non-cleavable thioether and peptide linkage. As shown in FIG. 2, the aptamer captures the target analyte (e.g., viral antigen) and Ab-GOx binds only if viral antigen is present. On application of a constant potential, GOx oxidizes glucose, transfers an electron to oxygen, produces hydrogen peroxide, and generates a current output via an electrode that reacts with hydrogen peroxide.

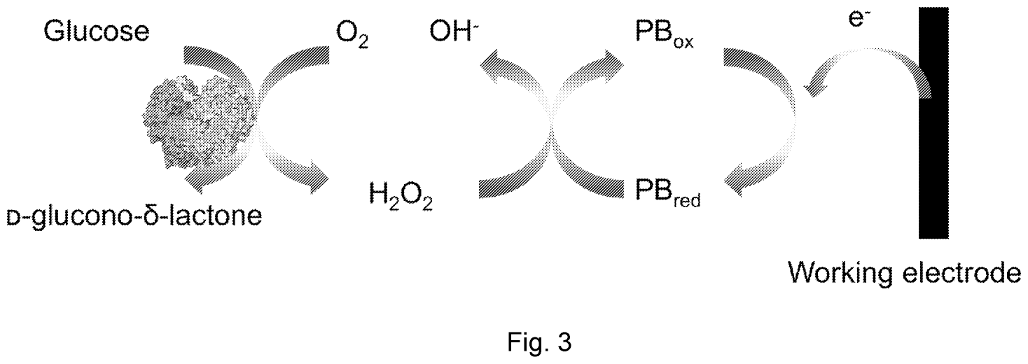

Glucose oxidase (Enzyme Commission number (EC) 1.1.3.4) catalyzes the oxidation of beta-D-glucose to D-glucono-delta-lactone, by utilizing molecular oxygen as an electron acceptor with simultaneous production of hydrogen peroxide (see e.g., FIG. 3). Glucose oxidase functions as a homodimer. Glucose oxidase can also be referred to as beta-D-glucose: oxygen 1-oxidoreductase; notatin; glucose oxyhydrase; corylophyline; penatin; glucose aerodehydrogenase; microcid; beta-D-glucose oxidase; D-glucose oxidase; D-glucose-1-oxidase; beta-D-glucose:quinone oxidoreductase; glucose oxyhydrase; deoxin-1; GOD; or GOx. In some embodiments of any of the aspects, the glucose oxidase is a microbial (e.g., fungal or bacterial) glucose oxidase. In some embodiments of any of the aspects, the glucose oxidase is from Aspergillus niger (see e.g., any of the sequences available under UniProtKB-P13006 (GOX ASPNG) or NCBI gene IDs: 37106576, 4977376, 4985693, 4984787, or 4981316 or any linked orthologs or homologs; see e.g., SEQ ID NO: 10), Penicillium chrysogenum (also known as Penicillium notatum; see e.g., any of the sequences available under UniProtKB-K9L4P7 (K9L4P7_PENCH) or any linked orthologs or homologs), or Penicillium amagasakiense (see e.g., any of the sequences available under UniProtKB-P81156 (GOX PENAG) or any linked orthologs or homologs). See e.g., Raba et al., “Glucose Oxidase as an Analytical Reagent,” Critical Reviews in Analytical Chemistry, 25(1):1-42 (1995), the contents of which are incorporated herein by reference in its entirety.

In some embodiments of any of the aspects, the glucose oxidase comprises SEQ ID NO: 10 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 10 that maintains the same function (e.g., glucose oxidation and/or production of hydrogen peroxide). In some embodiments of any of the aspects, the glucose oxidase comprises SEQ ID NO: 10 or an amino acid sequence that is at least 95% identical to SEQ ID NO: 10 that maintains the same function (e.g., glucose oxidation and/or production of hydrogen peroxide).

SEQ ID NO: 10, Glucose oxidase, precursor, Aspergillus niger, UniProtKB-P13006, 605 amino acids (aa)

MQTLLVSSLVVSLAAALPHYIRSNGIEASLLTDPKDVSGR
TVDYIIAGGGLTGLTTAARLTENPNISVLVIESGSYESDR
GPIIEDLNAYGDIFGSSVDHAYETVELATNNQTALIRSGN
GLGGSTLVNGGTWTRPHKAQVDSWETVFGNEGWNWDNVAA
YSLQAERARAPNAKQIAAGHYFNASCHGVNGTVHAGPRDT
GDDYSPIVKALMSAVEDRGVPTKKDFGCGDPHGVSMFPNT
LHEDQVRSDAAREWLLPNYQRPNLQVLTGQYVGKVLLSQN
GTTPRAVGVEFGTHKGNTHNVYAKHEVLLAAGSAVSPTIL
EYSGIGMKSILEPLGIDTVVDLPVGLNLQDQTTATVRSRI
TSAGAGQGQAAWFATFNETFGDYSEKAHELLNTKLEQWAE
EAVARGGFHNTTALLIQYENYRDWIVNHNVAYSELFLDTA
GVASFDVWDLLPFTRGYVHILDKDPYLHHFAYDPQYFLNE
LDLLGQAAATQLARNISNSGAMQTYFAGETIPGDNLAYDA
DLSAWTEYIPYHFRPNYHGVGTCSMMPKEMGGVVDNAARV
YGVQGLRVIDGSIPPTQMSSHVMTVFYAMALKISDAILED
YASMQ

In a particular embodiment, the antibody is combined with oxidases to provide an antibody-Ox conjugate made from galactose oxidase (see e.g., EC 1.1.3.9); D-glucose:D-fructose oxidoreductase (see e.g., EC 1.1.99.28); or cellobiose oxidase (see e.g., EC 1.1.3.25).

In a particular embodiment, the antibody is combined with dehydrogenases to provide an antibody-DH conjugate made from glucose dehydrogenase (see e.g., EC 1.1.1.47); glucose 6-phosphate dehydrogenase (see e.g., EC 1.1.1.49); fructose dehydrogenase (see e.g., EC 1.1.99.11); sucrose dehydrogenase (also known as glucoside 3-dehydrogenase; see e.g., EC 1.1.99.13); glucoside dehydrogenase (see e.g., EC 1.1.99.13); alcohol dehydrogenase (see e.g., EC 1.1.1.1); sorbitol dehydrogenase (see e.g., EC 1.1.99.21); lactate dehydrogenase (see e.g., EC 1.1.1.27); or malate dehydrogenase (see e.g., 1.1.1.37).

In embodiments of this two binding agent assay, the first and second binding agents may be specific to at least one viral antigen associated with a coronavirus or other virus of interest, e.g., the S protein of a coronavirus or a subunit, fragment or epitope thereof. The at least one viral antigen may be the S-1 subunit of a betacoronavirus or one or more epitopes of the same, and more particularly, a C-type betacoronavirus such as SARS-CoV-2 or SARS-CoV.

In a particular embodiment, the aptamer is the aptamer disclosed in Song, Y. et al. Discovery of Aptamers Targeting Receptor-Binding Domain of the SARS-CoV-2 Spike Glycoprotein. (2020). doi:10.26434/chemrxiv.12053535.v2, or Song et al., Analytical Chemistry, 2 Jul. 2020, 92(14):9895-9900; the contents of each of which are incorporated herein by reference in their entireties.

In another particular embodiment, the antibody is the antibody disclosed in Yuan, M. et al. A highly conserved cryptic epitope in the receptor-binding domains of SARS-CoV-2 and SARS-CoV. Science 633, eabb7269 (2020); the contents of which are incorporated herein by reference in their entirety.

In another particular embodiment, the first binding agent is an aptamer, antibody, or protein bound to a test strip.

In another particular embodiment, the first binding agent is an aptamer bound to a test strip and more particularly, a hydrophilic membrane such as a nitrocellulose membrane.

In another particular embodiment, the first binding agent is an aptamer, antibody, or protein bound to a test strip via a polymeric membrane located on the strip

In another particular embodiment, the first binding agent is an aptamer, antibody, or protein bound to a test strip via a hydrophilic membrane such as a nitrocellulose membrane located on the strip.

In another particular embodiment, the first binding agent is an aptamer, antibody, or protein bound to a test strip via a hydrophilic membrane such as a nitrocellulose membrane which is placed directly above the electrode(s) or between two electrodes on the strip.

In another particular embodiment, the first binding agent is an aptamer, antibody, or protein bound to a test strip via a hydrophilic membrane such as a nitrocellulose membrane which is secured to the strip above the electrode(s) or between two electrodes.

In another particular embodiment, the first binding agent is an aptamer, antibody, or protein bound to a test strip via a hydrophilic membrane wherein the membrane also collects the biological sample(s) and provides a sink area to flow the sample from one location on the membrane to another.

In another particular embodiment, the first binding agent is an aptamer, antibody, or protein bound to a test strip via a hydrophilic membrane such as a nitrocellulose membrane wherein the membrane also collects the biological sample(s) and provides a sink area to flow the sample from one location on the membrane to another.

In some embodiments of any of the aspects, the target analyte is an antibody (e.g., IgG, IgM, and IgA). In some embodiments of any of the aspects, the first binding agent is a protein bound to a test strip, wherein the target analyte (e.g., antibody) specifically binds to the protein. In some embodiments of any of the aspects, the first binding agent is a viral protein (e.g., Flu H1N1 HA or NA; SARS-CoV-2 S spike protein). In some embodiments of any of the aspects, the second binding agent is an aptamer or antibody that specifically binds to the target analyte (e.g., antibody). In some embodiments of any of the aspects, the second binding agent is an anti-antibody antibody. In some embodiments of any of the aspects, the second binding agent is an anti-antibody antibody linked to glucose oxidase. In some embodiments of any of the aspects, the second binding agent is an anti-IgG, anti-IgM, or anti-IgA antibody. In some embodiments of any of the aspects, the second binding agent is an anti-human-IgG, anti-human-IgM, or anti-human-IgA antibody.

Antibody reagents specific for the targets described herein, e.g., Influenza neuraminidase protein, Influenza hemagglutinin protein, SARS-CoV-2 spike or membrane proteins, glucose oxidase, are known in the art. For example, such reagents are readily commercially available. In some embodiments of any of the aspects, an antibody reagent specific for a target described herein (e.g., that binds specifically to a viral antigen, such as Flu HA or NA, or SARS-CoV-2 spike protein or membrane protein, or glucose oxidase) can be an antibody reagent comprising one or more (e.g., one, two, three, four, five, or six) CDRs of any one of the antibodies recited in Table 2. In some embodiments of any of the aspects, an antibody reagent specific for a target described herein (e.g., that binds specifically to a viral antigen, such as Flu HA or NA, or SARS-CoV-2 spike protein or membrane protein, or glucose oxidase) can be an antibody reagent comprising the six CDRs of any one of the antibodies recited in Table 2. In some embodiments of any of the aspects, an antibody reagent specific for a target described herein (e.g., that binds specifically to a viral antigen, such as Flu HA or NA, or SARS-CoV-2 spike protein or membrane protein, or glucose oxidase) can be an antibody reagent comprising the three heavy chain CDRs of any one of the antibodies recited in Table 2. In some embodiments of any of the aspects, an antibody reagent specific for a target described herein (e.g., that binds specifically to a viral antigen, such as Flu HA or NA, or SARS-CoV-2 spike protein or membrane protein, or glucose oxidase) can be an antibody reagent comprising the three light chain CDRs of any one of the antibodies recited in Table 2. In some embodiments of any of the aspects, an antibody reagent specific for a target described herein (e.g., that binds specifically to a viral antigen, such as Flu HA or NA, or SARS-CoV-2 spike protein or membrane protein, or glucose oxidase) can be an antibody reagent comprising the VH and/or VL domains of any one of the antibodies recited in Table 2. In some embodiments of any of the aspects, an antibody reagent specific for a target described herein (e.g., that binds specifically to a viral antigen, such as Flu HA or NA, or SARS-CoV-2 spike protein or membrane protein, or glucose oxidase) can be an antibody reagent comprising the VH and VL domains of any one of the antibodies recited in Table 2. Such antibody reagents are specifically contemplated for use in the methods, systems and/or kits described herein.

TABLE 2
Exemplary Antibody Reagents

| Target | Description | Cat. # |
|---|---|---|
| Flu HA | monoclonal | SINOBIOLOGICAL |
| | | 11684-MM03 |
| Flu HA | monoclonal (C102) | ABCAM ab 128412 |
| Flu NA | polyclonal | ABCAM ab 91646 |
| Flu NA (H1N1, | monoclonal | SINOBIOLOGICAL |
| Swine Flu 2009) | | 11058-MM07 |
| Flu NA | monoclonal (EPR15712) | ABCAM ab 197020 |
| CoV2 spike | polyclonal | ABCAM ab 272504 |
| CoV2 spike | monoclonal (1A9) | ABCAM ab 273433 |
| CoV2 spike | ACE2 (Angiotensin- | ABCAM 273687 |
| | converting enzyme-2)- | |
| | Fc chimera | |
| CoV2 membrane | polyclonal | NOVUS BIOLOGICALS |
| protein | | NB100-56569 |
| glucose oxidase | polyclonal | ABCAM ab181638 |
| glucose oxidase | monoclonal (clone | LSBIO LS-C83029 |
| (Aspergillus niger) | GO14-6.6.1.2) | |

Aptamer reagents specific for the targets described herein, e.g., Influenza neuraminidase protein, Influenza hemagglutinin protein, SARS-CoV-2 spike or membrane proteins, glucose oxidase, are known in the art. For example, such reagents are readily commercially available. In some embodiments, the aptamers are selected from the aptamers described in the following or can be designed using the methods described in the following: Song, Y. et al. Discovery of Aptamers Targeting Receptor-Binding Domain of the SARS-CoV-2 Spike Glycoprotein. (2020). doi: 10.26434/chemrxiv.12053535.v2, or Song et al., Analytical Chemistry, 2 Jul. 2020, 92(14):9895-9900; International Patent Application WO2013183383A1; US Patent Publication US20150167106A1; Gopinath et al., Aptamers that bind to the hemagglutinin of the recent pandemic influenza virus HiN1 and efficiently inhibit agglutination, Acta Biomater. 2013 November; 9(11):8932-41; the contents of each of which are incorporated herein by reference in their entireties. In some embodiments of any of the aspects, the aptamer comprises biotin linked to the 5′ end (5Biosg) or 3′ end (3Biosg) of the aptamer.

In some embodiments of any of the aspects, the aptamer is selected from Table 3. In some embodiments of any of the aspects, the aptamer comprises one of SEQ ID NOs: 11-19 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to one of SEQ ID NOs: 11-19 that maintains the same function (e.g., binding to a target analyte). In some embodiments of any of the aspects, the aptamer comprises one of SEQ ID NOs: 11-19 or a nucleic acid sequence that is at least 95% identical to one of SEQ ID NOs: 11-19 that maintains the same function (e.g., binding to a target analyte).

In some embodiments of any of the aspects, the aptamer comprises one of SEQ ID NOs: 11-12 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to one of SEQ ID NOs: 11-12 that maintains the same function (e.g., binding to a target analyte). In some embodiments of any of the aspects, the aptamer comprises one of SEQ ID NOs: 11-12 or a nucleic acid sequence that is at least 95% identical to one of SEQ ID NOs: 11-12 that maintains the same function (e. g., binding to a target analyte).

TABLE 3
Exemplary Aptamer Reagents

| | SEQ | |
|---|---|---|
| | ID | |
| Target | NO | Sequence |
| H1N1 PR8 | 11 | /5Biosg/AA TTA ACC CTC |
| (e.g., | | ACT AAA GGG CTG AGT |
| Flu HA) | | CTC AAA ACC |
| | | GCA ATA CAC TGG TTG |
| | | TAT GGT CGA ATA AGT |
| | | TAA |
| CoV-2- | 12 | /5Biosg/CAGCACCGACCTTGTGC |
| RBD-1C | | TTTGGGAGTGCTGGTCCAAGGGCG |
| | | TTAATGGACA |
| CoV2-RBD-1 | 13 | /5Biosg/ATCCAGAGTGACGCAGC |
| | | ACCGACCTTGTGCTTTGGGAGTGCT |
| | | GGTCCAAGGGCGTTAATGGACACGG |
| | | TGGCTTAGT |
| CoV2-RBD-2 | 14 | /5Biosg/ATCCAGAGTGACGCAGC |
| | | ATCGAGTGGTGGGCTGGTCGGGTTT |
| | | GGATTCCCTTAGATGCTGGACACGG |
| | | TGGCTTAGT |
| CoV2-RBD-3 | 15 | /5Biosg/ATCCAGAGTGACGCAGC |
| | | ACTGCGTAGGCGCGGCCAATGTGT |
| | | AGGATTGCTCAGGTCTGCTGGACAC |
| | | GGTGGCTTAGT |
| CoV2-RBD-4 | 16 | /5Biosg/ATCCAGAGTGACGCAGC |
| | | ATTTCATCGGGTCCAAAAGGGGCT |
| | | GCTCGGGATTGCGGATATGGACAC |
| | | GGTGGCTTAGT |
| CoV2-RBD-5 | 17 | /5Biosg/ATCCAGAGTGACGCAG |
| | | CAGGACTGCTTAGGATTGCGAAGC |
| | | TGAGGAGCTCCCCCGCCTTGGACA |
| | | CGGTGGCTTAGT |
| CoV2-RBD-6 | 18 | /5Biosg/ATCCAGAGTGACGCAG |
| | | CAGTAGGGGGATTGGCTCCAGGGC |
| | | CTGGCTGACGGTTGCACGTGGACA |
| | | CGGTGGCTTAGT |
| CoV2- | 19 | /5Biosg/ATCCAGAGTGACGCAG |
| RBD-4C | | CATTTCATCGGGTCCAAAAGGGGC |
| | | GCTCGGGATTGCGGATATGGACAC |
| | | GT |

In another aspect, described herein is a system (referred to herein as a nucleic acid detection system; see e.g., FIG. 15A-15F) for detecting at least one target nucleic acid in a biological sample, wherein the system comprises (i) a sequence-specific endonuclease and guide nucleic acid that cleave a collateral nucleic acid upon specific binding of the target nucleic acid to the endonuclease and guide nucleic acid; (ii) a detection nucleic acid that is capable of creating a detectable complex with the cleaved collateral nucleic acid; and (iii) a detection device for detecting the detectable complex, wherein the detection device is an oxidase-based amperometric sensor and the biological sample is present in sweat, salvia, serum, mucus, or blood.

In some embodiments of any of the aspects, the sequence-specific endonuclease is a Cas enzyme. In some embodiments of any of the aspects, the sequence-specific endonuclease is capable of cleaving a collateral nucleic acid when the endonuclease and guide nucleic acid are bound to the target nucleic acid. In some embodiments of any of the aspects, the sequence-specific endonuclease is Cas13a (previously known as C2c2), Cas13b, Cas13c, Cas12a, and/or Csm6. In some embodiments of any of the aspects, the sequence-specific endonuclease is Cas12a or Cas13. See e.g., US Patent Application US20190241954; PCT Patent Application WO2020028729; Chen et al., CRISPR-Cas12a target binding unleashes indiscriminate single-stranded DNase activity, Science. 2018 Apr. 27, 360(6387):436-439; the content of each of which is incorporated herein by reference in its entirety.

In some embodiments of the various aspects described herein, the sequence-specific endonuclease binds to a guide nucleic acid (gNA), e.g., in the presence of the target nucleic acid. As used herein, the terms “guide nucleic acid,” “guide sequence,” “crRNA,” “guide RNA,” or “single guide RNA,” or “gRNA” refers to a polynucleotide comprising any polynucleotide sequence. Generally, the guide nucleic acid sequence is selected to have sufficient complementarity with a target nucleic acid sequence to hybridize with the target nucleic acid sequence and to direct sequence-specific binding of the fusion protein, i.e., sequence-specific endonuclease to the target nucleic acid sequence.

The full-length guide nucleic acid, collateral nucleic acid, and/or detection nucleic acid strand can be any length. For example, the guide nucleic acid, collateral nucleic acid, and/or detection nucleic acid strand can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments of the various aspects described herein, the guide nucleic acid, collateral nucleic acid, and/or detection nucleic acid strand is less than about 75, 50, 45, 40, 35, 30, 25, 20, 15, 12, or fewer nucleotides in length. For example, the guide nucleic acid, collateral nucleic acid, and/or detection nucleic acid strand is 10-30 nucleotides long. In some embodiments of the various aspects described herein, the guide nucleic acid is designed using a guide design tool (e.g., Benchling™; Broad Institute GPP™; CasOFFinder™; CHOPCHOP™; CRISPOR™; Deskgen™; E-CRISP™; Geneious™; GenHub™; GUIDES™ (e.g., for library design); Horizon Discovery™; IDT™; Off_Spotter™; and Synthego™; which are available on the world wide web).

In some embodiments of any of the aspects, the guide nucleic acid is complementary or substantially complementary to at least a portion of the target nucleic acid. In some embodiments of any of the aspects, the detection nucleic acid is complementary or substantially complementary to at least a portion of the cleaved collateral nucleic acid. The term “substantial complementary” or “substantially complementary” as used herein refers both to complete complementarity of binding nucleic acids, in some cases referred to as an identical sequence, as well as complementarity sufficient to achieve the desired binding of nucleic acids. Correspondingly, the term “complementary hybrids” encompasses substantially complementary hybrids.

In some embodiments of any of the aspects, the detection nucleic acid hybridizes to the cleaved collateral nucleic acid. In some embodiments of any of the aspects, the detection nucleic acid does not hybridize to the un-cleaved collateral nucleic acid. In some embodiments of any of the aspects, the detection nucleic acid is linked to a test strip, e.g., at the 5′ or 3′ end of the detection nucleic acid.

In some embodiments of any of the aspects, the collateral nucleic acid is linked to glucose oxidase (see e.g., FIG. 15A), e.g., at the 5′ or 3′ end of the collateral nucleic acid.

In some embodiments of any of the aspects, the system further comprises an aptamer linked to glucose oxidase (see e.g., FIG. 15B-15C). The aptamer can be linked to glucose oxidase using any linkage known in the art, as described further herein. The 3′-end, 5′-end, or an internal region of the aptamer can be linked to the glucose oxidase.

In some embodiments of any of the aspects, the aptamer specifically binds to at least a portion of the cleaved collateral nucleic acid. In some embodiments of any of the aspects, the aptamer binds to a single-stranded portion of the cleaved collateral nucleic acid (see e.g., FIG. 15B). In some embodiments of any of the aspects, the aptamer binds to a double-stranded portion of the cleaved collateral nucleic acid hybridized to the detection nucleic acid (see e.g., FIG. 15C).

In some embodiments of any of the aspects, the system further comprises an antibody linked to glucose oxidase (see e.g., FIG. 15D). The antibody can be linked to glucose oxidase using any linkage known in the art, as described further herein. The N-terminus, C-terminus, or an internal region of the light chain or heavy chain of the antibody can be linked to the glucose oxidase. In some embodiments of any of the aspects, the antibody specifically binds to at least a portion of the cleaved collateral nucleic acid.

In some embodiments of any of the aspects, the collateral nucleic acid is linked to an antibody that specifically binds glucose oxidase (see e.g., FIG. 15E). Antibody reagents specific for glucose oxidase are known in the art. For example, such reagents are readily commercially available (see e.g., Table 2).

In some embodiments of any of the aspects, the collateral nucleic acid is linked to a first member of an affinity pair (see e.g., FIG. 15F). In some embodiments of any of the aspects, the system further comprises glucose oxidase linked to a second member of an affinity pair. In some embodiments of any of the aspects, the first and second members of the affinity pair is selected from the group consisting of: a haptenic or antigenic compound in combination with a corresponding antibody or binding portion or fragment thereof, digoxigenin and anti-digoxigenin; mouse immunoglobulin and goat anti-mouse immunoglobulin; a non-immunological binding pair; biotin and avidin; biotin and streptavidin; a hormone and a hormone-binding protein; thyroxine and cortisol-hormone binding protein; a receptor and a receptor agonist; a receptor and a receptor antagonist; acetylcholine receptor and acetylcholine or an analog thereof, IgG and protein A; lectin and carbohydrate; an enzyme and an enzyme cofactor; an enzyme and an enzyme inhibitor; complementary oligonucleotide pairs capable of forming nucleic acid duplexes; and a first molecule that is negatively charged and a second molecule that is positively charged.

In some embodiments of any of the aspects, the first and second members of the affinity pair is streptavidin and biotin. In some embodiments of any of the aspects, streptavidin is linked to the collateral nucleic acid, and biotin is linked to the glucose oxidase. In some embodiments of any of the aspects, biotin is linked to the collateral nucleic acid, and streptavidin is linked to the glucose oxidase.

Also disclosed herein is a detection device which serves as a component of the systems and methods disclosed herein, wherein the detection device detects the signal produced by the binding assay.

In certain embodiments, the detection device is a portable (e.g., hand-held), battery-powered device.

In one embodiment, the detection device utilized in the systems and methods herein is a glucometer, such as a personal glucose meter (PGM). Conventionally, a PGM is a portable handheld device used to measure blood glucose levels for users with Type I or Type II diabetes. Typically, the user purchases small strips (e.g., about 20-30 mm×about 5-9 mm) that interface with the PGM. The user draws a tiny amount of blood (e.g., a few microliters) from a finger or other area using a lancer, applies a blood droplet sample onto the exposed end of the strip, and then inserts the connector end of the strip into the PGM connector port. A chemical reaction occurs between the blood sample and the chemistry on the strip, which is measured by the PGM to determine the blood glucose level in units of mg/dL or mmol/L, or Kg/L. After measuring blood sugar levels, repeatedly, the used test strip is removed from the PGM, and a new test strip is loaded into the connector port.

In one embodiment, the glucometer in the systems and methods herein in a standard, commercially available, hand-held glucometer. Non-limiting examples of commercially available glucometers include Accu Chek® (ROCHE DIABETES CARE, INC., Indianapolis, Indiana), Van Touch®, Bionime®Presto® (AGAMATRIX, Salem, NH), Wavesense Presto® (AGAMATRIX, Salem, NH), Counter® (ASCENSIA, Basel, Switzerland), CounterPlus® (ASCENSIA, Basel, Switzerland), FreeStyle® (ABBOTT DIABETES CARE INC., Abbott Park, Ill), True® (TRIVIDIA HEALTH, Fort Lauderdale, Florida).

In certain embodiments, the glucometer is a limited-use or disposable glucometer.

A glucometer typically includes a base unit that houses control and test electronics required to test the blood glucose levels in a blood sample. In other embodiments, the glucometer has been modified in one or more ways to enhance functionality for the detection of analytes, either generally or from saliva.

In a particular embodiment, the detection device is a glucometer having a base unit having a test strip slot and a reader configured to analyze a biological sample (e.g., a saliva sample). In one embodiment, the glucometer measures the glucose signal (e.g., quantitatively). The base unit may vary in shape and size. The test strip slot is configured to accept a glucose test strip such as those described herein, which may be removably inserted into the test strip slot. The glucometer may also have a means for storing data and transmitting data.

The glucose measurement may be performed by standard amperometric detection of glucose using glucose oxidase. In this embodiment, the glucose concentration in the biological fluid is converted into a voltage or current signal using a sensor. The sensor uses a platinum and silver electrode to form part of an electric circuit where hydrogen peroxide is electrolyzed. The hydrogen peroxide is produced as a result of the oxidation of glucose on a glucose oxide membrane. The current flowing through the circuit provides a measurement of the concentration of hydrogen peroxide, giving the glucose concentration.

The glucose measurement may be performed by standard amperometric detection of glucose using glucose oxidase. In this embodiment, the glucose concentration in the biological fluid is converted into a voltage or current signal using a sensor. The sensor uses carbon electrode(s) to form part of an electric circuit where hydrogen peroxide is electrolyzed. The hydrogen peroxide is produced as a result of the oxidation of glucose on a glucose oxide membrane. The current flowing through the circuit provides a measurement of the concentration of hydrogen peroxide, giving the glucose concentration.

In certain embodiments, the system comprises one or more signal processing applications or electronic amplifiers in the circuit to amplify the signal.

In one embodiment, the H₂O₂ at these electrodes can be obtained at low applied potential (e.g., about −0.2V versus Ag/AgCl; e.g., Ag/AgCl can be the reference electrode).

In one embodiment, the biological sample is mixed with glucose at a concentration between about 0.01 mM and about 1 M.

In other embodiments, the biological sample is mixed with sucrose, fructose, maltose, galactose, cellulose, or any combination that includes an amylase or invertase at a concentration between 0.01 mM and 1 M. In some embodiments of any of the aspects, the concentration of the glucose, sucrose, fructose, maltose, galactose, or cellulose is at least 0.01 mM, at least 0.02 mM, at least 0.03 mM, at least 0.04 mM, at least 0.05 mM, at least 0.06 mM, at least 0.07 mM, at least 0.08 mM, at least 0.09 mM, at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, or at least 1.0 mM or more.

In one embodiment, the device includes a display unit for displaying the result. The display may display the most recent test and, optionally, previous tests are displayed. In certain embodiments, the glucometer includes a voice control function for ease of use by vision-impaired subjects. The glucometer may include other features unrelated to glucose measurement, e.g., measurement of other physiological functions. The glucose readings displayed on the glucometer can positively correlate to enzyme concentration on the sensor surface which in turn correlates to number of analytes (e.g., viral particles) present in the biological sample.

The glucometer may have a software element. Various software algorithm for glucometers are known.

In one embodiment, the glucometer has a wireless transmitter is configured to communicate a message to a second device, e.g., a mobile device, such as a cellular phone or a tablet computer. In one embodiment, the message is sent to the second device over a short distance communication protocol, e.g., a Bluetooth protocol. The message may also be, for example, a text message or email.

In one embodiment, the glucometer produces a result rapidly after testing has begun, e.g., less than about 5 minutes, less than about 1 minute 30 seconds, less than about 15 seconds or less than about 5 seconds.

The accuracy of the glucometer may vary but is generally does not exceed 20% error and more particularly, does not exceed about 15%, about 10%, about 5% or less than about 5% error, e.g., about 4%, about 3%, about 2% or about 1% or less error. In certain embodiments, cross-sensitivity of the glucometer is reduced or limited based on experimental determination and verification of new correction factors. In one embodiment, the accuracy of the glucometer ranges between about 85% and about 95%.

In one embodiment, the glucometer permits the user to save the latest values of the tests and calculate the average value of glucose for a period of time (e.g., at least two (2) weeks), thereby permitting monitoring overtime.

In certain embodiments, the glucose meter is “displayless” (i.e., does not comprise a display) in order to minimize the complexity and cost of the meter unit. According to this embodiment, the glucose meter is wirelessly enabled and sends the result or readout to a second device, e.g., a cell phone or personal computer.

Optionally, the glucometer also includes a transmitter configured to wirelessly transmit data, encoded within an audio signal, regarding results of the analysis, and a controller configured to facilitate the encoding.

Also disclosed herein is a remote computing device which may be used in the systems and methods herein that are a glucometer, and remote computing device. In one embodiment, the remote communicating device may be, for example, a smartphone or any other suitable device such as a communications device, and which may constitute an output device.

The glucometer transmits the measurements through the transmitter unit, for example over a wireless audio-based channel, to the remote computing device.

The remote computing device may further communicate information to remote devices, such as a central repository device, through a network such as internet- or mobile-based device to a recipient list. For example, the detection device may transmit medical data through the remote computing device. The data may thereafter be communicated to a remote caregiver, e.g., via a computer or handheld device, such as a smartphone.

In this embodiment a software algorithm is disclosed that triggers electrochemical reactions in detection system such that one or more detectable chemical species are the reaction product of a biological sample, test strip and detection device.

In one embodiment, mathematical operations are performed algorithmically localized computing on the detection device such that chemical reactions that afford detectable reaction products proceed between the biological sample, test strip and detection device.

In one embodiment, mathematical operations are performed using cloud computing on servers in a physical location external to the location of the detection device such that chemical reactions that afford detectable reaction products proceed between the biological sample, test strip and detection device.

In one embodiment, a data card containing additional algorithms non initially programmed on detection device is inserted into a data card slot on detection device such that chemical reactions occur that afford detectable reaction products proceed between the biological sample, test strip and detection device.

In one embodiment, a non-transitory computer-readable storage medium is disclosed, encoded with executable instructions for execution by a processor to detect a target analyte.

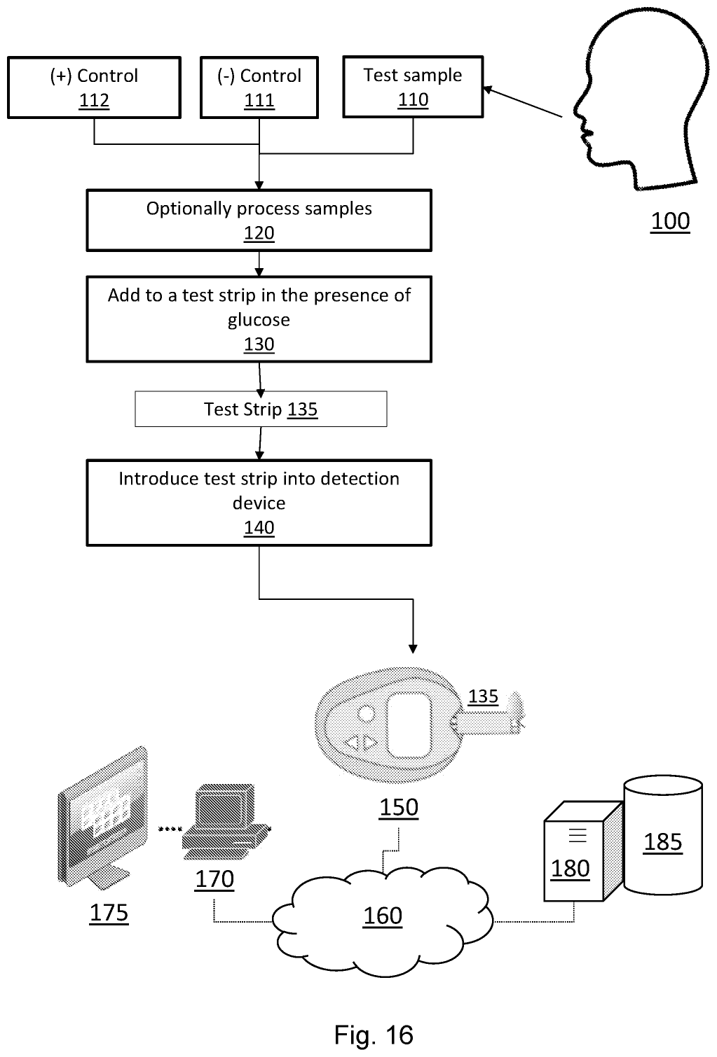

FIG. 16 illustrates an example overview of a system for implementing the technology as described herein. The system includes a detection device 150 (e.g., a glucometer) into which the test strip 135 is introduced (130). Data output from the detection device 150 can be input into a program that may be stored in a database 185.

The computing device 170 and server 180 may be connected by a network 160 and the network 160 may be connected to various other devices, servers, or network equipment for implementing the present disclosure. A computing device 170 may be connected to a display 175. Computing device 170 may be any suitable computing device, including a desktop computer, server (including remote servers), mobile device, or other suitable computing device. In some examples, algorithm(s) as described herein and other software may be stored in database 185 and run on server 180. Additionally, mass spectrometer data (e.g., mass spectra) and data processed or produced by said algorithms or programs (e.g., processed profiles, scores, output tables, etc.), may be stored in database 185.

It should initially be understood that the technology as described herein can be implemented with any type of hardware and/or software, and can be a pre-programmed general purpose computing device. For example, the system can be implemented using a server, a personal computer, a portable computer, a thin client, or any suitable device or devices. The technology as described herein and/or components thereof can be a single device at a single location, or multiple devices at a single, or multiple, locations that are connected together using any appropriate communication protocols over any communication medium such as electric cable, fiber optic cable, or in a wireless manner.

It should also be noted that the technology as described herein is illustrated and discussed herein as having a plurality of modules which perform particular functions. It should be understood that these modules are merely schematically illustrated based on their function for clarity purposes only, and do not necessary represent specific hardware or software. In this regard, these modules can be hardware and/or software implemented to substantially perform the particular functions discussed. Moreover, the modules can be combined together within the technology as described herein, or divided into additional modules based on the particular function desired. Thus, the technology as described herein should not be construed to limit the present technology as disclosed herein, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of these. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA or an ASIC as noted above.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from, or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile or non-transitory memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

Also disclosed are test strips for use in the systems and methods disclosed herein.

In one embodiment, the test strip is a disposable electro-chemical test strip which is contacted with a small volume biological sample. It produces, in conjunction with a test meter, an electrical current which is proportional to the glucose concentration in the biological sample which in turn correlates to a concentration of analyte or the virus.

The test strip includes an insertion portion and an exposed portion. The exposed portion of the test strip is arranged to accept a biological sample (e.g., saliva) from a subject.

In one embodiment, the test strip includes a substrate, which substrate includes at least one test site and two or more electrodes (e.g., working and reference electrodes) and a means for making connection between the electrodes and the meter.

The substrate may be any suitable substrate, for example plastic, ceramic, metal, or polymeric material (e.g., hydrogel).

In one embodiment, the substrate is selected from nitrocellulose (e. g., in membrane or microtiter well form), polyvinylchloride (e. g., sheets or microtiter wells), polystyrene latex (e.g., beads or microtiter plates), polyvinylidene fluoride, diazotized paper, nylon membranes, activated beads or magnetically responsive bead.

In one embodiment, the substrate is an anionic polymer such as a nitrocellulose membrane. In other embodiments, the substrate is sulfonated tetrafluoroethylene, poly(acrylic acid), or poly(2-acrylamido-2-methyl-1-propanesulfonic acid (polyAMPS).

The first binding agent may be immobilized onto the test strip to provide the test site. The binding agent may be an aptamer, antibody, receptor ligand, or molecularly imprinted polymer, as discussed herein. In certain embodiments, the first binding agent (e.g., aptamer) is immobilized directly on the electrode (e.g., screen-printed electrode).

In another embodiment, the first binding agent may be immobilized onto a membrane such as but not limited to nitrocellulose and inserted above electrode in the test strip to provide the test site. The binding agent may be an aptamer, antibody, receptor ligand, protein, or molecularly imprinted polymer, as discussed herein.

In another embodiment, the first binding agent may be immobilized onto a membrane such as but not limited to nitrocellulose and inserted between two electrodes in the test strip to provide the test site. The binding agent may be an aptamer, antibody, receptor ligand, protein, or molecularly imprinted polymer, as discussed herein.

Upon addition of the second binding agent, which is linked to an oxidase (such as but not limited to glucose oxidase), it binds the virus or target analyte which is captured by the first binding agent.

The electrical current is produced by the selective oxidation of glucose, which is catalyzed by two reagents which are pre-coated inside the test strip: (1) an enzyme and (2) a mediator molecule. The enzyme reacts directly with the glucose molecule to produce hydrogen peroxide with the mediator molecule reacts with and transports the electrons to the working electrode.

The enzyme may be, for example, Glucose Oxidase, PQQ-Glucose Dehydrogenase, NAD-Glucose Dehydrogenase, or FAD-Glucose Dehydrogenase.

The mediator molecule may be, for example, ferricyanide; hexacyanoferrate III/hexacyanoferrate II; 1,10-phenanthroline quinone; quinoneimine/phenylenediamine; or an osmium-based mediator.

The free electrons can be moved through a circuit when a voltage is applied between the two electrodes. Each enzyme and mediator molecule can repeat this transfer again and again, if necessary. The amount of charge that moves through the circuit is representative of the glucose level in the system which is reflective of the analyte concentration in the sample.

In a particular embodiment, the glucose oxidase is used as enzyme and the electro-chemical reaction that occurs is shown below in Formula I:

Glucose+O₂ D-glucono-1,5-lactone+H₂O₂ Formula I:

This oxidation reaction produces a current flow. The amplitude of this current is directly related to the concentration of blood glucose. FIG. 3 provides a schematic drawing of the sequences of events that occur in glucose biosensor system. Glucose oxidation by GOx result in D-glucono-δ-lactone. H₂O₂ reduction at Prussian Blue (PB) film is measured by electrons transferred from the working electrode.

In one embodiment, the reagents utilized in the test strip are storage-stable. In certain embodiments, the reagents for use with the test strip are freeze-dried to extend the shelf-life test strip.

The test strip typically includes layers of conductive and non-conductive constituents disposed upon each other to produce a sensor structure.

In one embodiment, the test strip comprises abase substrate; a conductive layer; an insulating layer, a reagent layer; an adhesive layer; a hydrophilic (e.g., nitrocellulose) membrane to which the first binding agent (e.g., aptamer) is attached to capture the target analyte (e.g., antigen); a freeze-dried detectably labeled second binding agent (e.g., Ab-GOx) and glucose; and a top layer.

In another embodiment, the test strip comprises a base substrate; a conductive layer; an insulating layer; a reagent layer; an adhesive layer; and a hydrophilic (e.g., nitrocellulose) membrane to which the first binding agent (e.g., aptamer) is attached to capture the target analyte (e.g., antigen); and freeze-dried glucose and/or the labeled second binding agent (e.g., Ab-GOx) is added to the biological sample containing the target analyte.

The base substrate serves as a matrix for the plurality of constituents that are stacked on top of one another and comprise the functioning sensor. This base constituent can be made of a wide variety of materials having desirable qualities such as dielectric properties, water impermeability, air impermeable, and hermeticity. Some materials include metallic, and/or ceramic and/or polymeric substrates or the like.

The conductive layer is disposed upon the base substrate, wherein the conductive layer that includes at least one electrode (e.g., one, two, or three electrodes) comprising a conductive material for contacting an analyte or its byproduct (e.g. oxygen and/or hydrogen peroxide) to be assayed. The one or more electrodes may include one or more working electrodes and one or more counter, reference, and/or counter/reference electrodes.

The electrodes may be screen-printed electrodes, e.g., screen printed using conductive carbon inks. The materials used may vary. Conductive ink compositions useful for the glucose sensor system described herein include, but are not limited to a silver, carbon, or blended conductive ink. Examples of inks useful to print the working electrode include, but are not limited to, carbon, platinum, carbon/platinum, carbon nanotubes, or other conductive material suitable for the detection of peroxide in the sample.

The electrodes used and the sensitivity required generally dictates the enzyme chemistry that can be employed. For example, a second binding agent linked to glucose oxidase requires excess glucose to detect an analyte in the sample.

A “working electrode” is an electrode at which analyte is electro-oxidized or electro-reduced with or without the agency of a redox mediator. The working electrode can measure an increase or decrease in current in response to exposure to stimuli such as the change in the concentration of the target analyte or molecule or its byproduct. The electrodes provide a detectable signal in the presence of variable concentrations of molecules such as hydrogen peroxide or oxygen.

In addition to the working electrode, the conductive layer may also include a reference electrode (RE) or a combined reference and counter electrode (also termed a quasi-reference electrode or a counter/reference electrode).

In one embodiment, the electrode providing a minimum sensitivity of at least about 50 micromolar glucose concentration and a noise level of less than about 10 nA/mm².

In one embodiment, the insulating layer may be a thin film of insulative (e.g., electrically insulative or water impermeable) material including poly(vinyl chloride), polyethylene, polypropylene, aromatic and aliphatic polyurethenes, aromatic and aliphatic polyurethanes, poly(butylene terephthalate), polybutadiene, silicone rubbers, thiol-ene copolymers, or poly(ethylene-co-vinyl acetate).

In one embodiment, the reagent layer contains mediator for ease of exchange of electrons. In one embodiment, the reagent layer includes a binder; silica; ferricyanide; ferricyanide; 1, 10-phenanthroline Quinone; or an osmium-based mediator.

In one embodiment, the adhesive layer may be an acrylic copolymer, including poly(ethyl acrylate), poly(cyanoacrylate), poly(butyl acrylate), poly(2-ethylhexyl acrylate), or urethane acrylate copolymers.

In one embodiment, the hydrophilic membrane may be comprised of an anionic hydrophilic copolymer, including nitrocellulose, sulfonated tetrafluoroethylene, poly(acrylic acid), or poly(2-acrylamido-2-methyl-1-propanesulfonic acid) (polyAMPS). The membrane may be coated with streptavidin-NC and a first binding agent (e.g., a biotinylated aptamer), which is attached thereto to serve as a capture agent for the target analyte or molecule. Streptavidin-NC is a streptavidin engineered to bind to nitrocellulose.

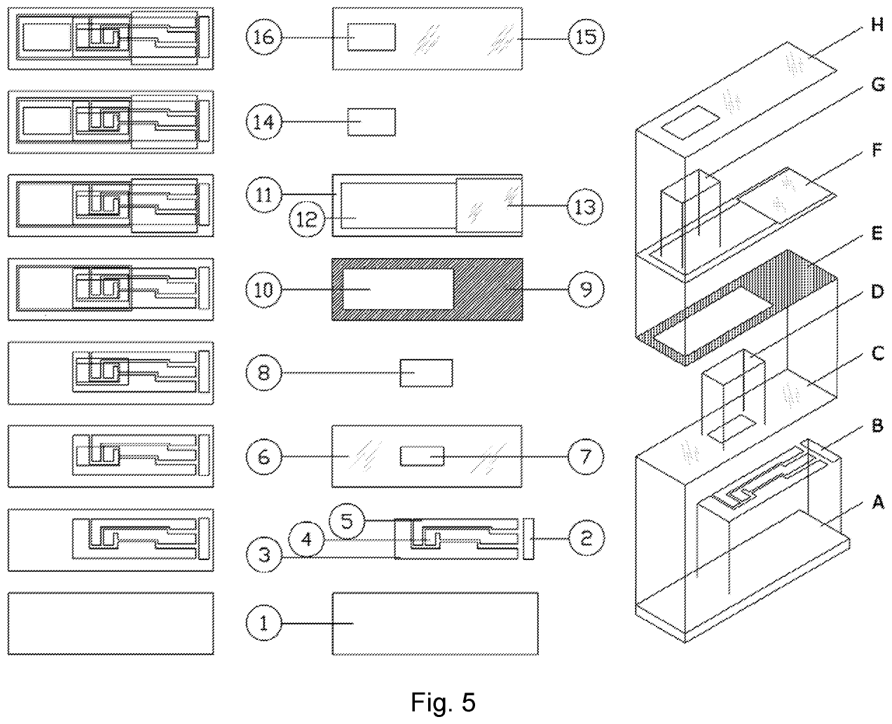

In a particular embodiment, the test strip includes (A) base substrate; (B) a conductive layer which includes three electrodes; (C) insulating layer exposing only part of the electrode the sample to be tested is dropped; (D) reagent layer containing mediator for ease of exchange of electrons; (E) adhesive layer; (F) hydrophilic nitrocellulose membrane, with the proximal membrane containing first binding agent (e.g., aptamer) to capture the target analyte (e.g., antigen) and freeze dried glucose, and the distal end is the paper sink (13); (G) freeze-dried Ab-GOx; and (H) top layer. See e.g., FIG. 5 and labels (A)-(H) and (1)-(16) therein.

In a particular embodiment, the test strip includes (A) base substrate; (B) a conductive layer which includes two electrodes; (C) insulating layer exposing only part of the electrode where the sample to be tested is dropped; (D) reagent layer containing mediator for ease of exchange of electrons; (E) adhesive layer; (F) hydrophilic nitrocellulose membrane, with the proximal membrane containing first binding agent (e.g., aptamer) to capture the target analyte (e.g., antigen) and freeze dried glucose, and the distal end is the paper sink (13); (G) freeze-dried Ab-GOx; and (H) top layer.

According to this embodiment, the base substrate is polyester and an acrylic coating is applied to improve the ink adhesion. Using a CAD (computer-aided design) model of electrode mask, the mask is laser cut onto the base substrate. The electrodes are then screen printed using conductive carbon inks (ERCON INC) followed by an insulation layer (ERCON INC, INSULAYER INK). The two working electrodes can have a surface area of 0.6 mm² each, and the reference electrode can have a surface area of 1.2 mm². In one embodiment, the reagent layer is the mediator layer and consists of a binder, silica, and ferricyanide. This reagent layer is screen printed for two cycles over the working electrodes. In one embodiment, the adhesive layer on top is an acrylic copolymer, the hydrophilic membrane is a nitrocellulose membrane with streptavidin-NC, and biotinylated aptamer is bound (e.g., to streptavidin) to capture the antigen (e.g., a viral antigen). In one embodiment, the top layer is PET (Polyethylene Terephthalate), with a small clear portion to see the sample movement on the strip. The overall dimensions are similar as described for other test strips, e.g., to ensure compatibility with a glucometer, such as LIFESCAN's reader; the test strips can be altered to be compatible with other commercial glucometers.

In one embodiment, before the addition of freeze-dried biological reagents and aptamer immobilization, dropcast GOx is directly dropcast onto the working electrode.

In certain embodiments, the test strip is pre-blocked in order to reduce or eliminate non-specific binding by any suitable blocking agent. Non limiting examples of coating or blocking materials are protein, acryl amide, synthetic polymer, or polysaccharides. In one embodiment, BSA is utilized as a blocking agent. In another embodiment, milk protein, TWEEN, or other surfactant is utilized as a blocking agent.

In certain embodiments, the system permits a low signal to noise ratio, e.g., limits transient non-glucose related signal noise. The composition of the base layer, the method used for depositing the electrodes, the electrode configuration, the electrode materials, the enzyme chemistry used, and other design factors all contribute to the noise of the system.

In one embodiment, the strip has a shelf life of more than 1 year or more than two years, or more than three years.

In some embodiments, the detection of the analyte is performed between 5° C. and 30° C. In one embodiment, the detection of the analyte is performed between 17° C. and 25° C. In one embodiment, the detection of the analyte is performed at a temperature of at least 5° C., at least 6° C., at least 7° C., at least 8° C., at least 9° C., at least 10° C., at least 11° C., at least 12° C., at least 13° C., at least 14° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., or at least 30° C. or more.

In another aspect, described herein is a test strip linked to a detection nucleic acid, e.g., using any linking technique known in the art (e.g., UV cross-linking, or using an affinity pair (e.g., streptavidin and biotin) with one member linked to the detection nucleic acid and the second member linked to the test strip). The detection nucleic can be linked to the test strip on its 3′ end or its 5′ end. In some embodiments of any of the aspects, the detection nucleic acid is linked to a hydrophilic nitrocellulose membrane (e.g., layer F in FIG. 5). In some embodiments of any of the aspects, the detection nucleic acid is linked to a proximal membrane of the test strip (e.g., layer F in FIG. 5). In some embodiments of any of the aspects, the sequence-specific endonuclease, guide nucleic acid, and/or collateral nucleic acid are located in the test strip, but not necessarily linked to the test strip (e.g., in layer G in FIG. 5; e.g., as freeze-dried reagents).

Also disclosed herein are methods of detecting, and optionally monitoring over time, the presence of at least one target analyte, and more particularly at least one target analyte, e.g., a pathogen or component thereof (e.g., a viral antigen) using the system described herein.

In one embodiment, the method comprises: (i) providing a biological sample from a subject, wherein the biological sample is not blood; (ii) adding the biological sample to a test strip, wherein the test strip contains a first and second binding agent capable of creating a detectable complex with at least one target analyte in the biological sample, if present; (iii) introducing the test strip into a glucometer; (iv) incubating the biological sample with the test strip; (v) detecting the level of detectable complex, if any, in the form of hydrogen peroxide generated from glucose oxidation of excess glucose present; and (vi) correlating the level of the detectable complex, if produced, with the quantity of the target analyte in the at least one biological sample, if any, thereby providing a diagnostic assessment.

In one embodiment, the method comprises: (i) collecting a biological sample from a subject, wherein the biological sample is urine, sweat, ocular fluid including aqueous humor, blood, fecal matter, sebum, respiratory droplets, semen, vaginal mucus, cerumen, epidermal cells, nasal sample, cerebral spinal fluid, pleural effusion, or nasopharyngeal specimens; (ii) adding the biological sample to a test strip, wherein the test strip contains a first and second binding agent capable of creating a detectable complex with at least one target analyte in the biological sample, if present; (iii) introducing the test strip into a glucometer; (iv) incubating the biological sample with the test strip; (v) detecting the level of detectable complex, if any, in the form of hydrogen peroxide generated from glucose oxidation of excess glucose present; and (vi) correlating the level of the detectable complex, if produced, with the quantity of the target analyte in the at least one biological sample, if any, thereby providing a diagnostic assessment.

In one embodiment, the method comprises: (i) collecting a biological sample from a subject, wherein the biological sample is not blood, in a tube which dilutes the biological sample by 1× to 1,000,000,000× and contains the second binding agent; (ii) adding the biological sample to a test strip, wherein the test strip contains the first binding agent capable of creating a detectable complex with at least one target analyte in the biological sample, if present; (iii) introducing the test strip into a glucometer; (iv) incubating the biological sample with the test strip; (v) detecting the level of detectable complex, if any, in the form of hydrogen peroxide generated from glucose oxidation of excess glucose present; and (vi) correlating the level of the detectable complex, if produced, with the quantity of the target analyte in the at least one biological sample, if any, thereby providing a diagnostic assessment.

In one embodiment, the method comprises: (i) collecting a biological sample from a subject, wherein the biological sample is urine, sweat, ocular fluid including aqueous humor, blood, fecal matter, sebum, respiratory droplets, semen, vaginal mucus, cerumen, epidermal cells, or nasopharyngeal specimens, in a tube which dilutes the biological sample by 1× to 1,000,000,000× and contains the second binding agent; (ii) adding the biological sample to a test strip, wherein the test strip contains the first binding agent capable of creating a detectable complex with at least one target analyte in the biological sample, if present; (iii) introducing the test strip into a glucometer; (iv) incubating the biological sample with the test strip; (v) detecting the level of detectable complex, if any, in the form of hydrogen peroxide generated from glucose oxidation of excess glucose present; and (vi) correlating the level of the detectable complex, if produced, with the quantity of the target analyte in the at least one biological sample, if any, thereby providing a diagnostic assessment.

In one embodiment, the biological sample is diluted at least 1×, at least 2×, at least 3×, at least 4×, at least 5×, at least 6×, at least 7×, at least 8×, at least 9×, at least 10×, at least 10²×, at least 10³×, at least 10⁴×, at least 10⁵×, at least 10⁶×, at least 10⁷×, at least 10⁸×, or at least 10⁸×, or more, e.g., by a diluent in the sample collection tube. In one embodiment, the biological sample (e.g., saliva) is diluted at least 10×, e.g., by a diluent in the sample collection tube. In one embodiment the diluent is DMEM, PBS, PBST, or another buffer or cell culture medium. The dilution can be performed to reduce non-specific interactions from the different proteins present in the biological sample (e.g., saliva).

In some embodiments, the method further comprises a wash step, e.g., to remove any excess reagents such excess binding agent (e.g., first or second binding agent; e.g., glucose oxidase linked to an aptamer or glucose oxidase linked to an antibody). In some embodiments, the wash step is performed before introducing the test strip into the glucometer. In some embodiments, the wash step can be performed with a diluent (e.g., DMEM, PBS, PBST, or another buffer or cell culture medium) or blocking agent (e.g., BSA, milk protein, TWEEN, or other surfactant) as described herein. In some embodiments, the method does not comprise a wash step.

In one embodiment, the method comprises obtaining multiple test results for the same user taken at different times and comparing these to monitor or predict the likely development of a disease or condition (e.g., COVID-19). In a particular embodiment, the method comprising obtaining at least two, at least three, at least four, or at least five or more test results.

In certain embodiments, the one or more results of the method may be continuously or periodically communicated to a remote entity to determine whether the one or more results are above a threshold level or cut point.

In certain embodiments, the results may be compared to a predetermined reference level. The pre-determined level may be obtained from the general population or from a selected population of subjects. For example, the selected population may be comprised of apparently healthy patients, such as individuals who have not previously had any sign or symptoms indicating the presence of a disease, e.g., an infection. A “predetermined reference level” may be determined, for example, by determining the expression level of the target analyte in a corresponding biological sample obtained from one or more control subject(s) (e.g., not suffering from infection or known not to be susceptible to such a disease). When such a predetermined reference level is used, a higher or increased level determined in a biological sample (i.e., a test sample obtained from the subject) compared to a predetermined reference level is indicative for example that said patient is at risk of developing the disease or has the disease (e.g., COVID-19 infection).

In some embodiments, the method may further comprise the step of recommending instructions for a treatment and/or administering a treatment. In one embodiment, the method comprises identifying that the subject has a level of target analyte above a threshold or cut off level and determining that the subject is therefore a candidate for prophylaxis and/or treatment, e.g., of an infection or pathological condition. The step of “determining” encompasses detecting or quantifying, wherein “detecting” means determining if the target analyte is present or not in the biological sample and “quantifying” means determining the amount of the target analyte present in the biological sample.

The method of the invention may have therapeutic uses for example it may be used for the detection of various pathological conditions or may be used for monitoring the disease stage of a subject or its response to therapy.

In certain embodiments, the method may further comprise using statistical methods to predict the potential for detection of a target analyte to result in disease or progression of disease and/or to permit prognosis of disease (i.e., prediction of the course of a disease).

In certain embodiments, the method may be carried out across a group of population of patients, e.g., in order to permit stratifying the approach to treatment thereof or to satisfy a public health or other monitoring goal.

In one embodiment, a method is disclosed for monitoring the efficiency of a therapeutic regimen in a subject suffering from a pathological condition comprising using the methods and/or system disclosed herein, wherein said target molecule is an antigen associated with the pathological condition, and wherein the amount of said detectable moiety is indicative of the level of the pathological condition and thereby of the efficiency of the therapeutic regimen in the subject.

In certain embodiments, the method comprises monitoring the effectiveness of one more therapeutic agents (e.g., anti-viral agents) over a period of time (e.g., days, weeks) and permits the user to seek an alternative therapeutic approach if the therapeutic agent is not sufficiently effective over a period of time.

In one embodiment, if the treatment regime does not produce a reduction in viral count within a defined period (e.g., days), the user may discontinue the treatment regime in favor of an alternative treatment regime or in certain embodiments, supplement the treatment regime with a second treatment regime. In one embodiment, the system permits obtaining two or more results, three or more results, or five or more results with respect to the quantity of a target analyte for the same user at different times, to permit monitoring of a trend in analyte level over time.

The therapeutic agent may vary. In one embodiment, the therapeutic agent is an anti-viral agent such as a small molecule or biologic anti-viral agent. In certain embodiments, the therapeutic agent is an anti-SARS-CoV-2 agent or an anti-influenza agent.

In another aspect, described herein is a method for detecting a target nucleic acid using a nucleic acid detection system as described herein, comprising: (i) collecting a biological sample from a subject, and optionally, extracting nucleic acid from the biological sample; (ii) contacting the biological sample with a sequence-specific endonuclease, guide nucleic acid, and a collateral nucleic acid, wherein such contacting results in cleavage of the collateral nucleic acid, if the target nucleic acid is present; (iii) adding the biological sample to a test strip in the presence of glucose, wherein the test strip comprises a detection nucleic acid that is capable of creating a detectable complex with the cleaved collateral nucleic acid, if present; (iv) incubating or not incubating the biological sample with the test strip; (v) introducing the test strip into a detection device; (vi) detecting the level of detectable complex, if any, through a chemical reaction between glucose and glucose oxidase; and (vii) correlating the level of the detectable complex, if produced, with the quantity of the target analyte in the at least one biological sample, thereby providing a diagnostic assessment.

In some embodiments of any of the aspects, the biological sample is saliva. In some embodiments of any of the aspects, the biological sample is not blood. In some embodiments of any of the aspects, the detection device is a glucose meter.

In some embodiments of any of the aspects, the method further comprises (viii) transmitting the diagnostic assessment or result to an electronic device, data base, or cloud server for subsequent review by a clinician or trained healthcare provider; and (ix) providing the diagnostic assessment to the individual who performed the method of diagnostic assessment. In some embodiments of any of the aspects, the individual is the subject. In some embodiments of any of the aspects, the method further comprises (viii) recommending, instructing and/or administering one or more therapeutic regimes to the subject in response to the diagnostic assessment.

Also disclosed are methods for manufacturing the systems and tests strips disclosed herein.

The test strip can be manufactured using any suitable method. In one embodiment, the test trip is manufactured using a roll to roll process, a screen-printing process, a drop-cast process, or combinations thereof. Exemplary methods of manufacture are provided in the Examples.

Test strips compositions as disclosed herein may be combined with other ingredients or reagents or prepared as components of kits or other retail products for commercial sale or distribution. The kit can also contain instructions or informational material regarding administration and/or use of the kit. The kit may also contain a reader or detection device as described herein.

In one embodiment, the kit comprises a glucose meter, at least one test strip as described herein, and a container or pouch for storing the at least one test strip during transport. In some embodiments, the kit comprises a test strip that comprises a first and second binding agent capable of creating a detectable complex with the at least one target analyte (e.g., an aptamer and an antibody, or two antibodies, or two aptamers). In some embodiments, the kit comprises a test strip that comprises a detection nucleic acid (e.g., linked to the test strip); the test strip in the kit can further comprise a sequence-specific endonuclease, guide nucleic acid, and/or collateral nucleic acid, or the sequence-specific endonuclease, guide nucleic acid, and/or collateral nucleic acid can be provided separately in the kit from the test strip.

In some embodiments, the kit comprises an effective amount of glucose. In some embodiments, the kit comprises an effective amount of a sequence-specific endonuclease, a guide nucleic acid, and a collateral nucleic acid, and the test strip comprises a detection nucleic acid.

In some embodiments, the components described herein can be provided singularly or in any combination as a kit. Such kits can optionally include one or more agents that permit the detection of the detectable complexes described herein.

In some embodiments, the compositions in the kit can be provided in a watertight or gas tight container which in some embodiments is substantially free of other components of the kit. For example, the test strips can be supplied in more than one container, e.g., it can be supplied in a container having sufficient reagent for a predetermined number of detection reactions, e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in its form. In one embodiment, the informational material can include information about production of the test strips, concentration, date of expiration, batch or production site information, and so forth. In one embodiment, the informational material relates to methods for using or administering the components of the kit.

The kit can include a component for the detection of a target analyte. In addition, the kit can include one or more aptamers or antibodies that a target analyte. The aptamers or antibodies can be supplied in dry preparation, e.g., lyophilized, or in a solution. The antibody or other detection reagent can be linked to a label, e.g., a radiological, fluorescent (e.g., GFP) or colorimetric label for use in detection.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A system for detecting at least one target analyte in a biological sample, wherein the system comprises (i) a two binding agent assay, wherein the assay contains a first and second binding agent capable of creating a detectable complex with the at least one target analyte; and (ii) a detection device for detecting the detectable complex, wherein the detection device is an oxidase-based amperometric sensor and the biological sample is present in sweat, saliva, serum, mucus, or blood.
2. The system of paragraph 1, wherein the amperometric sensor is an oxidase-based amperometric sensor.
3. The system of paragraph 2, wherein the amperometric sensor is a hydrogenase-or dehydrogenase-based amperometric.
4. The system of any of the preceding paragraphs, wherein the biological sample is sweat, saliva, serum, mucus, or blood.
5. The system of any of the preceding paragraphs, wherein the biological sample is saliva.
6. The system of any of the preceding paragraphs, wherein the detection device is a glucose meter.
7. The system of paragraph 6, wherein the glucose meter comprises a glucose sensor having a sensor output related to glucose in a biological sample on a test strip.
8. The system of any of the preceding paragraphs, wherein the biological sample is mixed with a sugar such as but not limited to glucose.
9. The system of paragraph 8, wherein biological sample is mixed with a sugar such as but not limited to glucose at a concentration between about 0.01 mM and about 1 M.
10. The system of any of the preceding paragraphs, wherein the first and second binding agents are selected from the group consisting of aptamers, antibodies, proteins, or a combination thereof.
11. The system of any of the preceding paragraphs, wherein the first binding agent is an aptamer and the second binding agent is an antibody, wherein the antibody is linked to glucose oxidase.
12. The system of any of the preceding paragraphs, wherein the first and second binding agents bind to different sites on the target analyte.
13. The system of paragraph 12, wherein the first and second binding agents have a Kd for the target analyte from between about 1:1000 to about 1000:1.
14. The system of paragraph 12 or 13, wherein the binding affinity of the first binding agent is weaker than the binding affinity of the second binding agent.
15. The system of any of the preceding paragraphs, wherein the target analyte is a whole virus or component thereof.
16. The system of paragraph 15, wherein the virus is a betacoronavirus, an influenza virus, an HIV virus, or hepatitis virus.
17. The system of paragraph 16, wherein the target coronavirus analyte is SARS-CoV-2 or a component thereof.
18. The system of paragraph 17, wherein the target analyte is a component of SARS-CoV-2 selected from the group consisting of the spike protein, the membrane protein, the hemagglutinin protein, or the envelope protein.
19. The system of any of paragraphs 1-14, wherein the target analyte is selected from the group consisting of IgG, IgM, and IgA.
20. A system for detecting at least one virus in a biological sample, wherein the system comprises: (i) a two binding agent assay, wherein the assay contains a first and second binding agent capable of creating a detectable complex with the at least one virus; and (ii) a detection device for detecting the detectable complex, wherein the detection device is a glucose meter.
21. The system of paragraph 20, wherein the glucose meter comprises a glucose sensor having a sensor output related to glucose in a biological sample on a test strip.
22. The system of paragraph 20, wherein the biological sample is mixed with a sugar such as but not limited to glucose.
23. The system of paragraph 22, wherein biological sample is mixed with a sugar such as but not limited to glucose at a concentration between about 0.01 mM and about 1 M.
24. The system of any of paragraphs 20-23, wherein the first and second binding agents are selected from the group consisting of aptamers, antibodies, proteins, or a combination thereof.
25. The system of any of paragraphs 20-24, wherein the first binding agent is an aptamer, and the second binding agent is an antibody, wherein the antibody is linked to glucose oxidase.
26. The system of any of paragraphs 20-25, wherein the first and second binding agents bind to different sites on the target analyte.
27. The system of paragraph 26, wherein the first and second binding agents have a Kd for the target analyte from between about 1:1000 to about 1000:1.
28. The system of paragraph 26 or 27, wherein the binding affinity of the first binding agent is weaker than the binding affinity of the second binding agent.
29. The system of any of paragraphs 20-28, wherein the target analyte is a whole virus or component thereof.
30. The system of paragraph 29, wherein the virus is a betacoronavirus.
31. The system of paragraph 30, wherein the target analyte is SARS-CoV-2 or a component thereof.
32. The system of paragraph 31, wherein the target analyte is a component of SARS-CoV-2 selected from the group consisting of the spike protein, the membrane protein, the hemagglutinin protein, or the envelope protein.
33. The system of any of paragraphs 20-28, wherein the target analyte is selected from the group consisting of IgG, IgM, and IgA.
34. A method for a diagnostic assessment, comprising: (i) collecting a biological sample from a subject, wherein the biological sample is not blood; (ii) adding the biological sample to a test strip in the presence of glucose, wherein the test strip contains a first and second binding agent capable of creating a detectable complex with at least one target analyte, if present in the biological sample; (iii) incubating or not incubating the biological sample with the test strip; (iv) introducing the test strip into a detection device; (v) detecting the level of detectable complex, if any, through a chemical reaction between glucose and glucose oxidase; and (vi) correlating the level of the detectable complex, if produced, with the quantity of the target analyte in the at least one biological sample, if any, thereby providing a diagnostic assessment.
35. A method for diagnostic assessment, comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) diluting the collected sample by 1×-100× in an aqueous solution/mixture in the presence of second binding agent; (iii) adding the biological sample and second binding agent to a test strip, wherein the test strip contains a first binding agent capable of creating a detectable complex with at least one target analyte in the biological sample, if present; (iv) incubating or not incubating the biological sample with the test strip; (v) introducing the test strip into a detection device; (vi) detecting the level of detectable complex, if any, through a chemical reaction between glucose and glucose oxidase; and (vii) correlating the level of the detectable complex, if produced, with the quantity of the target analyte in the at least one biological sample, if any, thereby providing a diagnostic assessment.
36. The method of paragraphs 34 and 35, wherein the biological sample is saliva.
37. The method of paragraphs 34 and 35, wherein the detection device is a glucose meter.
38. The method of paragraphs 34 and 35, further comprising: (viii) transmitting the diagnostic assessment or result to an electronic device, data base, or cloud server for subsequent review by a clinician or trained healthcare provider; and (ix) providing the diagnostic assessment to the individual who performed the method of diagnostic assessment.
39. The method of paragraph 38, wherein the individual is the subject.
40. The method of any of paragraphs 34-37, further comprising (vi, viii) recommending, instructing and/or administering one or more therapeutic regimes to the subject in response to the diagnostic assessment.
41. A method for a diagnostic assessment, comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) adding the biological sample to a test strip, wherein the test strip contains a first and second binding agent capable of creating a detectable complex with at least one target analyte in the biological sample, if present; (iii) incubating or not incubating the biological sample with the test strip; (iv) introducing the test strip into a detection device such as a glucometer; (v) detecting the level of detectable complex, if any, through a chemical reaction between glucose and glucose oxidase; and (vi) correlating the level of the detectable complex, if produced, with the quantity of the target analyte in the at least one biological sample, thereby providing a diagnostic assessment.
42. A method for a diagnostic assessment, comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) diluting the collected sample by 1×-100× in an aqueous solution/mixture in the presence of second binding agent; (iii) adding the biological sample and second binding agent to a test strip, wherein the test strip contains a first binding agent capable of creating a detectable complex with at least one target analyte in the biological sample, if present; (iv) incubating or not incubating the biological sample with the test strip; (v) introducing the test strip into a detection device such as a glucometer; (vi) detecting the level of detectable complex, if any, through a chemical reaction between glucose and glucose oxidase; and (vii) correlating the level of the detectable complex, if produced, with the quantity of the target analyte in the at least one biological sample, thereby providing a diagnostic assessment.
43. The method of paragraphs 41 and 42, further comprising (viii) recommending, instructing and/or administering one or more therapeutic regimes in response to the diagnostic assessment.
44. A method for a diagnostic assessment, comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) adding the biological sample to a test strip, wherein the test strip contains a first and second binding agent capable of creating a detectable complex with at least one target analyte in the biological sample, if present; (iii) incubating the biological sample with the test strip; (iv) introducing the test strip into a detection device such as a glucometer; (v) detecting the level of detectable complex, if any, through a chemical reaction between glucose and glucose oxidase; (vi) correlating the level of the detectable complex, if produced, with the quantity of the target analyte in the at least one biological sample, if any, thereby providing a diagnostic assessment; (vii) transmitting the diagnostic assessment or result to an electronic device, data base, or cloud server for subsequent review by a clinician or trained healthcare provider; and (viii) transmission of the diagnostic assessment or outcome to the individual who performed the method of diagnostic assessment.
45. A method for a diagnostic assessment, comprising: (i) collecting a biological sample from a subject, wherein the biological sample is from urine, sweat, ocular fluid including aqueous humor, blood, fecal matter, sebum, respiratory droplets, semen, vaginal mucus, cerumen, epidermal cells, or nasopharyngeal specimens; (ii) diluting the collected sample by 1×-100× in an aqueous solution/mixture in the presence of second binding agent; (iii) adding the biological sample to a test strip, wherein the test strip contains a first binding agent capable of creating a detectable complex with at least one target analyte in the biological sample, if present; (iv) incubating the biological sample with the test strip; (v) introducing the test strip into a detection device such as a glucometer; (vi) detecting the level of detectable complex, if any, through a chemical reaction between glucose and glucose oxidase; (vii) correlating the level of the detectable complex, if produced, with the quantity of the target analyte in the at least one biological sample, if any, thereby providing a diagnostic assessment; (viii) transmitting the diagnostic assessment or result to an electronic device, data base, or cloud server for subsequent review by a clinician or trained healthcare provider; and (ix) transmission of the diagnostic assessment or outcome to the individual who performed the method of diagnostic assessment.
46. The method of paragraphs 44 and 45, wherein the individual is the subject.
47. The method of paragraphs 44 and 45, further comprising (ix, x) recommending, instructing and/or administering one or more therapeutic regimes to the subject in response to the diagnostic assessment.
48. The system of any one of paragraphs 1-33 or 76-98, or the methods of any one of paragraphs 34-47 or 56-75 or 99-104 wherein one or more different target analytes are assessed at the same time or sequentially.
49. A test strip for use in the systems of any one of paragraphs 1-33, or the methods of any one of paragraphs 34-47 or 56-75, wherein the test strip comprises at least one of the following: (i) a substrate, at least one first and second binding agent and two or more electrodes; (ii) the substrate both first and second binding agents and two or more electrodes; (iii) at least one first and second binding agent and two or more electrodes; or (iv) both first and second binding agents and two or more electrodes.
50. A test trip comprising at least one of the following: (i) one or more planar or co-planar electrode made of carbon, iron, palladium, platinum or gold; (ii) an electrode coated with iron salts such as ferrous ferrocyanide salts, as a mediator; (iii) an electrode coated with Prussian blue as a mediator; (iv) an n-electrode set-up where an electrode is a (semi-) conductive solid that interfaces with an electrolyte solution; (v) the electrode set-up includes a working electrode, a reference electrode, and a counter or auxiliary electrode; (vi) a two-electrode set-up that has the current and sense leads connected together, a working and working sense are connected to a working electrode and reference and counter are connected to a second auxiliary, counter, or quasi-/pseudo-reference electrode; (vii) a three electrode set-up, the reference lead is separated from the counter and connected to a third electrode most often positioned so that it is measuring a point very close to the working electrode that has both working and working sense leads attached; (viii) a four-electrode set-up with the working sense lead decoupled from the working electrode, in addition to the reference lead; and/or (ix) a zero resistance ammeter where the working and counter electrode leads are shorted together inside the strip so that there is zero net voltage dropped across the whole electrochemical cell.
51. A test strip of paragraphs 49 or 50 wherein the first binding agent is immobilized on a porous polymer, metal, or ceramic membrane and said membrane is placed above or between the electrodes.
52. A test strip of paragraph 51 wherein said membrane is nitrocellulose.
53. A kit comprising the test strip of any one of paragraphs 49, 50, 51, or 52.
54. The kit of paragraph 53, comprising instructions for using the test strip.
55. The kit of paragraph 53, further comprising a glucometer or other electrochemical detection device.
56. A method comprising: (i) providing a biological sample from a subject, (ii) detecting the presence of a target analyte in the biological sample utilizing the system of paragraphs above, wherein the target analyte is the SARS-CoV-2 virus or a component thereof, and (iii) optionally, treating the subject with a therapeutic agent.
57. The method of paragraph 56, wherein the biologic sample is saliva.
58. The method of any of paragraphs 56-57, wherein the target analyte is the SARS-CoV-2 virus.
59. The method of any of paragraphs 56-57, wherein the target analyte is a component of the SARS-CoV-2 virus.
60. The method of any of paragraphs 56-59, wherein the component is the S protein or a fragment thereof.
61. A method comprising: (i) providing a biological sample from a subject, (ii) detecting the presence of a target analyte in the biological sample utilizing the system of paragraphs above, wherein the target analyte is a CoV virus or a component thereof and (iii) optionally, treating the subject with a therapeutic agent.
62. The method of paragraph 61, wherein the biologic sample is saliva.
63. The method of paragraph 61 or 62, wherein the target analyte is a CoV virus.
64. A method comprising: (i) providing a biological sample from a subject, (ii) detecting the presence of a target analyte in the biological sample utilizing the system of paragraphs above, wherein the target analyte is influenza virus or a component thereof and (iii) optionally, treating the subject with a therapeutic agent.
65. The method of paragraph 64, wherein the biologic sample is saliva.
66. The method of paragraph 64 or 65, wherein the target analyte is an influenza virus.
67. The method of paragraph 64 or 65, wherein the target analyte is a component of an influenza virus.
68. A method comprising: (i) providing a biological sample from a subject, (ii) detecting the presence of a target analyte in the biological sample utilizing the system of paragraphs above, wherein the target analyte is HIV virus or a component thereof and (iii) optionally, treating the subject with a therapeutic agent.
69. The method of paragraph 68, wherein the biologic sample is saliva.
70. The method of paragraph 68 or 69, wherein the target analyte is an HIV virus.
71. The method of paragraph 68 or 69, wherein the target analyte is a component of an HIV virus.
72. A method comprising: (i) providing a biological sample from a subject, (ii) detecting the presence of a target analyte in the biological sample utilizing the system of paragraphs above, wherein the target analyte is a hepatitis virus or a component thereof and (iii) optionally, treating the subject with a therapeutic agent.
73. The method of paragraph 72, wherein the biologic sample is saliva.
74. The method of paragraph 72 or 73, wherein the target analyte is a hepatitis virus.
75. The method of paragraph 72 or 73, wherein the target analyte is a component of a hepatitis virus.
76. A system for detecting at least one target nucleic acid in a biological sample, wherein the system comprises (i) a sequence-specific endonuclease and guide nucleic acid that cleave a collateral nucleic acid upon specific binding of the target nucleic acid to the endonuclease and guide nucleic acid; (ii) a detection nucleic acid that is capable of creating a detectable complex with the cleaved collateral nucleic acid; and (iii) a detection device for detecting the detectable complex, wherein the detection device is an oxidase-based amperometric sensor and the biological sample is present in sweat, salvia, serum, mucus, or blood.
77. The system of paragraph 76, wherein the sequence-specific endonuclease is a Cas enzyme.
78. The system of paragraph 76 or 77, wherein the sequence-specific endonuclease is Casl2a or Cas13.
79. The system of any one of paragraphs 76-78, wherein the guide nucleic acid is complementary or substantially complementary to at least a portion of the target nucleic acid.
80. The system of any one of paragraphs 76-79, wherein the detection nucleic acid is complementary or substantially complementary to at least a portion of the cleaved collateral nucleic acid.
81. The system of any one of paragraphs 76-80, wherein the detection nucleic acid hybridizes to the cleaved collateral nucleic acid.
82. The system of any one of paragraphs 76-81, wherein the detection nucleic acid does not hybridize to the un-cleaved collateral nucleic acid.
83. The system of any one of paragraphs 76-82, wherein the detection nucleic acid is linked to a test strip.
84. The system of any one of paragraphs 76-83, wherein the collateral nucleic acid is linked to glucose oxidase.
85. The system of any one of paragraphs 76-84, wherein the system further comprises an aptamer linked to glucose oxidase.
86. The system of paragraph 85, wherein the aptamer specifically binds to at least a portion of the cleaved collateral nucleic acid.
87. The system of paragraph 86, wherein the aptamer binds to a single-stranded portion of the cleaved collateral nucleic acid.
88. The system of paragraph 86, wherein the aptamer binds to a double-stranded portion of the cleaved collateral nucleic acid hybridized to the detection nucleic acid.
89. The system of any one of paragraphs 76-88, wherein the system further comprises an antibody linked to glucose oxidase.
90. The system of paragraph 89, wherein the antibody specifically binds to at least a portion of the cleaved collateral nucleic acid.
91. The system of any one of paragraphs 76-90, wherein the collateral nucleic acid is linked to an antibody that specifically binds glucose oxidase.
92. The system of any one of paragraphs 76-91, wherein the collateral nucleic acid is linked to a first member of an affinity pair.
93. The system of paragraph 92, further comprising glucose oxidase linked to a second member of an affinity pair.
94. The system of paragraph 92 or 93, wherein the first and second members of the affinity pair is selected from the group consisting of: a haptenic or antigenic compound in combination with a corresponding antibody or binding portion or fragment thereof, digoxigenin and anti-digoxigenin; mouse immunoglobulin and goat anti-mouse immunoglobulin; a non-immunological binding pair; biotin and avidin; biotin and streptavidin; a hormone and a hormone-binding protein; thyroxine and cortisol-hormone binding protein; a receptor and a receptor agonist; a receptor and a receptor antagonist; acetylcholine receptor and acetylcholine or an analog thereof, IgG and protein A; lectin and carbohydrate; an enzyme and an enzyme cofactor; an enzyme and an enzyme inhibitor; complementary oligonucleotide pairs capable of forming nucleic acid duplexes; and a first molecule that is negatively charged and a second molecule that is positively charged.
95. The system of paragraph 92 or 93, wherein the first and second members of the affinity pair is streptavidin and biotin.
96. The system of any one of paragraphs 92-95, wherein streptavidin is linked to the collateral nucleic acid, and biotin is linked to the glucose oxidase.
97. The system of any one of paragraphs 92-95, wherein biotin is linked to the collateral nucleic acid, and streptavidin is linked to the glucose oxidase.
98. The system of any one of paragraphs 76-97, wherein the target nucleic acid is a viral nucleic acid.
99. A method for detecting a target nucleic acid using the system of any one of paragraphs 76-98 comprising:
   (i) collecting a biological sample from a subject, and optionally, extracting nucleic acid from the biological sample;
   (ii) contacting the biological sample with a sequence-specific endonuclease, guide nucleic acid, and a collateral nucleic acid, wherein such contacting results in cleavage of the collateral nucleic acid, if the target nucleic acid is present;
   (iii) adding the biological sample to a test strip in the presence of glucose, wherein the test strip comprises a detection nucleic acid that is capable of creating a detectable complex with the cleaved collateral nucleic acid, if present;
   (iv) incubating or not incubating the biological sample with the test strip;
   (v) introducing the test strip into a detection device;
   (vi) detecting the level of detectable complex, if any, through a chemical reaction between glucose and glucose oxidase; and
   (vii) correlating the level of the detectable complex, if produced, with the quantity of the target analyte in the at least one biological sample, thereby providing a diagnostic assessment.
100. The method of paragraph 99, wherein the biological sample is saliva.
101. The method of paragraphs 99 or 100, wherein the detection device is a glucose meter.
102. The method of any one of paragraphs 99-101, further comprising (viii) transmitting the diagnostic assessment or result to an electronic device, data base, or cloud server for subsequent review by a clinician or trained healthcare provider; and (ix) providing the diagnostic assessment to the individual who performed the method of diagnostic assessment.
103. The method of any one of paragraphs 99-102, wherein the individual is the subject.
104. The method of any one of paragraphs 99-103, further comprising (viii) recommending, instructing and/or administering one or more therapeutic regimes to the subject in response to the diagnostic assessment.
105. The test strip of any one of paragraphs 49-52, linked to a detection nucleic acid.
106. A kit comprising the test strip of paragraph 105.
107. A sensor for detecting at least one target analyte in a biological sample, wherein the sensor comprises (i) a two binding agent assay, wherein the assay contains a first and second binding agent capable of creating a detectable complex with the at least one target analyte; and (ii) a detection device for detecting the detectable complex, wherein the detection device is an oxidase-based amperometric sensor and the biological sample is present in sweat, saliva, serum, mucus, or blood.
108. A sensor for detecting at least one virus in a biological sample, wherein the sensor comprises: (i) a two binding agent assay, wherein the assay contains a first and second binding agent capable of creating a detectable complex with the at least one virus; and (ii) a detection device for detecting the detectable complex, wherein the detection device is a glucose meter.
109. A sensor for detecting at least one target nucleic acid in a biological sample, wherein the sensor comprises (i) a sequence-specific endonuclease and guide nucleic acid that cleave a collateral nucleic acid upon specific binding of the target nucleic acid to the endonuclease and guide nucleic acid; (ii) a detection nucleic acid that is capable of creating a detectable complex with the cleaved collateral nucleic acid; and (iii) a detection device for detecting the detectable complex, wherein the detection device is an oxidase-based amperometric sensor and the biological sample is present in sweat, salvia, serum, mucus, or blood.

### EXAMPLES

### Example 1: Detection of Influenza

Viral culture for influenza. The cell culture and passage were performed according to the WHO's MDCK (Madin-Darby Canine Kidney) cell culture protocol. After the cells reached 70%-80% confluent, the DMEM media (GIBCO Dulbecco's Modified Eagle Medium) was aspirated, and the cells were washed three times in 5 mL of 1×PBS (phosphate-buffered saline). The virion stock of influenza H1N1 A/Puerto Rico/8/34 (A/PR/8/34) was thawed in a 37° C. bath, and 500 μL-1000 μL of virion specimen was inoculated onto a T-75 flask. The flask was slowly tilted and rotated to spread the viral inoculum. The inoculum was allowed to adsorb for 30 min in the 37° C. incubator. 12 mL of viral growth DMEM (containing TPCK-Trypsin) was added into the same T75 flask (tosyl phenylalanyl chloromethyl ketone (TPCK) irreversibly inhibits the serine protease α-chymotrypsin). This was incubated at 37° C. and inspected daily for cytopathic effects (e.g., darkening of flask due to lysis). When the cytopathic effects were around 75%-100%, 12 mL of supernatant was collected using a serological pipette. 15% glycerol was added to the final solution, and aliquots were frozen at −80° C. for storage. The stocks had a concentration of 5×10⁶ PFU/mL (plaque forming units per milliliter) measured via plaque assay and 1.64×10⁸ RNA copies/mL via RT-PCR (reverse transcription polymerase chain reaction) assay.

Design of the sensor. A sandwiched electrochemical detection mechanism was designed and constructed consisting of an aptamer and an antibody labeled with glucose oxidase (GOx), both of which bind the virus of interest. As shown in FIG. 2, the aptamer (bound to the nitrocellulose membrane) captures the viral antigen, and Ab-GOx binds only if viral antigen is present. On application of a constant potential, GOx oxidizes glucose (e.g., 500 mM), transfers an electron to oxygen, produces hydrogen peroxide, and generates a current output via an electrode that reacts with hydrogen peroxide. The electrode of sensor includes a mediator layer (e.g., Prussian blue) to lower the overvoltage of the hydrogen peroxide production generates an output current at a lower potential. Detection at a higher potential can be accomplished without a mediator layer.

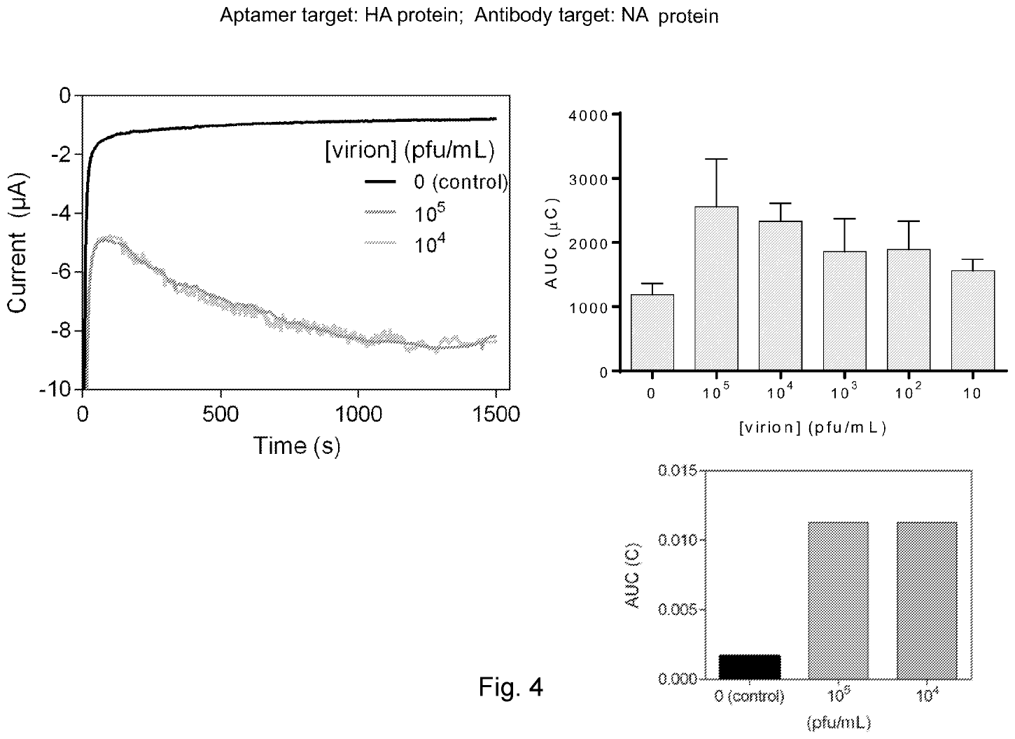

Detection of H1N1 Influenza. For the sandwiched sensing assay, an anti-influenza A HIN1 neuraminidase antibody was used (ABCAM). This antibody has high affinity towards the neuraminidase proteins on the viral membrane. Similarly, an aptamer having affinity towards the hemagglutinin protein was used (IDT TECHNOLOGIES). A nitrocellulose membrane with 0.45 μm pores was used (THERMOSCIENTIFIC). A 4 mm in diameter hole was punched in the membrane. The membrane was functionalized with streptavidin-NC by dropcasting streptavidin (0.5 mg/mL in PBS) onto the membrane. The membrane was dried at 37° C. and stored under dry conditions overnight before use. Next, 10 μL of 20 μM of biotinylated aptamer was dropcast for 30 min followed by washing in PBS buffer. Following aptamer immobilization, the surface was blocked by adding 1% BSA (bovine serum albumin) on the surface, followed by washing. The virion stock from −80° C. was thawed and centrifuged at 3000 rpm to remove cellular debris. Two ten-fold dilutions were performed on the supernatant virion stock to obtain 105 and 10⁴ PFU/mL of virion concentration. 10 uL of these solutions was added dropwise to two different membranes with aptamer and incubated for 15 min. The GOx conjugated antibody, synthesized using ABCAM'S LIGHTNING-LINK (GOx conjugate kit, #ab102887), was diluted in PBST (1 mg/mL GOx conjugated antibody in PBS with the detergent TWEEN 20, e.g., 0.05%) and dropcasted and spread on the membrane surface and incubated for 15 min. Next the membrane was washed using 200 μL PBST and transferred to the DROPSENS 710 electrode and placed above the electrode. The chronoamperometric measurements were performed after addition of 50 uL of 500 mM glucose solution. For the negative control sample, instead of adding the virion stock, a DMEM solution (0.2% BSA, 25 mM HEPES (4-(2-hydroxyethyl)-1-piperazineethanesulfonic acid), 2 μg/mL TPCK-Trypsin) containing no virus was used. Chronoamperometric measurements were performed at a potential of −0.2 V versus Ag/AgCl (e.g., the reference electrode) using a homemade potentiostat, and current was monitored over time. FIG. 4 shows that as the virions were present in the sample, the bound glucose oxidase oxidized glucose and produced a current signal, whereas in the control sample case (no virions were present), negligible or background current was measured. Area under the curve of a current vs. time plot gives the total charge transferred, and FIG. 4 shows that as the virions were present, charge transferred was an order of magnitude higher than in the case of control sample. Thus, a significant current signal was observed in the presence of physiologically relevant viral concentration (10⁴ pfu/mL) and can be replicated for other viruses using the corresponding antibody-aptamer combination.

Calibration plot for detection of H1N1. The sensor was prepared by addition of 10 μL of 1 mg/mL Streptavidin-NC followed by 10 μL of 40 μM of aptamer targeting the hemagglutinin protein of H1N1. Blocking agent was 5 μL of 3% BSA. The antibody-GOx concentration used for this experiment was 0.1 mg/mL in PBST. Different concentrations of virus 10⁵ pfu/mL-10 pfu/mL were prepared by dilutions in DMEM buffer. For the control sample, DMEM buffer with no virus was also evaluated. The virions were incubated for 15 minutes, followed by Ab-GOx addition, followed by a wash with PBST (0.05% TWEEN-20) wash. Chronoamperometric measurements were performed at −0.2 V for 1500 s. The area under the curve of current vs. time graph was plotted for different virion concentrations. There was a statistically significant difference between 10⁵-10² pfu/mL virial concentrations and the control sample (see e.g., FIG. 4).

Detection of HIN1 in human saliva. Human saliva, which tested negative for infectious diseases, was used (LEE BIOSOLUTIONS, Catalog #991-05-S). The saliva samples contain a very high protein concentration. This inhibited the specific antibody-viral binding. Hence the saliva sample was diluted 10 and 100 times in DMEM buffer before adding to the membrane surface. Here an antibody concentration of 1 mg/mL was used. A significant difference of current was observed when the saliva sample was diluted 10 times in comparison to the control sample (i.e., saliva diluted in buffer 10 times). Henceforth, 10-fold saliva dilutions were used for further experiments. The dilution is performed to reduce the non-specific interactions from the different proteins present in the saliva. Similar steps as described above were followed to obtain a current output. The human saliva was spiked with different viral concentrations 10⁴-10² pfu/mL and diluted 10-fold, and the current vs. time was plotted. A significantly higher current was observed in the case of viral samples in saliva than the control sample (i.e., saliva with no virus present diluted 10-fold)

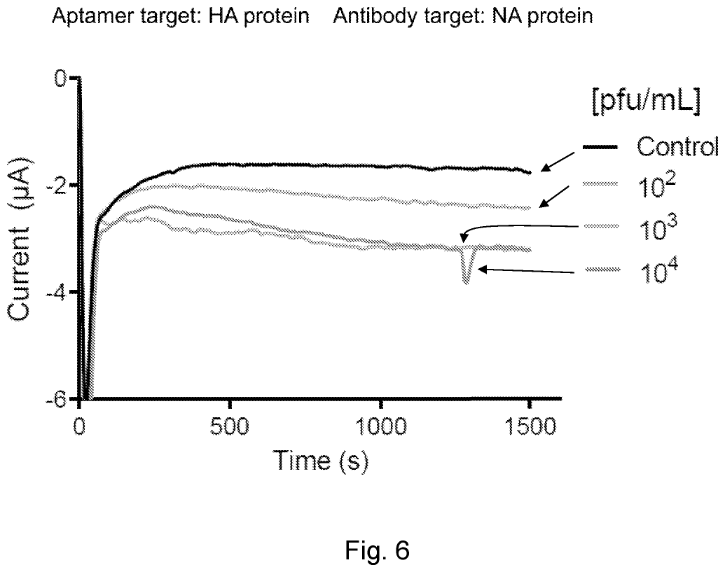

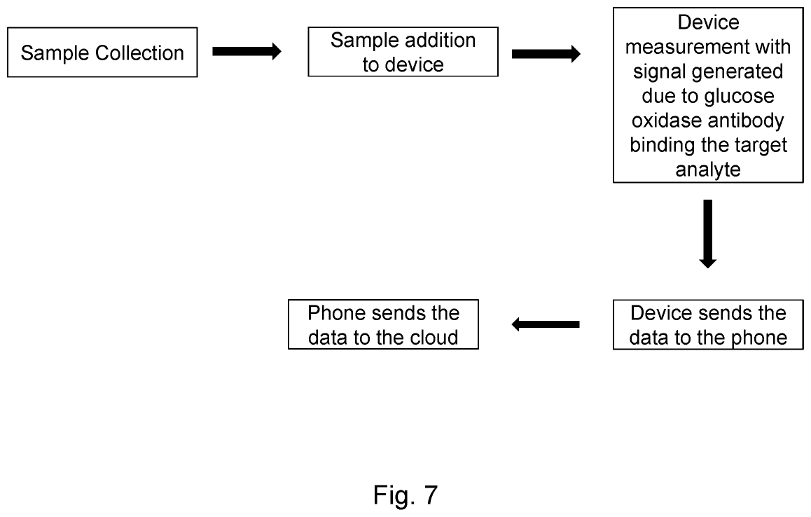

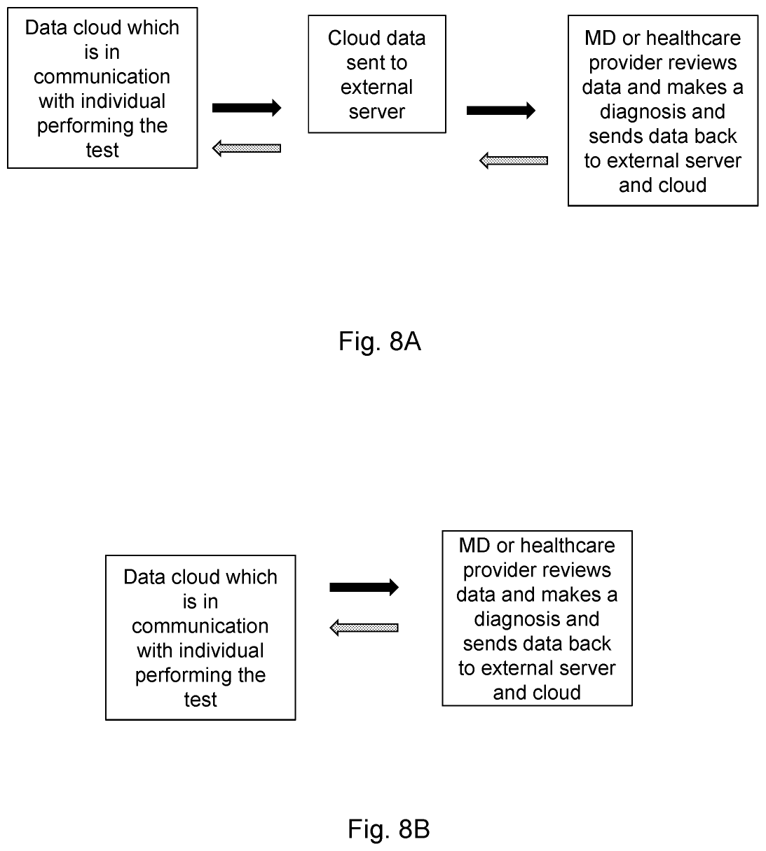

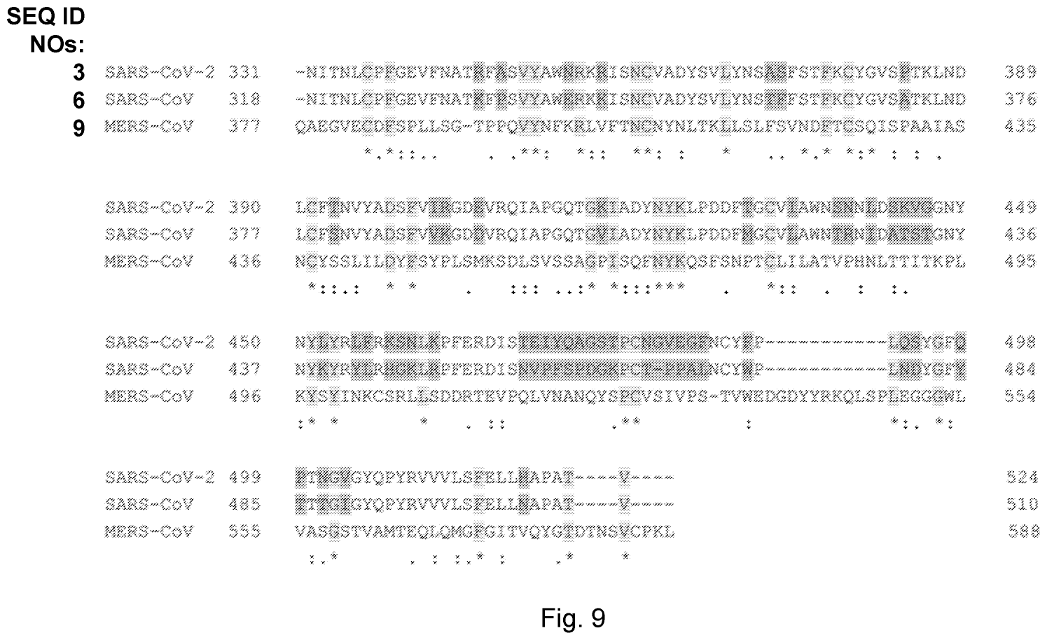

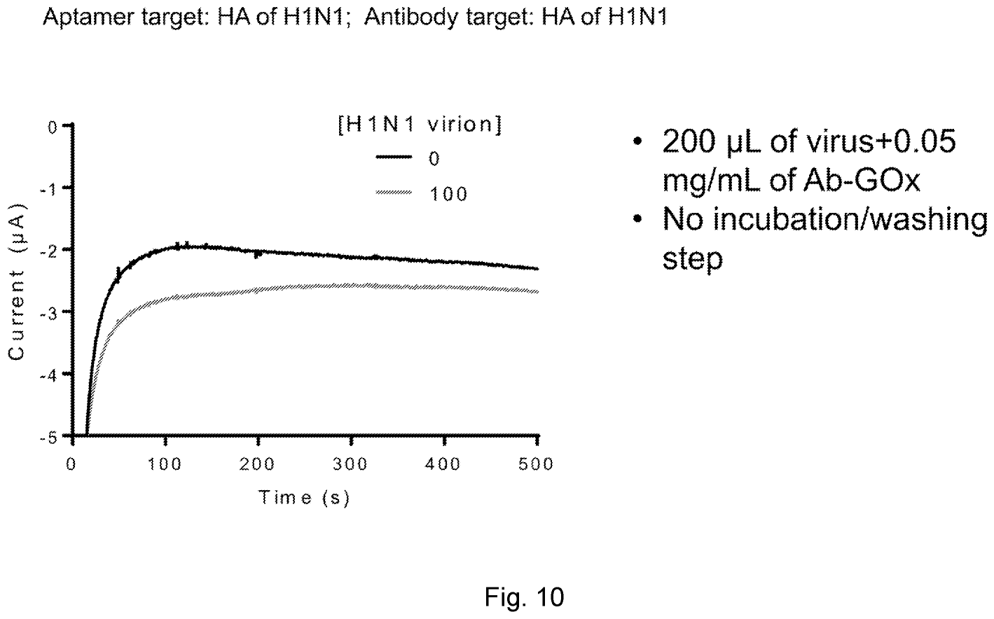

Comparison of different antibody targets for HIN1 detection. Antibodies targeting the hemagglutinin (HA; e.g., anti-HA mouse monoclonal antibody) and neuraminidase (NA; e.g., anti-NA rabbit polyclonal antibodies) protein on the HIN1 viral membrane surface were also tested. The antibodies used were ab 128412 (anti-Influenza A HIN1 hemagglutinin antibody [C102 (IV.C102)], ABCAM) and ab 91646 (anti-Influenza A HIN1 neuraminidase antibody, ABCAM). A higher current signal was observed in the case of NA-targeting antibody as the aptamer and an antibody targeting different proteins (e.g., HA) on the viral membrane surface; alternatively, the aptamer target can be HA protein, and the antibody target can be NA protein (see e.g., FIG. 6). In case of targeting the same protein (e.g., HA) by both the aptamer and antibody, the binding affinities of aptamer and antibody plays a crucial role. A higher affinity antibody can strip the virus off the sensor surface, and lower concentrations of the antibody must be used to obtain a signal and detect the virus (see e.g., FIG. 10).

Elimination of wash step. The virus and antibody-GOx was premixed to a total volume of 200 μL (0.05 mg/mL of Ab-GOx+ virions in PBST). 200 μL of this solution was dropped onto the membrane discs and incubated for five minutes. The control sample did not contain any virus. The membranes were transferred to the electrodes, and 50 μL of glucose solution (e.g., 500 mM) was added, and chronoamperometry was performed. In the case of virus sample, the bound Ab-GOx was closer to the electrode surface than the free Ab-GOx in solution, as in case of the control sample. A higher current signal was observed in the case of viral sample showing that a close proximity of glucose-oxidase to the electrode surface produced a higher current signal.

Thus, in some embodiments, the wash step (e.g., removing the free Ab-GOx in solution) can be omitted. In some embodiments, in the lateral flow system the analyte flows over the detection region onto the wicking pad without a separate wash step.

### Example 2: Detection of rVSV-CoV-2 and SARS-CoV-2

Viral culture for rVSV-CoV-2: ATCC SARS CoV-2 S protein was transfected into HEK293 Ts for ˜2 hours using PEI (polyethylenimine). Cells were washed 2× with media to remove the VSV (vesicular stomatitis virus). When cells showed adequate cytopathic effect (˜48 hours post infection), the media was taken and spun down to remove cellular debris. The supernatant containing the virus was then aliquoted and stored at −80° C.

Viral culture for SARS-CoV-2: In culturing the virus, the first cell type choice was Vero cells, as these cells are susceptible to SARS-CoV-2 infection. Other cells such as HuH7 or other human cell type can also be used and are susceptible to infection. Infection of Vero E6 cells was carried out in phosphate-buffered saline (PBS) containing 50 μg/mL DEAE-dextran (diethylaminoethyl-dextran) and 2% fetal calf serum (FCS; BODINCO). The inoculum was added to the cells for 1 h at 37° C., after which cells were washed twice with PBS and maintained in Eagle's minimal essential medium (EMEM; LONZA) with 2% FCS, 2 mM L-glutamine (PAA LABORATORIES) and antibiotics (SIGMA). Viral titers were determined by plaque assay in Vero E6 cells.

Detection: For the sandwiched sensing assay, a rabbit polyclonal anti-SARS-CoV-2 spike glycoprotein antibody was used (ABCAM ab 272504). This antibody targets the spike (S) protein of SARS-CoV-2. Similarly, an aptamer targeting the S protein (e.g., as described by Song et al.), was used (IDT technologies). A nitrocellulose membrane with 0.45 μm pores was used (THERMOSCIENTIFIC). The membrane surface (e.g., 4 mm diameter discs) was functionalized with streptavidin-NC (from ENQUIRE BIOREAGENTS) by dropcasting 10 μL of the solution (1 mg/mL of streptavidin-NC in PBS) onto the membrane. The membrane was dried at 37° C. for 1 hour. The aptamer (100 μM) was folded at 95° C. for 5 min in 1 mM MgCl₂ in PBS and allowed to cool down to room temperature for 15 min. Next, the aptamer was diluted to working concentration in PBS, and 10 μL of 20 μM of biotinylated aptamer was drop-casted on the membrane for 1 hour. Following aptamer immobilization, the surface was blocked to prevent non-specific binding using 5 μL of 3% BSA, followed by washing with 200 μL of PBST (PBS+0.05% TWEEN 20). The virion stock was diluted to the desired concentration in DMEM (THERMOFISHER, #21063029; e.g., 1000, 100, or 10 viral particles per mL). 10 μL of the viral sample was added dropwise to the membrane with the aptamer and incubated for 15 min. The antibody conjugated GOx (Ab-GOx), synthesized using ABCAM'S LIGHTNING-LINK (GOx conjugate kit, #ab102887), was diluted in PBST, and 5 μL of the solution was drop-casted and spread on the membrane surface and incubated for 15 min. Next the membrane was washed using 200 μL PBST and transferred to DROPSENS 710 electrodes. The chronoamperometric measurements were performed after addition of 50 μL of 500 mM glucose solution. For the negative control sample, DMEM buffer (i.e., lacking virions) was used. Chronoamperometric measurements were performed at a potential of −0.2 V using a portable potentiostat, and current was monitored over time. A significantly larger current compared to the background current was observed.

Detection of VSV-CoV-2 pseudotype in buffer: For the detection of rVSV virus with the same spike proteins on its surface as SARS-CoV-2, a similar procedure to above was performed. For the sandwiched sensing assay, a rabbit polyclonal anti-SARS-CoV-2 spike glycoprotein antibody was used (ABCAM ab 272504). This antibody targets the spike (S) protein of SARS-CoV-2. Similarly, an aptamer targeting the S protein (e.g., as described by Song et al.) was used (IDT TECHNOLOGIES). A nitrocellulose membrane with 0.45 μm pores was used (THERMOSCIENTIFIC). The membrane surface (e.g., 4 mm diameter discs) was functionalized with streptavidin-NC (from ENQUIRE BIOREAGENTS) by dropcasting 10 μL of the solution (1 mg/mL of streptavidin-NC in PBS) onto the membrane. The membrane was dried at 37° C. for 1 hour. The aptamer (100 μM) was folded at 95° C. for 5 min in 1 mM MgCl₂ in PBS and allowed to cool down to room temperature for 15 min. Next, the aptamer was diluted to working concentration in PBS, and 10 μL of 20 μM of biotinylated aptamer was drop-casted on the membrane for 1 hour. Following aptamer immobilization, the surface was blocked to prevent non-specific binding using 5 μL of 3% BSA, followed by washing with 200 μL of PBST (PBS+0.05% TWEEN 20). The virion stock was diluted to the desired concentration in DMEM (THERMOFISHER, #21063029; e.g., 1000, 100, or 10 viral particles per mL). 10 μL of the viral sample was added dropwise to the membrane with the aptamer and incubated for 15 min. The antibody conjugated GOx (Ab-GOx), synthesized using ABCAM'S LIGHTNING-LINK (GOx conjugate kit, #ab102887), was diluted in PBST, and 5 μL of the solution was drop-casted and spread on the membrane surface and incubated for 15 min. Next the membrane was washed using 200 μL PBST and transferred to DROPSENS 710 electrodes. The chronoamperometric measurements were performed after addition of 50 μL of 500 mM glucose solution. No significant difference between control sample and virion sample was seen without a wash step. A wash step using a mild detergent (NP-40) was used, and no difference was observed between control sample and virion sample. When a wash with PBST (0.5% tween) was used, a significant difference in current signal was observed between the sample containing the virus and control sample. A significantly larger current compared to the background current was observed

Concentration of antibody: In the case of targeting the same protein by aptamer and antibody, the concentration of antibody is critical. The binding affinity of aptamer to spike protein is in the nM range whereas the antibody to spike protein is in the pM range. At a concentration of Ab-GOx (0.5 mg/mL), no significant difference between the control and viral sample was observed. More Ab-GOx was added in order to detect the virus (1 mg/mL), and no difference in the signal was seen. Upon adding a lower concentration of the Ab-GOx reagent (0.1 mg/mL), a signal was observed, and virus was detected. These results showcase the importance of the relative binding affinities of the aptamer and antibody to the virus. If the antibody has a higher binding affinity to the target protein relative to the aptamer, the viral particles are stripped away with the Ab-GOx from the aptamer during the wash step. Similarly, if the binding affinities are similar but an excess of the Ab-GOx is used, the viral particles are stripped away with the Ab-GOx from the aptamer during the wash step. Thus, the concentrations of capturing aptamer and Ab-GOx used require significant experimentation and optimization to find the optimal concentrations for performance. For detection of the spike protein of SARS-CoV-2, optimal performance is achieved when the Ab-GOx possesses a lower affinity for the target antigen than the aptamer or antibody linked to the test strip that captures the viral particle.

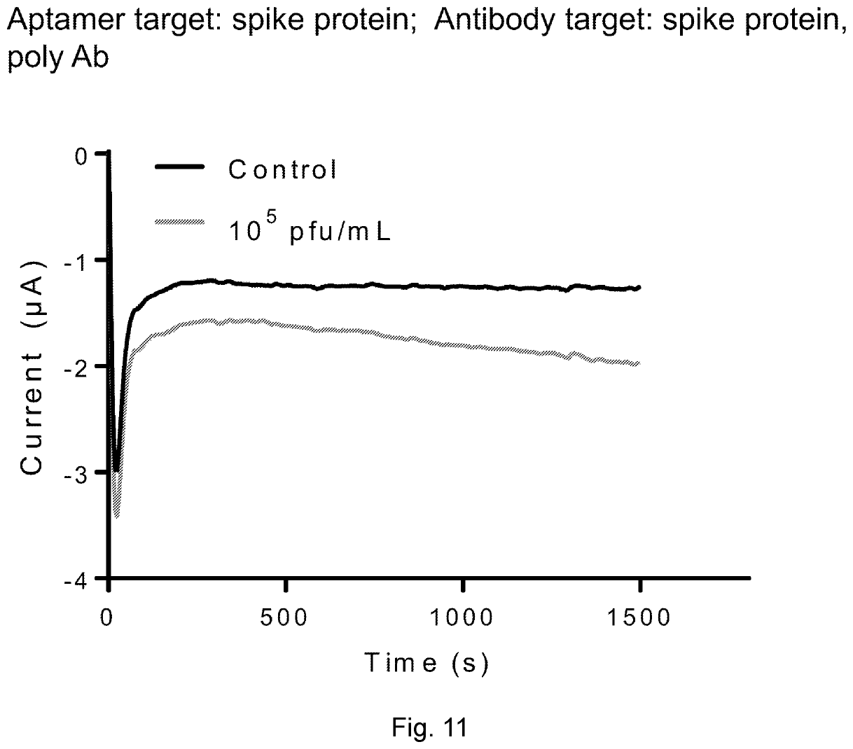

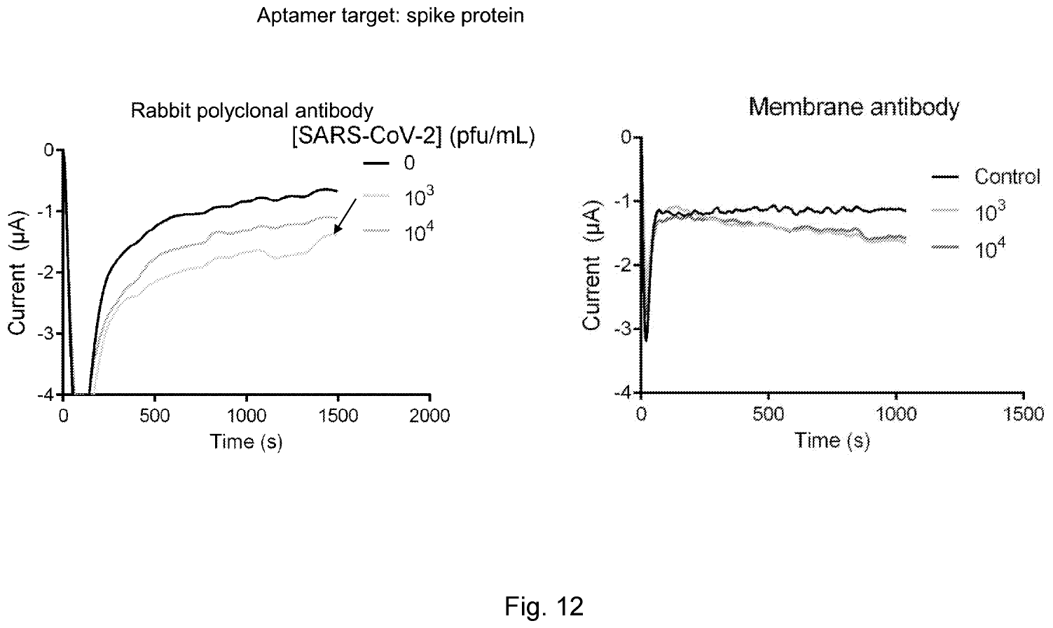

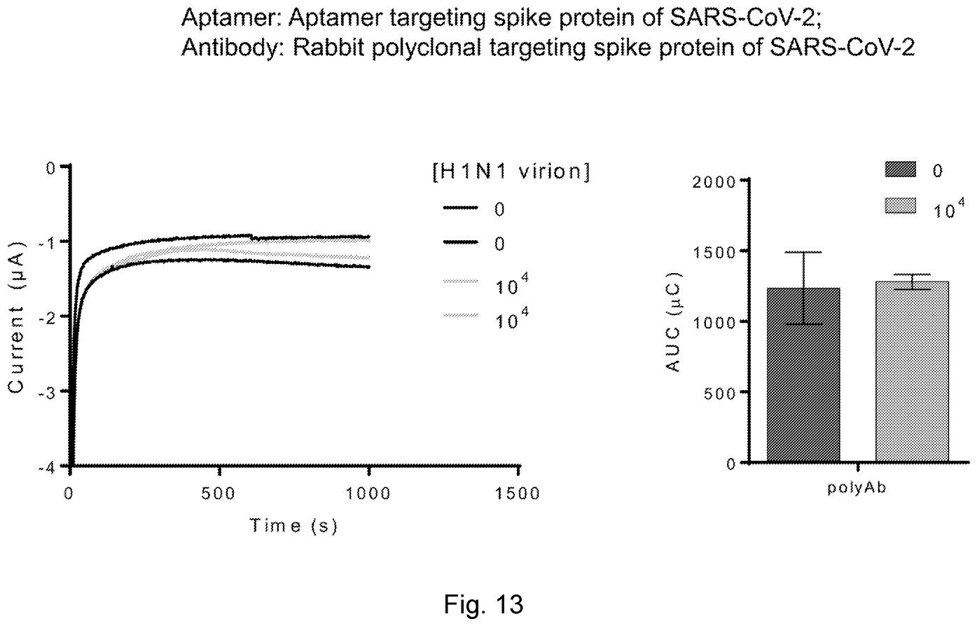

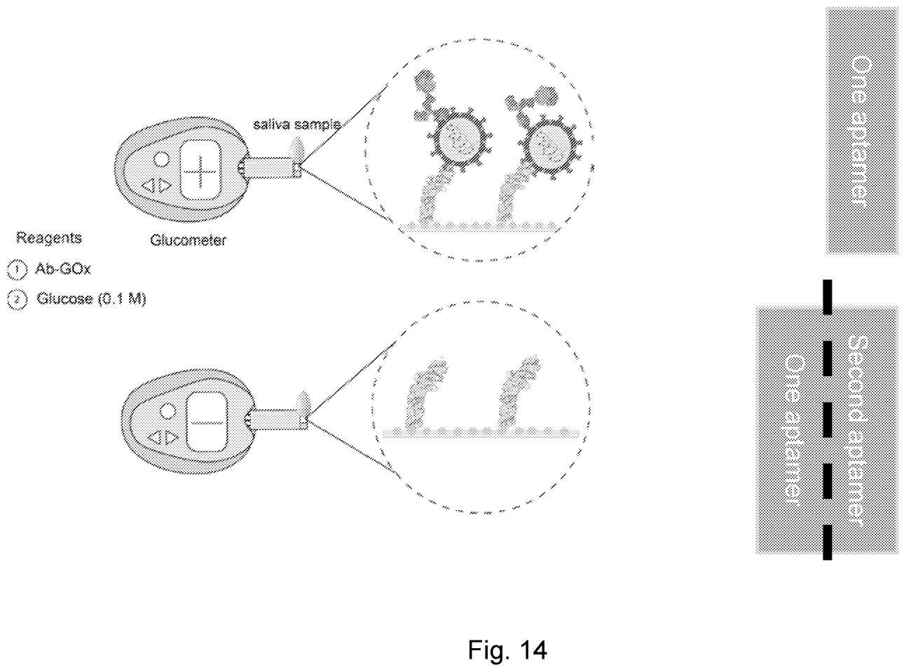

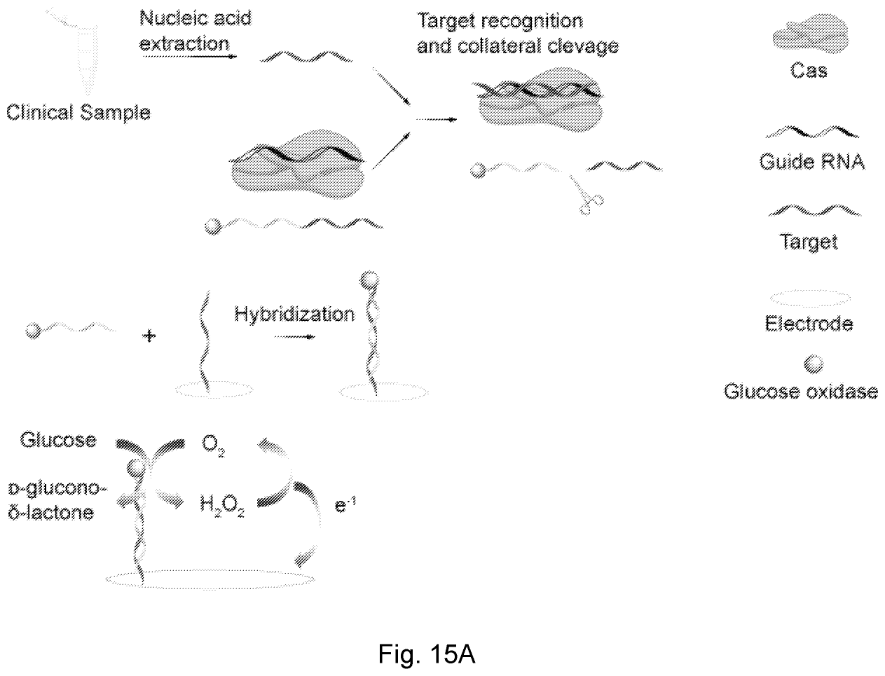

Comparison of different antibodies: Different antibodies and proteins targeting the spike protein and membrane protein were used. First, a rabbit polyclonal (ABCAM ab272504) and monoclonal antibody (ABCAM ab273433, 1A9) targeting SARS-CoV-2 spike protein were used. Second, since the spike protein of SARS-CoV-2 binds to ACE2 to mediate entry into host cells, an ACE2 (Angiotensin-converting enzyme-2)-Fc chimera was used (ABCAM #273687) and was tested against cultured SARS-CoV-2. Third, an antibody targeting the SARS-CoV-2 membrane protein was used (NOVUS BIOLOGICALS, Catalog #NB100-56569). All of the above antibodies and ACE2 were tested against SARS-CoV-2. All combinations provided a signal to detect compared to no virion control (see e.g., FIG. 11-13). The rabbit polyclonal antibody produced the maximum change in signal from the control baseline (see e.g., FIG. 11-13). The rabbit polyclonal binds to the C terminus of the SARS-CoV-2 spike protein and the aptamer targets the N-terminus reception binding domain of the spike protein. Having different epitopes for the aptamer and the antibody generates a stronger signal.

Aptamer immobilization strategies: Two aptamer immobilization methods were considered and investigated, one on nitrocellulose membrane and the second directly onto the screen printed electrode. The performance of each system can be evaluated by measuring the current generated by the oxidation of glucose using glucose oxidase under these conditions.

Immobilization method 1: In the case of direct immobilization on electrode, a thiolated aptamer can be used, and thiol-gold interactions can be utilized to immobilize the aptamer on a gold electrode. In order for the electrode to have both the mediator layer and gold for the aptamer immobilization, the mediator layer can be electrodeposited first and then the gold nanoparticles can be electrodeposited. A DROPSENS (DRP 510) electrode (Working Electrode (WE): Carbon, Counter Electrode (CE): Pt, Reference electrode (Ref): Ag/AgCl) can be used, and a Prussian blue layer can be deposited by dipping the electrode in 2.5 mM FeCl₃, 2.5 mM potassium ferricyanide, in 0.1 M HCl and applying a potential of 0.4 V for 40 s. This can be followed by conditioning of electrode in 0.1 M HCl+0.1 M KCl solution by applying a cyclic potential of −0.5 V to 0.35 V for 25 cycles at 50 mV/s. Gold nanoparticles can be electrodeposited by dipping into a solution of 100 mg/mL of HAuCl₄ and applying a potential of −0.2 V for 30 s. After the electrode preparation, the desired concentration of aptamer can be dropcast and incubated in a humidity chamber for 16 hours.

Immobilization method 2: For the immobilization on NC, the procedure as described in the first section will be followed using streptavidin-NC (streptavidin engineered to bind to nitrocellulose). The immobilization on nitrocellulose (NC) can provide a higher surface area and a more specific and stronger binding of the aptamer to the surface, but it can result in the addition of electrical resistance to the system.

Immobilization method 3: A solution of cellulose nitrate (SIGMA Catalog #09986-500ML) can be dropped on the working electrode containing the mediator and left to dry. The streptavidin-NC can be dropped, and the other reagents can be dropcast as mentioned in the immobilization method 2. The liquid cellulose solution can aid the transfer of electrons between the enzyme and mediator complex and can also result in an overall lower resistance of the system in comparison to the solid membrane. Under the same concentrations of aptamer-virion-Ab-GOx, the magnitude of current output can be compared to determine the optimal immobilization strategy.

Comparison of different mediators: There are different mediators used for the detection of glucose from human samples. Some examples of mediator complexes are iron-, osmium-, and ruthenium-based mediators. A thorough comparison of the different mediators can be performed by monitoring the current generated. The mediator that provides fast response and high current and requires low power requirements can be used to build the sensor strip.

Assessing the biosensor performance and validation: It is contemplated herein that sensors can be validated for expected performance in terms of current flux magnitude and velocities, appropriate dependence on redox enzyme, specificity, and dynamic range. Sensors can be optimized for critical performance metrics including analytical sensitivity and specificity, cross-reactivity, dynamic range, limit of detection, speed and duration of sensing, coefficient of variation of repeated measurements, and operational stability. Testing for sensor interference from compounds present in saliva using artificial saliva samples can be conducted. The performance of the device on commercially acquired human saliva samples can be tested and subsequently spiked with known concentrations of viral antigen, and then a calibration curve can be created. The samples can be used to determine the limit of detection (LOD) and sensitivity of the sensor using saliva samples. Statistical methods can be employed to design sample sizes for sufficient power, to determine confidence intervals, and to assess significance. Finally, testing can be performed to assess and calibrate sensor performance over a range of humidity levels, pH, and temperatures.

### Example 3: Repurposing a Commercial Glucometer to Detect SARS-CoV-2 Using a Test Strip

A SARS-CoV-2 test strip can be fabricated, e.g., using the optimization results from Examples 1-2, for use in a commercial glucometer to detect SARS-CoV-2. Laser cutter and screen-printing techniques can be used to fabricate the test strip. The electrode strip can undergo several rounds of optimization steps to meet design requirements using known concentrations of virus in artificial saliva (PICKERING SOLUTIONS, #1700-0313) before testing with human samples. Initially, the test strips can be tested using a lab potentiostat and compared to the commercially available electrodes, before incorporation into a glucometer. The glucometer can read glucose values that reflect the concentration of glucose oxidase enzyme present on the sensor surface. Different GOx concentrations can produce different glucose outputs on the glucometer for a fixed glucose concentration. The glucose values displayed on the glucometer (proportional to current output) can be calibrated to the concentration of viral particle present.

A point-of-care detection using a commercial glucometer can be used for sensing of viral infection. The sensor output depends on the concentration on antibody-GOx conjugate bound and hence in turn will depend on the viral antigen concentration. Knowledge of the relationship between glucose values and concentration of viral antigen can be used for the diagnosis of viral infection.

Fabrication of test strip: As shown in FIG. 5, the sensor strip comprises (bottom up): a (A) base substrate; (B) a conductive layer which includes three electrodes; (C) an insulating layer exposing only part of the electrode where the sample to be tested can be dropped; (D) a reagent layer containing mediator for ease of exchange of electrons; (E) an adhesive layer; (F) a hydrophilic nitrocellulose membrane, with a proximal membrane containing aptamer to capture the antigen and freeze dried glucose, and the distal end is the paper sink (13); (G) freeze-dried Ab-GOx; and (H) a top layer. See e.g., FIG. 5 and labels (A)-(H) and (1)-(16) therein. The base substrate can be polyester; an acrylic coating can be applied to improve the ink adhesion. Using a CAD (computer-aided design) model of electrode mask, the mask is laser cut onto the base substrate. The electrodes can be screen printed using conductive carbon inks (ERCON INC), followed by an insulation layer (ERCON INC, INSULAYER INK). The two working electrodes can have a surface area of 0.6 mm² each, and the reference electrode can have a surface area of 1.2 mm². The reagent layer is the mediator layer and can consist of a binder, silica, and ferricyanide. This reagent layer is screen printed for two cycles over the working electrodes. The adhesive layer on top can be an acrylic copolymer, and the hydrophilic membrane can be a nitrocellulose membrane with streptavidin-NC and biotinylated aptamer is bound (e.g., to streptavidin) to capture the viral antigen. The top layer can be PET (Polyethylene Terephthalate), with a small clear portion to see the sample movement on the strip. The overall dimensions can be the similar as described for other test strips, e.g., ensure compatibility with a glucometer, such as LIFESCAN reader, the test strip can be altered to be compatible with other commercial glucometers.

Biosensor evaluation in buffer and artificial saliva samples: Before the addition of freeze-dried bio reagents, and aptamer immobilization GOx, with or without antibody linked, can be directly deposited onto the working electrode, and the functioning of the disclosed test strip strips can be compared to commercially available glucose strips. This can be done by observing glucose readings displayed on the glucometer at constant glucose concentrations. Reagents can be immobilized to complete the fabrication of the SARS-CoV strip as mentioned in the previous section. The single use test-strips can be validated using viral samples in buffer and in artificial saliva. Around 100 μL of viral particles in solution can be dropped onto the sample chamber, and the solution navigates to the Ab-GOx layer. In presence of the viral particle, Ab-GOx binds forming a virion-Ab-GOx complex. This complex in turn migrates to the testing zone that contains glucose and aptamer. The aptamer present in the testing zone binds to the complex, bringing the GOx in close proximity to the working electrode and the glucose in solution. The test strip when inserted into the glucometer displays the corresponding glucose values. This process can be repeated for different concentrations of viral samples in buffer to build a calibration plot. The test can also be conducted in artificial saliva samples to test the effect of interferents in saliva. Blind samples with and without viral antigens can be tested to confirm the proper functioning of the test strips.

Storage and shelf life of the test strips: The tests above can be followed by more extensive testing and characterization in vitro to establish stability during activity, shelf-life, and tolerance to desiccation and lyophilization. Specifically, a freeze-drying method, with protective agents (e.g., mannitol) and disaccharides (e.g., sucrose, lactose, maltose, and trehalose), can be applied to elongate the shelf-life of biosensor. The protective agents can be added to the nitrocellulose membrane before addition of streptavidin-NC and aptamer. Biosensors can be subjected to a drying protocol different from conventional freeze-drying in that samples can be dried under reduced pressure in a dry atmosphere, but temperatures can be kept above freezing to avoid freeze-injury. Biosensors can be stored in fridge (4° C.), and room temperature (25° C.). Over a period of a month, these sensors' performance can be evaluated in terms of generation of current, compared to sensors without protective agents. Also accelerated aging on the strips can be performed to determine test stability over time. Standard accelerated aging protocols can be followed. Accelerated aging allows the estimation of a shelf life based on shorter incubation of a packaged device at higher temperatures. This method the industry standard, and can provide insight into any problems with choice of materials, surface chemistry changes, or assay stability. Initial tests can be performed at 45° C. for 80 days to estimate the 12-month shelf-life.

Exploring the scope of use as a POC device: Tests can be performed to optimize the time required for testing the viral samples. An ideal POC device provides results with 2 minutes. Glucose readings will can conducted using the glucose reader at different time intervals (t=1 min, 2 min, 5 min, 10 min) after the addition of saliva samples to investigate the effect of incubation time on the output. The effect of sample volume on the glucose output (100 μL, 250 μL, 500 μL, 1000 μL, 2000 μL) can also be determined.

### Example 4: Validating the SarS-CoV-2 Glucometer Using Human Saliva Samples and Comparing the Limit of Detection and Sensitivity to Those of RT-PCR

The device can be validated for analytical accuracy against current commercial assays like RT-PCR as well as lateral flow assays on commercially obtained human saliva samples spiked with viral antigen. The human samples spiked with viral copies ranging from 1 copy/mL to 100,000 copies/mL can be tested, and 95% confidence interval can be used to determine limit of detection. Cross-reactivity can be evaluated by testing various microorganism viruses and negative matrixes that might potentially interfere with the functioning of the device.

The point-of-care (POC) detection from saliva samples can give rapid and facile determination of viral infection in less 2 minutes compared to RT-PCR assays that take around 2 hours and require experienced lab personnel and expensive equipment. Nasal swabs used for RT-PCR assays are a cause of discomfort to the patients. Ease of obtaining the saliva samples is an advantage of this detection technology described herein. RT-PCR and lateral flow assays tests are reagent intensive and require expensive swabs whereas the testing described herein does not require multiple reagents or swabs as saliva samples will be collected in a sterile vial.

Evaluation using human saliva samples: The disclosed single use disposable point-of-care test strips in human saliva (INNOVATIVE RESEARCH INC.) can be evaluated by spiking in viral particles from concentrations ranging from 1 copy/mL to 100,000 copies/mL with n=5 for each concentration. A calibration curve can be built to find a relationship between glucometer reading and viral particle concentration. These results can be used to calculate the limit of detection of the device. In parallel, each sample can be validated using an RT-PCR assay to compare the LOD of the device against the gold standard assays available. Statistical methods can be employed to design sample sizes for sufficient power, to determine confidence intervals, and to assess significance. To achieve a 95% confidence interval for a maximum sensitivity and specificity of 95%, a sample set of 73 positive spiked samples and 73 negative spiked samples can be blinded and tested on 146 different strips.

Clinical evaluation: Commercially available human saliva samples with inactivated respiratory related pathogens like influenza strains, MERS-CoV, SARS-CoV, and Adenovirus can be evaluated to determine the cross-reactivity to other viruses; the device can also be tested against microorganisms like Mycoplasma pneumonia, Streptococcus pyogenes etc., that could potentially interfere with the device. 50 human saliva samples can be obtained from tested patients with 25 positive and 25 negative to SARS-CoV-2. The samples can be randomized, blind-labeled and tested using the disclosed system device and gold-standard RT-PCR assays to determine the percentage agreement between the two assays.

### Example 5: Determining Device Performance Against Mutant Coronavirus and Influenza Strains

Systematic evolution of ligands by exponential enrichment (SELEX, also referred to as in vitro selection or in vitro evolution) strategies can be used for identification of aptamers for the detection device, and established assays can also be used to identify antibodies for the detection device; see e.g., Darmostuk et al., Biotechnology Advances, (2015) 33, 6, 1141-1161, the content of which is incorporated herein by reference in its entirety. The sensor can also be repurposed to detect strains of influenza that affect millions of people a year.

### Example 6: Detection of IgG, IgM and IgA Produced by a Viral Infection

Detection: Upon infection, the host body mounts an immunological response to the infection with the production of IgG, IgM, and IgA. These immunoglobulins can be detected in a biological sample and provide information on the stage of viral infection of the individual. For the detection of these immunoglobulins, an anti-human IgG/IgM or a specifically screened aptamer can be immobilized on the sensor strip for the capture of these antibodies from a human sample. For the detection, a peptide sequence having affinity to an IgG or IgM antibody (e.g. viral antigen) can be conjugated with GOx. The peptide-GOx binds to the antibodies. The antibody then has a second binding event when it binds the aptamer or anti-human IgG/IgM linked to the test strip, bringing the GOx in close proximity to the working electrode surface. Production of hydrogen peroxide by the GOx can then be detected by the electrode.

It is contemplated herein that SARS-CoV-2 antibodies can be detected using a SARS-CoV-2 protein (e.g., spike protein) immobilized on the surface of the test strip and an anti-human IgG (or anti-human IgM, or anti-human IgA) antibody conjugated to glucose oxidase.

It is contemplated herein that H1N1 antibodies can be detected using an H1N1 protein (e.g., hemagglutinin protein immobilized) on the surface of the test strip and an anti-human IgG (or anti-human IgM, or anti-human IgA) antibody conjugated to glucose oxidase.

### Example 7: Detection of SarS-CoV-2 from Saliva Using a Commercial Hand-Held Glucometer

This disclosure is relevant to the emerging viral diseases focus area for the development of sensors that provide real-time diagnostics and can be used in a point-of-care (POC) setting for emerging viral diseases to predict illness before the onset of symptoms. To this end, described herein is a POC device that capitalizes on the commercial success and robustness of the glucometer to develop a rapid, facile, and quantitative diagnostic for SARS-CoV-2 in saliva.

BACKGROUND: Coronaviruses (CoVs) are enveloped viruses with spike glycoproteins on the surfaces that give a crown like appearance. In December 2019, a novel CoV outbreak which started in Wuhan, Hubei province, China, was identified and named the severe acute respiratory syndrome coronavirus 2 (SARS-CoV-2). The virus rapidly spread across the world causing the coronavirus disease-19 (COVID-19) pandemic and has, to date, infected more than 178 million people and killed more than 3.85 million individuals. Military personnel are at higher risk towards infection due to the high density and close contact of individuals at work (e.g., basic training, military bases, airlifts, ships and submarines, main and forward operating base for missions). This military working environment jeopardizes the ability to respond appropriately. In fact, major publications report that Military COVID-19 infection rate has surpassed the per capita U.S. rate.

Testing remains the key to identifying and isolating contagious individuals to minimize the spread of infection. It is imperative to screen and isolate both asymptomatic and pre-symptomatic individuals (e.g., soldiers). Reverse transcription polymerase chain reaction (RT-PCR), the gold standard for testing, has a high false negative rate and requires viral nucleic acid to detect the infection. This eliminates the use of serum and urine in RT-PCT tests, as these do not contain the viral nucleic acids, leaving only respiratory track samples such as nasopharyngeal swabs. In addition, the RT-PCR technique is not conducive to testing at military deployed bases or in the field, as it requires experienced technicians, extensive reagents and swabs and capital equipment and suffers from long turnaround times (days) to obtain results.

Several lateral-flow immunoassays have been developed for the detection of SARS-CoV antibodies. These tests look for the presence of three types of antibodies, namely IgG, IgM and IgA produced in the body as an immune response. However, these antibody-based assays are not useful for early diagnosis of infection as the body takes 5-10 days post-infection to produce these antibodies. Finally, antibody tests tend to suffer from sensitivity and specificity issues that result in a high false positive rate.

A transformative weapon in the fight against COVID-19 is a rapid, accurate point-of-care (POC) test for SARS-CoV-2. The US Defense Department has set a goal of testing 60,000 service personnel a day. A fail-safe POC device that is easy to commercialize, involves self-sample collection, and provides instantaneous test results, achieves this goal.

Described herein is a portable, rapid, and facile sensor for quantitative detection of SARS-CoV-2 from saliva which capitalizes on the commercial success and wide-spread use of the glucometer for measuring glucose. Specifically, described herein is a glucometer-based CoV test strip that comprises an aptamer for capturing the virus and, an antibody-glucose oxidase (Ab-GOx) conjugate for signal amplification in the presence of glucose. In this sandwich detection assay, the glucometer provides an electrochemical signal which correlates to the viral copy number present in saliva. Using saliva averts the need for nasal swabs, which can cause bleeding and have a higher risk of viral transmission. Saliva specimens can be readily collected by spitting into a vessel, the contents of which can then be applied to the test strip. Without wishing to be bound by theory, it is hypothesized that this system can sense SARSCoV-2 with a limit of detection of less than 10 copies/mL (see e.g., FIGS. 4, 6, 10 for exemplary results showing detection of influenza virus down to 10 PFU/mL; see e.g., FIGS. 11-13 for exemplary results showing detection of SARS-CoV-2). A glucometer-based SARS-Cov-2 sensor can also be adapted as quantitative POC devices for other viral infections.

Described herein is the design and assembly of the sensor, as well as the optimization and integration of the electrode assembly into a real-time assay. The immobilization procedure can be optimized with antibodies and aptamers specific to the virus to ensure maximal virus capture onto the surface. The sensors can be tested in a commercial laboratory-scale potentiostat using chronoamperometry in PBS and in artificial saliva samples spiked with SARS-CoV-2 virions and glucose (e.g., 0.1M). The specificity, selectivity, limit of detection of the assay and the effect of common interferents to the sensor output can be determined. The performance, assay time, and ease of use can be compared to the standard RTPCR assay.

Also described herein is the repurposing of a commercial glucometer to detect SARS-CoV-2 using a test strip. An electrode strip is built that mimics a glucose sensor strip for use in a standard, commercial, hand-held glucometer. The sensor surface consists of a nitrocellulose membrane that contains the capture aptamer for the viral antigen along with the freeze-dried reagents (Ab-GOx, glucose) required for signal generation, amplification, and subsequent detection. The performance of test strips is evaluated in a glucometer using PBS and artificial saliva to ensure the proper functioning of these strips and to assess the correlation between glucometer readings and the viral antigen concentration. The time required to run the diagnostic can be optimized. The storage and shelf-life of the sensor strips under different temperatures and humidity storage conditions can also be evaluated.

The SARS-CoV-2 glucometer can be validated using human saliva samples, and the limit of detection and sensitivity can be compared to those of RT-PCR. The device can be validated for analytical accuracy against RTPCR assays on commercially obtained human saliva and nasal samples. Human samples spiked with viral copies ranging from 1-10¹ copies/mL can be tested to establish the correlation between glucometer readings and virion concentration. Cross-reactivity against various microorganisms, viruses, and negative matrixes that may interfere with the functioning of the device can be evaluated. The device can be tested in a blinded manner using known positive and negative samples from donor patients.

Sensor performance against emerging coronavirus strains can also be tested. As it is highly likely that new or mutated CoVs will emerge within the next several years (e.g., at least 4 years), new glucometer-based test strips can be developed for these CoVs. Additionally, the test strips can be redesigned to sense other viral strains such as influenza, which still remains the greatest infectious threat to military activity each year.

The short-term impact of this work is the development, testing, and validation of a SARS-CoV-2 sensor which relies on the established principles of the common glucometer. Additionally, this technological development can be readily adapted to the detection of other CoV or respiratory viruses. In the long-term, an inexpensive, personal, facile, rapid-response device for detecting SARS-CoV-2 that can be used at home, at work, or in the field is of significant value for the world.

### Introduction: Detection of SARS-CoV-2 From Saliva Using a Commercial Hand-Held Glucometer

The novel coronavirus SARS-CoV-2 rapidly become a public health emergency as it spread to 213 countries and has taken more than 3.85 million lives since its origin in December 2019. The World Health Organization declared this coronavirus, a pandemic in March, 2020. Since then, the United States enacted self-quarantining, social distancing, and practicing good personal hygiene measures to contain the spread of the infection. The high infection rates completely overwhelmed the hospitals and clinics as they faced severe shortages in beds, ventilators, and many doctors and medical health professionals succumbed to the virus. Not only has the coronavirus impacted the general public, but also brought many important military installations and missions to a standstill. The high density and living conditions in close quarters along with the close personal contact required during deployment put military personnel at even higher risks of infection than the general public.

The Military, along with national health experts, have continuously stressed the need for expanded testing capabilities. Further, the testing device must be easy to use, provide an answer within sixty seconds, require a simple-to-obtain bodily fluid—such as saliva—require no complex training, and be readily deployable in any environment. The solution described herein to this problem builds upon the common glucometer—an over-the-counter device sold at major pharmacy stores and used by millions of diabetic US citizens every day. Diabetics must measure their blood glucose level multiple times a day and, each time requires the use of a glucometer. The individual applies a small drop of blood to a test strip and then inserts it into the glucometer. The glucometer performs an assay and provides a nearly instantaneous result. This diagnostic platform can be re-purposed herein for the detection of SARS-CoV-2.

Specifically, described herein is a test strip that uses saliva, rather than blood, but is still compatible with standard over-the-counter commercial glucometers. The glucometer provides a Yes/No result as to whether the sample, and hence the person, is SARS-CoV-2 positive or negative. This idea permits the use of a standard glucometer and requires only the development of a virus-specific test strip. This approach avoids the use of nasal swabs for sample collection which, for anyone who has not experienced it, is a most unpleasant experience. Importantly, data support this approach, and well-characterized materials and rigorous experimental designs are established herein.

At present, there are few approved POC devices that allow self-testing at home, in the office, or in operational field with the low limit of detection and high accuracy required to be truly useful. Accuracy is of the upmost importance as a false negative result could lead an infectious individual to believe they do not have SARS-CoV-2 and, subsequently, to unknowingly infect individuals around them. POC testing is essential for rapid detection of the infection at early stages to facilitate better disease diagnosis, monitoring and management. The innovative material, approach, and device concepts are: 1) detection of viral antigen in human saliva; 2) straightforward saliva sample collection; 3) use of an existing glucometer technology that is widely available and inexpensive; 4) the test strip is easily adaptable to the detection of other viruses; and, 5) excellent commercial viability as the test strip is the only new component of a technology (the glucometer) and practice (viral testing) already widely used in society.

The overall approach represents a paradigm shift in the design of biosensors with far reaching benefits for the development of new biosensors.

The detection device described herein can yield highly impactful data leading to critical discoveries and major advancements in detection technology for respiratory viruses including SARSCoV-2. The device provides the opportunity to increase basic knowledge on collection, detection, and signal generation for POC sensors and diagnostics. Militarily, this transformative approach offers a significant advancement to testing and monitoring for service men and women who are at elevated risk for infection due to their job responsibilities and commitment to protecting our nation.

The detection device described herein can help accomplish many scientific. The novel coronavirus designated as respiratory syndrome coronavirus-2 (SARS-CoV-2), originated in Wuhan, China. Researchers around the world are racing to develop vaccines and treatment therapies to save lives and minimize the impact of this pandemic. Meanwhile, testing and diagnosis of SARS-Cov-2 is the key to identification and isolation of infected individuals; comprehensive testing is the best approach to identify patients for quarantine and/or early interventions for those with underlying conditions likely to increase the severity or mortality of their case. Described herein is a portable, rapid, and facile sensor that permits quantitative detection of SARS-CoV-2 from saliva and which capitalizes on the commercial success and wide-spread use of the glucometer for measuring glucose. The detection device (also referred to herein as a sensor) eliminates the use of uncomfortable sample collection techniques, such as nasal swabs, and provides a Yes/No diagnosis at the point-of-care. Specifically, the CoV test strip replaces the generic glucose strip used for measuring glucose in a commercial glucometer, to test SARS-CoV-2. The glucose reading displayed on the glucometer corresponds to a particular viral particle concentration and, thus, this provides quantitative detection of the viral infection. This repurposed glucometer can sense SARS-CoV-2 with a limit of detection of less than 10 copies/mL. This CoV test strip is the first quantitative POC device for the detection of SARS-CoV-2.

The gold standard for SARS-CoV-2 testing is based on reverse transcriptase-polymerase chain reaction (RTPCR) and relies on using respiratory samples, experienced personnel, extensive stocks of reagents, expensive equipment and is riddled with high false negatives. Currently, there are around 80 FDA approved RT-PCR kits with an average limit of detection of around 100 RNA copies/mL. In order to transform the rate and quantity of testing, it is imperative to move diagnostics from laboratory settings to the point-of-care (POC). Accurate and scalable POC devices that provide instantaneous test results increase the scope of tracking the spread of the disease in communities and thereby support early isolation and control measures. The current POC devices include lateral flow assays for antibody detection that have high false positive rates, low specificity, and do not offer quantitative detection. These reagent intensive gold standard assays for SARS-CoV-2 detection can be replaced with a simple, easy-to-use, off-the-shelf glucometer, as described herein.

Infectious diseases are caused by microorganisms such as viruses, bacteria, and fungi. These diseases have the capability to spread exponentially among populations with a high mortality rate and to cripple the world economy while pushing to the limit governments' ability to respond correctly and appropriately. The current coronavirus pandemic that has spread to 213 countries and has taken more than 3.85 million lives is a devastating example of this danger-one not seen in the Western world in nearly a hundred years since the outbreak of the Spanish Flu during WWI. Irrespective of the SARS-CoV-2 pandemic, the rate of infectious disease spread was already on the rise throughout the world. Described herein is a detection device that capitalizes on the success of the glucometer, which is a readily translatable detection technology. Currently, glucose meters dominate 60% of the world market for POC diagnostics. By repurposing this commercialized and facile detection method, there can be a significant impact on the current testing methodologies for viral diseases. Further, it is highly likely that new or mutated CoVs will emerge within the next several years (e.g., 4 years) and, thus, new glucometer-based test strips can be developed for these viruses. For example, the CoV test strip will easily be repurposed to sense other viral infections like seasonal flu which affects 8% of the US population every year.

This disclosure is relevant to the emerging viral diseases focus area for the development of sensors that provide real-time diagnostics that can be used as a point of care (POC) for emerging viral diseases to predict illness before onset of symptoms. To this end, described herein is a POC device that capitalizes on the commercial success of the glucometer for quantitative detection of SARS-CoV-2.

Coronavirus Transmission. Coronaviruses (CoVs) are enveloped viruses with spike glycoproteins on the surfaces that give a crown like appearance. In December 2019, a novel CoV outbreak, which started in Wuhan, Hubei province, China, was identified and named the severe acute respiratory syndrome coronavirus 2 (SARS-CoV-2). The virus rapidly spread across the world causing the coronavirus disease-19 (COVID-19) pandemic and has, to date, infected more than 178 million people and killed more than 3.85 million individuals. The SARS-CoV-2 pandemic demonstrates the vulnerability of the United States' military and healthcare system to new viruses. While there are many state-of-the-art detection techniques for sensing SARS-CoV-2, there are few point-of-care devices (POC) that involve easy sample collection techniques, and self-testing at home, in the office, or during active duty. This prevents the expansion of widespread testing that is critical in stopping the looming pandemic.

Current Detection Strategies. Testing remains the key to identify and isolate the individuals expressing the contagion to minimize the spread of infection. In many countries that successfully contained the infection, diagnostics have remained the cornerstone for success. Reverse transcription polymerase chain reaction (RT-PCR), the reference standard for testing, has a high false positive rate and requires viral nucleic acid to detect the infection. This eliminates the use of serum and urine that is negative for the nucleic acid and resorts to respiratory track samples using nasopharyngeal swabs, for example. Not only are the swabs invasive but their use also puts the healthcare workers at higher risks of infection. The RT-PCR technique is also not conducive to large-scale testing and commercialization since it requires experienced technicians, extensive reagents and swabs, expensive equipment, and long turnaround time for results. Several lateral-flow immunoassays have been developed for detection of SARS-CoV antibodies. These tests look for the presence of three types of antibodies, namely IgG, IgM, and IgA, produced in the body as an immune response. But, these antibody-based assays are not useful for early diagnosis of infection as the body takes 5-10 days post-infection to produce these antibodies. The antibody tests tend to have sensitivity and specificity issues that also result in a high false positive rate. One of the key weapons for the fight against COVID-19 is effective testing by commercialization of POC devices to test SARS-CoV-2. Of paramount importance for world governments and their departments (e.g., the Military) is to have ready access to these tests to identify early stages of infection and adopt proper quarantine measures. An ideal POC device for SARS-CoV-2 is a fool-proof device to be used at home, in the office, in the field, or when deployed on a mission that involves self-sample collection with instantaneous test results.

TABLE 1
Table showing FDA approved kits for detection of SARS-CoV-2; NS =
nasal swab, OPS = oropharyngeal swab, NPS = nasopharyngeal
swab, TS = throat swab, TA = tracheal aspirate, BAL =
bronchoalveolar lavage, NPA = nasopharyngeal aspirate.

| | | | | False |
|---|---|---|---|---|
| Detection | | | LOD | positive/false |
| Method | Kits | Sample | (copies/mL) | negative |
| RT-PCR/ | ABBOTT REALTIME | NS, OPS, NPS | 100 | 33.3% |
| Molecular | ID NOW - ABBOTT | NS, TS, NPS | 125 | |
| | PERKIN ELMER | NS, OPS | 20 | |
| | AURORA BIOMED | NPS, sputum | 200 | |
| | QIASTAT-DX - | NPS | 500 | |
| | QIAGEN | | | |
| | NEWMODX | NS, NPS, OPS | 150 | |
| | QUEST DIAGNOSTICS | NPS, OPS, TA, BAL | 137 | |
| | BIOFIRE | NS | 330 | |
| | GENESIG | OPS | 330 | |
| | PIXEL-LAB CORP. | NS | 6250 | |
| | CDC Diagnostic Panel | NPS, OPS, BAL, sputum, | 1000 | |
| | | NPA | | |
| Antigen | SOFIA SARS | NS, NP | 1.13*10² | 20% |
| | ANTIGEN FIA - | | TCID₅₀/mL | |
| | QUIDEL | | | |
| | CORP. | | | |

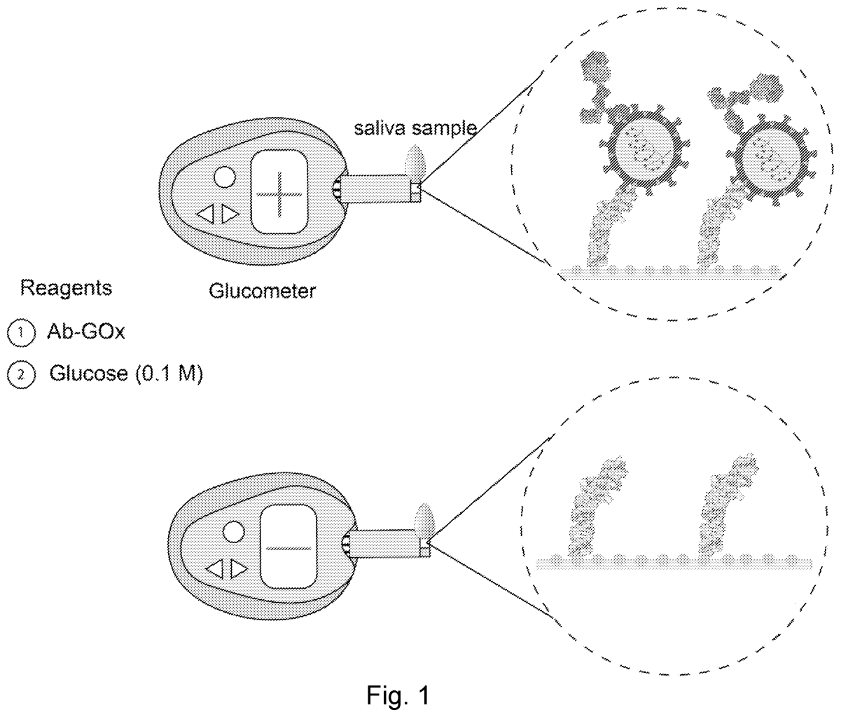

Described herein is a portable, rapid, and facile sensor for quantitative detection of SARS-CoV-2 from saliva, that leverages the commercial success and wide-spread use of the glucometer for measuring glucose. The viral antigen can be detected in saliva, thus avoiding use of nasal swabs that cause nasal bleeding and increase the risk of transmission. Saliva specimens can be easily collected by spitting on a strip or in a vial. Specifically, described herein is a glucometer-based CoV test strip using screen printing techniques that can be subsequently read or analyzed using a commercial glucose reader (see e.g., FIG. 1). The CoV strip comprises an aptamer for capturing the virus and an antibody-glucose oxidase (Ab-GOx) conjugate for signal amplification in the presence of glucose. In this sandwiched detection assay, the glucometer provides an electrochemical signal which correlates to the viral copy number present in saliva. One highlight of the detection device is the low cost and ease of commercialization of this device without the need of extensive reagents for sensing. Further, this technology is easily adaptable to detect new or emerging viral strains for the future or any strain of respiratory virus.

At present, there are no FDA approved POC devices that allow self-testing at home with low limit of detection and high specificity. POC testing is essential for rapid detection of the infection at early stages to facilitate better disease diagnosis, monitoring and management. POC tests can supplement laboratory-based testing, permitting testing to be available to large communities and populations that do not have access to laboratory testing.

Described herein is the repurposing of the widely used glucometer as a POC device to sense SARS-CoV-2. The sensing technique detects the viral antigen in human saliva, and thus avoids the use of nasal swabs for sample collection. FIGS. 1-2 summarize the proposed mechanism for sensing the viral antigen using saliva samples from patients.

Benefits of the device described herein include, but are not limited to: (1) detection of viral antigen in human saliva; (2) straightforward sample collection; (3) unique design to capture the viral antigen; (4) ease of commercialization; (5) quantitative detection of SARS-Cov-2; (6) high stability and specificity of aptamers; (7) GOx responds rapidly to glucose permitting rapid sensing; (8) high sensitivity and specificity to viral antigen target; (8) capable of being repurposed to sense other viral antigens by using a virus-specific aptamer and antibody; (9) reagents are freeze-dried to extend the shelf-life of the SARS-Cov-2 test strip; (10) readily integrated into low cost microfluidic and microelectromechanical (MEMS) devices; and (11) the overall approach represents a paradigm shift in the design of biosensors with far reaching benefits for the development of new biosensors

### Results

Viral culture for influenza. The cell culture and passage was performed according to the WHO's MDCK cell culture protocol; see e.g., Who, Manual for the laboratory diagnosis and virological surveillance of influenza. World Health Organization 2011 (2011), the content of which is incorporated herein by reference in its entirety. After the cells reached 70%-80% confluent, the DMEM media was aspirated and cells were washed three times in 5 mL of 1×PBS. The virion stock of influenza H1N1 A/Puerto Rico/8/34 (A/PR/8/34) was thawed in a 37° C. bath, and 500 μL-1000 μL of virion specimen was inoculated onto a T-75 flask. The flask was slowly tilted and rotated to spread the viral inoculum. The inoculum was allowed to adsorb for 30 min in the 37° C. incubator. 12 mL of viral growth DMEM (containing TPCK-Trypsin) was added into the same T75 flask. This was incubated at 37° C. and inspected daily for cytopathic effects (darkening of flask due to lysis). When the cytopathic effects were around 75%-100%, 12 mL of supernatant was collected using a serological pipette. 15% glycerol was added to the final solution, and aliquots were frozen at −80° C. for storage. The stocks had a concentration of 5×10⁶ PFU/mL measured via plaque assay and 1.64×10⁸ RNA copies/mL via RT-PCR assay.

Design of the sensor. Described herein is a sandwiched electrochemical detection mechanism consisting of an aptamer and an antibody labeled with glucose oxidase (GOx), both of which bind the virus of interest. As shown in FIG. 2, the aptamer captures the viral antigen, and Ab-GOx will bind only if viral antigen is present. GOx is a widely studied enzyme used in glucose biosensors due to its high enzymatic activity and stability.

On application of a constant potential, GOx oxidizes glucose, transfers an electron to oxygen, produces hydrogen peroxide, and generates a current output via an electrode that reacts with hydrogen peroxide. The electrode of sensor includes a mediator layer (e.g., Prussian blue) to lower the overvoltage of hydrogen peroxide production process and generate an output current at a lower potential.

Detection of H1N1 Influenza. For the sandwiched sensing assay, an anti-influenza A H1N1 neuraminidase antibody was used (ABCAM). This antibody has high affinity towards the neuraminidase proteins on the viral membrane. Similarly, an aptamer having affinity towards the hemagglutinin protein was used (IDT TECHNOLOGIES); see e.g., Kiilerich-Pedersen et al. Biosensors & bioelectronics 49, 374-379 (2013), the content of which is incorporated herein by reference in its entirety. A nitrocellulose membrane with 0.45 μm pores was used (THERMOSCIENTIFIC). A 4 mm in diameter hole was punched in the membranes. The membrane was functionalized with streptavidin-NC by dropcasting streptavidin (1 mg/mL in PBS) onto the membrane. The membrane was dried at 37° C. and stored under dry conditions overnight before use. Next, 10 μL of 20 μM of biotinylated aptamer was dropcast for 30 min followed by washing in PBS buffer. Following aptamer immobilization, the surface was blocked by adding 0.1% BSA on the surface, followed by washing. The virion stock from −80° C. was thawed, and centrifuged at 3000 rpm to remove cellular debris. Two ten-fold dilutions were performed on the supernatant virion stock to obtain 10⁵ pfu/mL, and 10⁴ pfu/mL of virion concentration. 5 uL of these solutions were added dropwise to two different membranes with aptamer and incubated for 15 min. The GOx conjugated antibody, synthesized using ABCAM'S LIGHTNING-LINK (GOx conjugate kit, #ab102887), was dissolved in PBST and dropcast and spread on the membrane surface and incubated for 15 min. Next the membrane was washed using 200 μL PBST and transferred to DROPSENS 710 electrodes. The chronoamperometric measurements were performed after addition of 50 uL of 500 mM glucose solution. For the negative control sample, instead of adding the virion stock, DMEM solution (0.2% BSA, 25 mM HEPES, 2 μg/mL TPCK-Trypsin) was used. Chronoamperometric measurements were performed at a potential of −0.2 V using a DROPSENS potentiostat (DRP-STAT-4000) and current was monitored over time. FIG. 4 (left) shows that as the virions were present in the sample, the bound glucose oxidase oxidized glucose and produced a current signal, whereas in the control sample case (no virions were present), negligible current was measured. Area under the curve of a current vs. time plot gives the total charge transferred, and FIG. 4 (top right and bottom right) shows that as the virions were present, charge transferred was an order of magnitude higher than in the case of control sample. Thus, a significant current signal was observed in presence of physiologically relevant viral concentration (e.g., 10⁴ pfu/mL), which can be replicated for SARS-CoV-2 using the corresponding antibody-aptamer combination.

The following experiments test the following hypotheses that: 1) an electrochemical sandwiched based assay for the detection of viral particles can be fabricated on a test strip for readout using a commercial glucometer; 2) the output from the glucometer can quantitatively correlate to the number of SARS-CoV-2 particles in saliva; 3) this repurposed glucometer can sense SARS-CoV-2 with a limit of detection of less than 10 copies/mL with a false negative rate of <1%. Further, test strips can be prepared for current and future coronavirus strains as well as other respiratory viruses (e.g., influenza).

To this end, these studies include development of a sandwiched electrochemical sensing system that comprises an aptamer and an antibody-glucose oxidase (Ab-GOx) conjugate and a repurposed commercial glucometer for SARS-Cov-2 detection.

Design and assembly of the sensor; optimization and integration of the electrode assembly into a real-time assay. The immobilization procedure with the antibodies and aptamers specific to the virus can be optimized to ensure maximal viral antigen capture onto the surface. The sensors can be tested in a commercial laboratory-scale potentiostat using chronoamperometry in PBS buffer and in artificial saliva samples spiked with SARS-CoV-2 virions. The specificity, selectivity, limit of detection of the assay and the effect of common interferents to the sensor output can be evaluated. The performance, assay time, and ease of use of the device described herein can be compared to the standard RT-PCR assay.

Repurposing a commercial glucometer to detect SARS-CoV-2 using a test strip. Described herein is an electrode strip that mimics a glucose sensor strip for use in a commercial standard hand-held glucometer. In one embodiment, the sensor surface consists of a nitrocellulose membrane with the capture aptamer for the viral antigen along with the freeze-dried reagents (Ab-GOx, glucose) required for signal generation, amplification, and subsequent detection. The performance of the test strips in a glucometer can be determine using PBS buffer and artificial saliva to ensure the proper functioning of these strips and to assess the correlation between glucometer readings and the viral antigen concentration. The time required for the diagnostic test can be optimized, and the storage and shelf-life of the sensor strips can also be evaluated under different temperatures, and humidity levels.

Validating the SARS-CoV-2 glucometer using human saliva samples and compare the limit of detection and sensitivity to those of RT-PCR. The device can be validated for analytical accuracy against RT-PCR assays on commercially obtained human saliva and nasal samples. Human samples spiked with viral copies ranging from 1-10¹ copies/mL can be tested to establish the correlation between glucometer readings and virion concentration. Cross-reactivity against various microorganisms, viruses, and negative matrixes that might potentially interfere with the functioning of the device can be evaluated. The device can also be tested with blindly labeled positive and negative samples from patient donors.

Determining sensor performance against emerging coronavirus strains. As it is highly likely that new or mutated CoVs will emerge within the next several years (e.g., 4 years), new glucometer-based test strips can be developed for these CoVs.

DESIGN REQUIREMENTS: The following set of performance criteria are essential to a successful coating based on the following design requirements: 1) sensitive to the target viral antigen in the concentration ranges for human saliva with a dynamic range over >5 logs; 2) LOD of the same order as RT-PCR assays of 10 copies/mL or lower; 3) exhibits a calibration curve of glucometer reading vs. concentration of viral antigen with an R²≥0.99; 4) displays less than 5% cross-reactivity to other viral strains and pathogens; 5) displays <10% coefficient of variation over the concentration range of viral antigen; 6) analysis time of <1 min; 7) stability and shelf-life of electrode strips and embedded reagents for more than 1 year; 8) negligible effect of common interferents in the saliva samples; and 9)<1% false negative and false positive rates.

A working laboratory assay for detection of SARS-CoV-2 can be built, that involves binding studies of aptamers and antibodies to the viral antigen; immobilization studies and validation of biosensor performance. The LOD of the assay can be 10 copies/mL; there is little to no interference in artificial saliva; a calibration curve can be performed of current output vs. virion concentration; there can be <10% coefficient of variation over the concentration range; and in one embodiment the duration of test is <15 min.

A SARS-CoV-2 test strip is designed and fabricated to be used in a glucometer; the relationship between the glucometer reading and the viral antigen concentration can be assessed. The CoV strip can work in a commercial glucometer; a calibration curve can be performed of glucometer vs. viral antigen concentration in buffer and artificial saliva (dynamic range >4-10 logs); the shelf life of test strips can be >1 year; in one embodiment, the time for testing can be <5 min.

The SARS-CoV-2 test strip can be validated using patient samples, which can be used to draw a comparison to gold standard assays. It is contemplated herein that the device can be sensitive to the viral antigen in the concentration ranges for human saliva with a dynamic range over >4 logs; the LOD can be 10 copies/mL; it is contemplated herein that a calibration curve of glucometer reading vs. virion concentration can be produced exhibiting a R²=0.99; the device can display less than 5% cross-reactivity to saliva interferents, other viral strains, and pathogens; the device can display <10% Coefficient of variation over the range of virion concentration; there can be a <1% rate of false positive and false negatives.

Sensor performance towards other viral strains can be evaluated by modifying the sensor parts (e.g., aptamer, antibody). Without wishing to be bound by theory, there can be similar detection performance against HIN1 and other CoV as well as other respiratory viruses.

A. Described Herein is the Design and Assembly of the Sensor, and Optimization and Integration of the Electrode Assembly into a Real-Time Assay.

A sandwiched electrochemical sensing strategy can be applied for the detection of SARS-CoV-2. A chronoamperometric measurement can be used to monitor the oxidation of glucose and the output current will be recorded. Aptamers and antibodies can be used that exhibit strong binding affinities to the proteins present on the viral membrane of SARS CoV-2; see e.g., Song et al., Analytical Chemistry, 2 Jul. 2020, 92(14):9895-9900; Yuan et al. Science 633, eabb7269 (2020); Wang et al. Nature Communications 11, 1-6 (2020); the contents of each of which are incorporated herein by reference in their entireties. Binding studies of aptamer-protein and antibody-protein interactions can be conducted using bio-layer interferometry to confirm the binding affinity. The identification of aptamer-antibody combination can be followed by assembling into a biosensor for the detection of viral antigen. Various strategies for immobilization of aptamer to the biosensor surface (e.g., direct immobilization on electrode, indirect immobilization on nitrocellulose (NC) membrane) can be incorporated. For the synthesis of antibody-glucose oxidase conjugate (Ab-GOx), different synthesis schemes can be extensively studied. Commercially available conjugation kits or direct fusion of Ab-GOx can be used, followed by expression to have minimal effect on the antibody-epitope binding affinity. The antibody-aptamer pairing can be validated by testing with other viral strains from the coronavirus family. After choosing the optimal combination, the specificity, selectivity, limit of detection, and other parameters can be optimized. The effect of common interferents in human saliva samples on the sensor output can also be studied. Described herein is a laboratory based assay to detect SARS-CoV-2 which can be modified into a point-of-care.

The binding affinities of aptamer and Ab-GOx to the viral proteins determine the sensitivity of the biosensor. The concentration of GOx bound to the sensor surface provides a current signal that is proportional to the number of viral particles as there is an excess of glucose present for the GOx. The high enzyme activity of GOx permits the detection of low concentrations of virus as the enzyme amplifies the signal.

The detection device described herein demonstrates that: 1) viral particles can be captured to the test strip surface using an aptamer which possess a high binding affinity to a viral envelope protein; 2) only the presence of viral particles generates a signal be as result of the binding of Ab-GOx forming a viral sandwich between the antibody and the aptamer; and, 3) on application of a constant potential, the GOx generates a current signal directly proportional to the number of viral particles in presence of excess glucose.

Viral culture: In culturing the virus, the cell type choice initially is Vero cells, as they are susceptible to SARS-CoV-2 infection, HuH7 or other human cell type can be used if susceptible. Infection of Vero E6 cells is carried out in phosphate-buffered saline (PBS) containing 50 μg/ml DEAE-dextran and 2% fetal calf serum (FCS; BODINCO). The inoculum is added to the cells for 1 h at 37° C., after which cells can be washed twice with PBS and maintained in Eagle's minimal essential medium (EMEM; LONZA) with 2% FCS, 2 mM L-glutamine (PAA) and antibiotics (SIGMA). Viral titers can be determined by plaque assay in Vero E6 cells as described previously; see e.g., van den Worm, et al. PLOS ONE 7, e32857 (2012).

1. Binding studies using Bio-Layer Interferometry (BL). Bio-layer interferometry can be used to study the binding affinities of aptamer-spike protein and antibody-spike protein and to calculate the corresponding dissociation constants. For the aptamer-spike protein study, biotinylated oligos containing the protein binding site can be used (INTEGRATED DNA TECHNOLOGIES, INC.). Streptavidin-coated biosensor tips are dipped into wells containing the binding buffer. The tips can then be moved into wells containing the oligo and allowed to bind the probe for 55 seconds. The probe can be moved back into the buffer for another baseline reading. The DNA-coated probe can be moved to the well containing the spike protein to test the binding of spike protein.

This test has multiple probes dipped into different wells containing different concentrations of the proteins. The probes can later be moved to a well containing buffer after the system reaches saturation. The dissociation constants can be calculated using the BLI software options.

Similarly, for antibody-protein interaction study, Anti-penta-HIS tips are used for capturing the antibody onto the BLI probe. Similar steps as described above can be followed to calculate the binding affinity of antibody-protein interaction. This test can be repeated with the antibody-GOx conjugate to investigate if the conjugation affects binding affinity to its epitope. Ideal choice of aptamer and antibody have <50 nM dissociation constants to the corresponding proteins. This study evaluates the different choices of aptamers and antibodies, in order to choose the optimal antibody and aptamer to build the biosensor.

2. Validation of aptamer-antibody pairing. It is contemplated herein that the specificity of aptamer and antibody interactions towards the proteins present on the viral envelope can be evaluated. This test can be performed using an aptamer having no binding affinity towards the viral protein. This non-binding can be confirmed prior with BLI. The non-specific aptamer can be immobilized followed by the addition of viral antigen and the specific Ab-GOx. There can be minimal current output as the viral antigen does not bind to the sensor surface due to the absence of specific binding sites. Similarly, an antibody with no affinity to SARS-CoV-2 spike protein can be used. After synthesizing and studying the Ab-GOx using BLI to confirm no preferential binding, the sample can be used in the sensing system to measure the current output. Again, there can be negligible current output as the Ab-GOx does not bind to the sensor surface owing to no binding affinity to the viral proteins.

3. Immobilization strategies. At least two aptamer immobilization methods can be considered, one on nitrocellulose membrane and the second directly onto the screen printed electrode. The performance of each system can be evaluated by measuring the current generated by the oxidation of glucose using glucose oxidase under these conditions.

Immobilization method 1: In the case of direct immobilization on electrode, a thiolated aptamer can be used, and thiol-gold interactions can be utilized to immobilize the aptamer on a gold electrode. In order for the electrode to have both the mediator layer and gold for the aptamer immobilization, the mediator layer can first be electrodeposited and then the gold nanoparticles can be electrodeposited. A DROPSENS (DRP 510) electrode (WE: Carbon, CE: Pt, Ref: Ag/AgCl) can be used, and Prussian blue layer can be deposited by dipping the electrode in 2.5 mM FeCl₃, 2.5 mM potassium ferricyanide, in 0.1 M HCl and applying a potential of 0.4 V for 40 s. This can be followed by conditioning of electrode in 0.1 M HCl+0.1 M KCl solution by applying a cyclic potential −0.5 to 0.35 V for 25 cycles at 50 mV/s. Gold nanoparticles can be electrodeposited by dipping into a solution of 100 mg/mL of HAuCl₄ and applying a potential of −0.2 V for 30 s. After the electrode preparation, the desired concentration of aptamer can be dropcasted and incubated in a humidity chamber for 16 hours.

Immobilization method 2: For the immobilization on NC, the procedure as described in the first immobilization section can be followed using streptavidin-NC. The immobilization on NC provides higher surface area and a more specific and stronger binding of the aptamer to the surface, but it results in the addition of resistance to the system. Under the same concentrations of aptamer-virion-Ab-GOx, the magnitude of current output can be compared to determine the optimal immobilization strategy.

4. Assembly of the biosensor for SARS-CoV-2 detection. A sandwiched electrochemical detection mechanism can be used consisting of an aptamer targeting the spike glycoprotein, and an antibody (e.g., CR3022/47D11) labeled with glucose oxidase (GOx) specific to the receptor binding domain of the spike glycoprotein of SARS-CoV-2 (see e.g., FIG. 2; see e.g., Song et al. 2020, supra; Wang et al. 2020, supra; Yuan et al. 2020, supra). The aptamer captures the viral antigen, and Ab-GOx binds only if viral antigen is present. GOx is a widely studied enzyme used in glucose biosensors due to its high enzymatic activity and stability; see e.g., Yoo & Sensors (Basel, Switzerland) 10, 4558-4576 (2010); Ferri et al. Journal of diabetes science and technology 5, 1068-1076 (2011); Lee et al. Science Advances 3, (2017). On application of a constant potential, GOx oxidizes glucose, transfers an electron to oxygen, produces hydrogen peroxide, and generates a current output via an electrode that reacts with hydrogen peroxide (see e.g., FIG. 3). The electrode of sensor includes a mediator layer (e.g., Prussian blue) to lower the overvoltage of hydrogen peroxide production process and generate an output current at a lower potential.

The schematics in FIG. 1-3 shows the biotinylated aptamer immobilized on a nitrocellulose membrane coated with streptavidin-NC (i.e., mutant streptavidin with higher affinity to nitrocellulose). The membrane with streptavidin-NC can be prepared by dropcasting streptavidin (1 mg/mL in PBS) on a membrane with 4 mm diameter. The membrane can be dried at 37° C. and stored under dry conditions overnight before use. 10 μL of 20 μM of biotinylated aptamer can be dropcast for 30 min followed by washing in PBS buffer. Following aptamer immobilization, the surface can be blocked by adding 0.1% BSA on the surface, followed by washing.

5 μL of the viral sample can be added and incubated for 15 min. For the control solution to mimic the viral sample, DMEM with 25 mM HEPES, 0.2% BSA, 0.1% FBS and 2 μg/mL of TPCK-Trypsin can be used. The antibody and GOx can be conjugated by using ABCAM'S LIGHTNING-LINK (GOx conjugate kit, #ab102887). The Ab-GOx in PBST can be dropcasted and spread on the membrane surface and incubated for 15 min. This can be followed by washing in PBST. The membrane can be carefully placed on a screen printed DROPSENS 710 electrode, (working and counter electrode: carbon with a Prussian blue layer on top, reference electrode: Ag/AgCl). 50 μL of 100 mM glucose solution (in PBS) can be added on the electrode and chronoamperometric measurement can be performed at −0.2 V using a DROPSENS potentiostat, and the output current can be recorded. The presence of viral antigen generates a current output due to the specific binding of Ab-GOx to the viral molecule, followed by catalytic oxidation of glucose in presence of GOx. The absence of virus, does not cause any Ab-GOx binding to the membrane, generating negligible to no current response.

5. Assessing the biosensor performance and validation. The biosensors undergo a rigorous validation technique to develop a lab based assay for detection of SARS-CoV-2. Using the optimal immobilization strategy, and an aptamer-antibody pair, it is contemplated herein that the biosensors can be fabricated and evaluated for a single use measurement. Biosensors can first be tested against the viral antigen in solution over a clinically relevant range of 10³-10¹¹ viral copies per mL. Sensors can be validated for expected performance in terms of current flux magnitude and velocities, appropriate dependence on redox enzyme, specificity, and dynamic range. Sensors can then be optimized for critical performance metrics including analytical sensitivity and specificity, cross-reactivity, dynamic range, limit of detection, speed and duration of sensing, coefficient of variation of repeated measurements, and operational stability. Sensor interference from compounds present in saliva cam also be tested using artificial saliva samples. Next, the performance of the device can be tested on commercial acquired human saliva samples spiked with known concentrations of viral antigen, constructing a calibration curve. These samples determine the LOD and sensitivity of the sensor using saliva samples. Appropriate statistical methods can be employed to design sample sizes for sufficient power, to determine confidence intervals, and to assess significance. Finally, testing can be performed to assess and calibrate sensor performance over a range of humidity levels, pH, and temperatures.

If the conjugation of the antibody to GOx affects the binding affinity of antibody to its epitope, alternate conjugate strategies can be adopted using chemical linkers for site specific conjugation to GOx. The field of antibody drug conjugates (ADC) has significantly advanced the available technologies such as non-cleavable thioether and peptide linkages for the conjugation of the molecules to antibodies; see e.g., Hasan et al. Current Clinical Pharmacology 13, 236-251 (2018).

Thus, there are several options available for an antibody-GOx conjugate. Systematic evolution of ligands by exponential enrichment (SELEX) can also be performed to identify aptamers targeting the envelope proteins on the viral envelope. If a low current output is recorded due to low concentration of GOx on the sensor surface, the signal can be amplified by using signal processing applications or by integrating electronic amplifiers in the circuit. Data analytical techniques can be used to decrease the effects of noise and variability that can lead to false reads in single tests.

B. Described herein is the repurposing a commercial glucometer to detect SARS-CoV-2 using a test strip. A commercially available OneTouch glucose test strip can be used to fabricate the novel CoV-2 test strip, translated from the experiments described above, for use in a commercial glucometer to sense SARS-CoV-2. Laser cutter and screen printing techniques can be used to fabricate the test strip. The electrode strip undergoes several rounds of optimization steps to meet design requirement using known concentrations of virus in artificial saliva (PICKERING SOLUTIONS, #1700-0313) before testing with human samples. Initially, the test strips can be tested using a lab potentiostat and compared to the commercial available electrodes, before incorporation into a glucometer. The glucometer reads glucose values that reflect the concentration of glucose oxidase enzyme present on the sensor surface. Different GOx concentrations produce different glucose outputs on the glucometer for a fixed glucose concentration. The glucose values displayed on the glucometer (proportional to current output) can be calibrated to the concentration of viral particle present.

The detection device described herein demonstrates that: 1) the fabricated test strip surface captures viral antigen and binds to Ab-GOx to generate a current in the presence of glucose; and 2) glucose readings displayed on the glucometer positively correlate to enzyme concentration on the sensor surface which in turn correlates to number of viral particles present; and, 3) this enzyme amplified detection technology permits detection as a few 1000 viruses in solution, well below the current clinical range of interest.

A point-of-care detection using a commercial glucometer can be used for sensing of viral infection. The sensor output depends on the concentration on antibody-GOx conjugate bound and hence in turn depends on the viral antigen concentration. Knowledge of relationship between glucose values and concentration of viral antigen is used for the diagnosis of viral infection.

1) Fabrication of test strip. The design of the test strip is as follow; see e.g., U.S. Pat. No. 7,462,265, the content of which is incorporated herein by reference in its entirety. As shown in FIG. 5, the sensor strip comprises (bottom up): (A) a base substrate; (B) a conductive layer which includes three electrodes; (C) an insulating layer exposing only part of the electrode where the sample to be tested is dropped; (D) a reagent layer containing mediator for ease of exchange of electrons; (E) an adhesive layer; (F) a hydrophilic nitrocellulose membrane, with a proximal membrane containing aptamer to capture the antigen and freeze dried glucose, and the distal end is the paper sink (13); (G) freeze-dried Ab-GOx; and (H) a top layer. The base substrate can be polyester; an acrylic coating can be applied to improve the ink adhesion. Using a CAD model of electrode mask, the mask is laser cut onto the base substrate. The electrodes can be screen printed using conductive carbon inks from ERCON INC, followed by an insulation layer from ERCON INC (INSULAYER INK). The two working electrodes have a surface area of 0.6 mm² each, and the reference electrode has a surface area of 1.2 mm². The reagent layer is the mediator layer and can consist of a binder, silica, and ferricyanide. This layer is screen printed for two cycles over the working electrodes. The adhesive layer on top can be an acrylic copolymer, the hydrophilic membrane can be a nitrocellulose membrane with streptavidin-NC, with bound biotinylated aptamer to capture the viral antigen. The top layer can be PET, with a small clear portion to see the sample movement on the strip. The overall dimensions can be similar to other test strips to ensure compatibility a glucometer, e.g., LIFESCAN'S reader, or the dimensions can be altered to be compatible with other commercial glucometers.

2) Biosensor evaluation in buffer and artificial saliva samples. Before the addition of freeze dried bio reagents, and aptamer immobilization, GOx is directly dropcast onto the working electrode to compare the functioning of the strips to commercial available glucose strips. This can be done by observing glucose readings displayed on the glucometer at constant glucose concentrations. On observing comparable results, experiments proceed to the immobilization of reagents required for the SARS-CoV-2 testing to complete the fabrication of the CoV strip, as mentioned in the previous section. The single use test-strips can then be validated using viral samples in buffer and in artificial saliva. Around 100 μL of viral particles in solution can be dropped onto the sample chamber, and the solution navigates to the Ab-GOx layer. In presence of the viral particle, Ab-GOx binds, forming a virion-Ab-GOx complex. This complex in turn migrates to the testing zone that contains glucose and aptamer. The aptamer present in the testing zone binds to the complex, bringing the GOx in close proximity to the working electrode and the glucose in solution. The test trip when inserted into the glucometer displays the corresponding glucose values. This process can be repeated for different concentrations of viral samples in buffer to build a calibration plot. The test can also be conducted in artificial saliva samples to test the effect of interferents in saliva. Blind samples with and without viral antigens can be tested to confirm the proper functioning of the test strips.

3) Storage and shelf life of the test strips. It is contemplated herein that the tests above can be followed by more extensive testing and characterization in vitro to establish stability during activity, shelf-life, and tolerance to desiccation and lyophilization. Specifically, a freeze-drying method, with protective agents (e.g., mannitol) and disaccharides (e.g. sucrose, lactose, maltose, and trehalose), can be applied to elongate the shelf-life of biosensor. The protective agents can be added to the nitrocellulose membrane before addition of streptavidin-NC and aptamer. Biosensors can be subjected to a drying protocol different from conventional freeze-drying in that samples can be dried under reduced pressure in a dry atmosphere, but temperatures can be kept above freezing to avoid freeze-injury. Biosensors can be stored in fridge (4° C.), and room temperature (25° C.). Over a period of a month, these sensors' performances can be evaluated in terms of current generation, compared to sensors without protective agents. Also accelerated aging on the strips can be performed to determine test stability over time. Standard accelerated aging protocols can be followed. Accelerated aging allows the estimation of a shelf life based on shorter incubation of a packaged device at higher temperatures. This method is the industry standard, and it provides insight into any problems with materials choice, surface chemistry changes, or assay stability. Tests can be performed at 45° C. for 80 days to estimate the 12-month shelf life.

4) Exploring the scope of use as a POC device. Tests can be performed to optimize the time required for testing the viral samples. An ideal POC device provides results with 2 minutes. Glucose readings can be measured using the glucose reader at different time intervals (e.g., t=1 min, 2 min, 5 min, 10 min) after the addition of saliva samples to investigate the effect of incubation time on the output. The effect of sample volume on the glucose output can also be tested (e.g., 100 μL, 250 μL, 500 μL, 1000 μL, 2000 μL).

Antibody based elements may not always be sufficiently stable overtime. To address this issue, a variety of methods have been developed to stabilize proteins in different matrices. These include sol-gels, hydrogels, and polymeric films. These additives have also been shown to improve specificity, sensitivity, and response time in some applications. A standard array of additives is used (e.g., trehalose, sucralose, etc.) in conjunction with varied drying conditions to identify an optimal composition to maintain the function and robustness of these sensor parts. Storage conditions can be standardized to keep stored chips at constant humidity (via packaging methods) and at a suitable temperature range (as determined by accelerated aging experiments as described above).

C. Described herein is the validation of the SARS-CoV-2 glucometer using human saliva samples and compare the limit of detection and sensitivity to those of RT-PCR. The device can be validated for analytical accuracy against current commercial assays like RT-PCR as well as lateral flow assays on commercially obtained human saliva samples spiked with viral antigen. The human samples spiked with viral copies ranging from 1 to 10¹¹ copies/mL can be tested, and 95% confidence interval can be used to determine limit of detection. Cross-reactivity can be evaluated by testing various microorganism viruses and negative matrixes that might interfere with the functioning of the device.

The point-of-care (POC) detection from saliva samples can allow rapid and facile determination of viral infection in less 2 minutes, in contrast to RT-PCR assays that take around 2 hours and require experienced lab personnel and equipment. Nasal swabs used for RT-PCR assays are a cause of discomfort to the patients. Ease of obtaining the saliva samples is an advantage of this detection technology. RT-PCR and lateral flow assays tests are reagent intensive, and requires expensive swabs whereas the testing methods described herein do not require multiple reagents and swabs as saliva samples are collected in a sterile vial.

It is contemplated herein that the glucometer based biosensor can be superior in terms of analysis time, ease of use, detection limit, as well as at least equivalent in sensitivity compared to current methods like RT-PCR and lateral flow assays for the detection of SARS-CoV-2 viral infection.

1) Evaluation using human saliva samples. The single use disposable point-of-care test strips can be evaluated using human saliva (INNOVATIVE RESEARCH INC.) by spiking in viral particles from concentrations ranging from 1 copy/mL to 10¹¹ copies/mL, with n=5 for each concentration. A calibration curve can be built to determine the relationship between glucometer reading and viral particle concentration. These results can be used to calculate the limit of detection of the device. In parallel, each sample can be validated using an RT-PCR assay to compare the LOD of the device against the gold standard assays. Appropriate statistical methods can be employed to design sample sizes for sufficient power, to determine confidence intervals, and to assess significance. To achieve a 95% confidence interval for a maximum sensitivity and specificity of 95%, a sample set can be created of 73 positive spiked samples and 73 negative spiked samples. These samples can be blinded and tested on 146 different strips.

2) Clinical evaluation. Commercially available human saliva samples can be used with inactivated respiratory related pathogens like influenza strains, MERS-CoV, SARS-CoV, and Adenovirus to determine the cross-reactivity to other viruses; and the device can also be tested against microorganisms like Mycoplasma pneumonia, Streptococcus pyogenes etc., that could interfere with the device. 50 human saliva samples can also be obtained from tested patients with 25 positive and 25 negative to SARS-CoV-2. The samples can be randomized, blind-labeled and tested using the device and gold-standard RT-PCR assays to determine the percentage agreement between the two assays.

The specificity of the sensor depends on the antibody's specificity to its epitope. There are antibodies that are specific to the SARS CoV-2 glycoprotein, and they have nM level binding affinity to the SARS-CoV glycoprotein as well. In some embodiments, two aptamers can be used instead of using an antibody and an aptamer. Aptamers can be selected using Systematic evolution of ligands by exponential enrichment (SELEX), followed by a chemical conjugation with GOx. As the aptamer selection is controlled, aptamers can be designed that bind only to positive targets and aptamer molecules that bind to structurally related molecules can be discarded. Also, aptamers have an added advantage of stability in unique buffer conditions, or in presence of other interferents. Using aptamers also mitigates issues associated with shelf-life of the test strips.

D. Described herein is the determination of sensor performance against emerging coronavirus strains. Sensors can be developed for emerging CoVs or other respiratory viruses that may arise in the coming years. Aptamers and antibodies that target viruses can be used to construct new sensors. SELEX strategies can be used for identification of aptamers, and established assays can be used to identify antibodies for the sensors. The sensor can also be repurposed to detect strains of influenza that affect millions of Americans a year.

Predetermined and appropriate statistical methods can be used to determine significance of results. ANOVA can be used to compare between experimental and control groups with the post-hoc Bonferroni's multiple comparison test performed if significant differences are detected. For validation and calibration curve studies, appropriate statistical methods can be employed to design sample sizes for sufficient power, to determine confidence intervals, and to assess significance. To achieve a 95% confidence interval for a maximum sensitivity and specificity of 95%, a sample set of 73 positive spiked samples and 73 negative spiked samples can be created. The samples can be blinded and run in 146 strips.

For SARS-CoV-2, the minimal target clinical sensitivity can be 90%, and the optimal target sensitivity can be 98%; the minimal target specificity can be 90%, and the optimal target can be >98%. Assuming a sensitivity of 95% versus the reference standard test (RT-PCR) for SARS-CoV-2, 40 confirmed positive saliva samples provide a confidence interval around that sensitivity estimate of +/−6.8%. If both types of saliva samples are considered positives for sensitivity calculations, 80 confirmed positive samples can provide a confidence interval of +/−4.8%. Assuming a specificity of 95%, the same confidence intervals around specificity estimates apply to 40 versus 80 confirmed negative saliva samples. This results in a total of 160 (80 positives) samples total to run on each of the prototype tests. Statistically significant results are defined as a p<0.05. All statistical analyses can be conducted using SPSS 24 (SPSS).

Experiments are performed with 146 purchased samples from single women and men between the ages of 21 and 50, equally distributed between the sexes. Additionally, test samples can be tested from Caucasian, Asian, and African Americans to avoid bias.

The experiments described herein demonstrate: 1) a lab-based assay electrochemical assay for SARS-Cov-2; 2) a portable, facile, point of care SARS-CoV-2 sensor; and, 3) a well-established sensing mechanism implemented for detection of other viral infectious diseases. The test strips can be available to the general public for point of care detection of SARS-CoV-2.

The test strips described herein can be purchased at a drug store or pharmacy, allowing one to test for an infection. Viruses of interest include CoV, SARS-CoV-2, seasonal influenza, highly pathogenic influenza, HIV, Ebola virus, Marburg virus, Lassa virus, respiratory syncytial virus (RSV), human metapneumovirus, as well as pneumonia-associated bacteria, malaria, rickettsial diseases, pneumococcal diseases, etc. Also described herein are test strips that can be used for environmental surveillance for pathogens, including arbo-viral testing in mosquito-trap samples, influenza and West-Nile virus sampling in birds, and zoonotic pathogen testing in bats.

### Example 8: Detection of Target Nucleic Acids

FIG. 15A-15F show alternative designs for the detection device described herein. In one embodiment, the detection device can be used to detect a target nucleic acid. In one embodiment, after an optional nucleic acid extraction from a sample, the target nucleic acid can be contacted with a sequence-specific endonuclease (e.g., a Cas enzyme) and a guide nucleic acid, which is complementary or substantially complementary to at least a portion of the target nucleic acid. In one embodiment, a collateral (non-target) nucleic acid can be added to the solution; upon binding of the sequence-specific endonuclease and guide nucleic acid to the target nucleic acid, the endonuclease cleaves the collateral (non-target) nucleic acid. Cas12a (e.g., ssDNA targets) and Cas13 (e.g., ssRNA targets) are non-limiting examples of sequence-specific endonucleases that demonstrate target-activated collateral cleavage. In one embodiment, a test strip comprises on its surface a detection nucleic acid that is complementary or substantially complementary to at least a portion of the collateral nucleic acid. In one embodiment, cleavage of the collateral nucleic acid permits the collateral nucleic acid to hybridize with the detection nucleic acid on the test strip surface. In one embodiment, the collateral nucleic acid does not hybridize with the detection nucleic until after the collateral nucleic acid is cleaved by the sequence-specific endonuclease and guide nucleic acid, activated by the target nucleic acid (e.g., the endonuclease removes a portion of the collateral nucleic acid that is not complementary to the detection nucleic acid). In some embodiments, glucose oxidase can be introduced to the system and brought into close proximity to the test strip surface either using the collateral nucleic acid or another detection molecule. In some embodiments, glucose reacts with the glucose oxidase, leading to the production of hydrogen peroxide, which can be detected by electrical elements in the detection device as described further herein (see e.g., FIG. 15A-15F).

In one embodiment, the collateral (non-target) nucleic acid comprises a glucose oxidase, e.g., linked to the 3′ or 5′ end of the collateral nucleic acid (see e.g., FIG. 15A). In one embodiment, an aptamer linked to glucose oxidase can be added to the detection system (see e.g., FIG. 15B-15C). In one embodiment, the aptamer binds specifically to at least a portion of the cleaved collateral nucleic acid, e.g., a single-stranded portion of the cleaved collateral nucleic acid that does not hybridize with the detection nucleic acid (see e.g., FIG. 15B). In one embodiment, the aptamer binds specifically to at least a portion of the cleaved collateral nucleic acid that is hybridized to the detection nucleic acid, e.g., a double-stranded portion of the cleaved collateral nucleic acid hybridized with the detection nucleic acid (see e.g., FIG. 15C). In one embodiment, an antibody linked to glucose oxidase can be added to the detection system. In one embodiment, the antibody binds specifically to at least a portion of the cleaved collateral nucleic acid, e.g., a single-stranded or double-stranded portion of the cleaved collateral nucleic acid hybridized with the detection nucleic acid (see e.g., FIG. 15D). In one embodiment, the collateral nucleic acid can be linked to antibody that specifically binds to glucose oxidase; hybridization to the detection nucleic acid of the cleaved nucleic acid linked to the anti-GOx antibody allows for recruitment of glucose oxidase into close proximity to the surface of the test strip (see e.g., FIG. 15E). In one embodiment, the collateral nucleic acid can be linked to one member of an affinity pair (e.g., streptavidin); hybridization to the detection nucleic acid of the cleaved nucleic acid linked to the member of an affinity pair allows for recruitment of glucose oxidase linked to a second member of the affinity pair (e.g., biotin) into close proximity to the surface of the test strip (see e.g., FIG. 15F).

1. A system for detecting at least one target analyte in a biological sample, wherein the system comprises (i) a two binding agent assay, wherein the assay contains a first and second binding agent capable of creating a detectable complex with the at least one target analyte; and (ii) a detection device for detecting the detectable complex, wherein the detection device is an oxidase-based amperometric sensor and the biological sample is present in sweat, saliva, serum, mucus, or blood.

2. The system of claim 1, wherein the amperometric sensor is an oxidase-based, a hydrogenase-based, or dehydrogenase-based amperometric sensor.

3. (canceled)

4. The system of claim 1, wherein the biological sample is sweat, saliva, serum, mucus, or blood.

5. (canceled)

6. The system of claim 1, wherein the detection device is a glucose meter.

7. The system of claim 6, wherein the glucose meter comprises a glucose sensor having a sensor output related to glucose in a biological sample on a test strip.

8. The system of claim 1, wherein the biological sample is mixed with a sugar such as but not limited to glucose.

9. (canceled)

10. The system of claim 1, wherein the first and second binding agents are selected from the group consisting of aptamers, antibodies, proteins, or a combination thereof.

11. The system of claim 1, wherein the first binding agent is an aptamer and the second binding agent is an antibody, wherein the antibody is linked to glucose oxidase.

12. The system of claim 1, wherein the first and second binding agents bind to different sites on the target analyte.

13. The system of claim 12, wherein the first and second binding agents have a Kd for the target analyte from between about 1:1000 to about 1000:1.

14. The system of claim 13, wherein the binding affinity of the first binding agent is weaker than the binding affinity of the second binding agent.

15. The system of claim 1, wherein the target analyte is a whole virus or component thereof.

16. The system of claim 15, wherein the virus is a betacoronavirus, an influenza virus, an HIV virus, or hepatitis virus.

17. The system of claim 16, wherein the target coronavirus analyte is SARS-CoV-2 or a component thereof selected from the group consisting of the spike protein, the membrane protein, the hemagglutinin protein, or the envelope protein.

18. (canceled)

19. The system of claim 1, wherein the target analyte is selected from the group consisting of IgG, IgM, and IgA.

20-33. (canceled)

34. A method for a diagnostic assessment, comprising: (i) collecting a biological sample from a subject, wherein the biological sample is not blood; (ii) adding the biological sample to a test strip in the presence of glucose, wherein the test strip contains a first and second binding agent capable of creating a detectable complex with at least one target analyte, if present in the biological sample; (iii) incubating or not incubating the biological sample with the test strip; (iv) introducing the test strip into a detection device; (v) detecting the level of detectable complex, if any, through a chemical reaction between glucose and glucose oxidase; and (vi) correlating the level of the detectable complex, if produced, with the quantity of the target analyte in the at least one biological sample, if any, thereby providing a diagnostic assessment.

35-48. (canceled)

49. A test strip for use in the system of claim 1, wherein the test strip comprises at least one of the following: (i) a substrate, at least one first and second binding agent and two or more electrodes; (ii) the substrate both first and second binding agents and two or more electrodes; (iii) at least one first and second binding agent and two or more electrodes; or (iv) both first and second binding agents and two or more electrodes.

50-52. (canceled)

53. A kit comprising the test strip of claim 49.

54-75. (canceled)

76. A system for detecting at least one target nucleic acid in a biological sample, wherein the system comprises (i) a sequence-specific endonuclease and guide nucleic acid that cleave a collateral nucleic acid upon specific binding of the target nucleic acid to the endonuclease and guide nucleic acid; (ii) a detection nucleic acid that is capable of creating a detectable complex with the cleaved collateral nucleic acid; and (iii) a detection device for detecting the detectable complex, wherein the detection device is an oxidase-based amperometric sensor and the biological sample is present in sweat, salvia, serum, mucus, or blood.

77-98. (canceled)

99. A method for detecting a target nucleic acid using the system of claim 76, the method comprising:
(i) collecting a biological sample from a subject, and optionally, extracting nucleic acid from the biological sample;
(ii) contacting the biological sample with a sequence-specific endonuclease, guide nucleic acid, and a collateral nucleic acid, wherein such contacting results in cleavage of the collateral nucleic acid, if the target nucleic acid is present;
(iii) adding the biological sample to a test strip in the presence of glucose, wherein the test strip comprises a detection nucleic acid that is capable of creating a detectable complex with the cleaved collateral nucleic acid, if present;
(iv) incubating or not incubating the biological sample with the test strip;
(v) introducing the test strip into a detection device;
(vi) detecting the level of detectable complex, if any, through a chemical reaction between glucose and glucose oxidase; and
(vii) correlating the level of the detectable complex, if produced, with the quantity of the target analyte in the at least one biological sample, thereby providing a diagnostic assessment.

100-109. (canceled)
